SEQUENCE LISTING (36 sequences)

SEQ ID NO: 1 - protein, 450 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 163 aa, synthetic construct
SEQIDNORECOMBINANTHUMANILRHILMAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 133 aa, synthetic construct [sequence omitted]

SEQ ID NO: 6 - protein, 303 aa, synthetic construct
SKNFHLRPRDLISNINVIVLELKGSETTFMCEYADETATIVEFLNRWITFSQSIISTLTGGSSSTKKTQLQLEHLLLDLQMILNGINNYKNPKLTRMLTFKFYMPKKATELKHLQCLEEELKPLEEVLNLAQGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 7 - protein, 452 aa, synthetic construct [sequence omitted]

SEQ ID NO: 8 - protein, 16 aa, synthetic construct
SESSASSDGPHPVITP

SEQ ID NO: 9 - protein, 130 aa, synthetic construct
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 10 - protein, 153 aa, synthetic construct
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 11 - protein, 115 aa, synthetic construct
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 12 - protein, 132 aa, synthetic construct
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 13 - DNA, 2781 bp, synthetic construct
  source 1..2781: mol_type = other DNA
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtggtggctatcgcttccaagctggggggaaagcaggccctggagaccgtccaggcccttctcccagtgctttgccaggctcacggactgacccctgaacaggtggtggcaattgcctcacacgacgggggcaagcaggcactggagactgtccagcggctgctgcctgtcctctgccaggcccacggactcactcctgagcaggtcgtggccattgccagccacgatgggggcaaacaggctctggagaccgtgcagcgcctcctcccagtgctgtgccaggctcatgggctgaccccacagcaggtcgtcgccattgccagtaacggcggggggaagcaggccctcgaaacagtgcagaggctgctgcccgtcttgtgccaagcacacggcctgacacccgagcaggtggtggccatcgcctctcatgacggcggcaagcaggcccttgagacagtgcagagactgttgcccgtgttgtgtcaggcccacgggttgacaccccagcaggtggtcgccatcgccagcaatggcgggggaaagcaggcccttgagaccgtgcagcggttgcttccagtgttgtgccaggcacacggactgacccctcaacaggtggtcgcaatcgccagctacaagggcggaaagcaggctctggagacagtgcagcgcctcctgcccgtgctgtgtcaggctcacggactgacaccacagcaggtggtcgccatcgccagtaacgggggcggcaagcaggctttggagaccgtccagagactcctccccgtcctttgccaggcccacgggttgacacctcagcaggtcgtcgccattgcctccaacaacgggggcaagcaggccctcgaaactgtgcagaggctgctgcctgtgctgtgccaggctcatgggctgacaccccagcaggtggtggccattgcctctaacaacggcggcaaacaggcactggagaccgtgcaaaggctgctgcccgtcctctgccaagcccacgggctcactccacagcaggtcgtggccatcgcctcaaacaatggcgggaagcaggccctggagactgtgcaaaggctgctccctgtgctctgccaggcacacggactgacccctcagcaggtggtggcaatcgcttccaacaacgggggaaagcaggccctcgaaaccgtgcagcgcctcctcccagtgctgtgccaggcacatggcctcacacccgagcaagtggtggctatcgccagccacgacggagggaagcaggctctggagaccgtgcagaggctgctgcctgtcctgtgccaggcccacgggcttactccagagcaggtcgtcgccatcgccagtcatgatggggggaagcaggcccttgagacagtccagcggctgctgccagtcctttgccaggctcacggcttgactcccgagcaggtcgtggccattgcctcaaacattgggggcaaacaggccctggagacagtgcaggccctgctgcccgtgttgtgtcaggcccacggcttgacaccccagcaggtggtcgccattgcctctaatggcggcgggagacccgccttggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 14 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASKLGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASYKGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 15 - DNA, 2778 bp, synthetic construct
  source 1..2778: mol_type = other DNA
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtcgtcgcaatcgccagccatgatggagggaagcaagccctcgaaaccgtgcagcggttgcttcctgtgctctgccaggcccacggccttacccctcagcaggtggtggccatcgcaagtaacggaggaggaaagcaagccttggagacagtgcagcgcctgttgcccgtgctgtgccaggcacacggcctcacaccagagcaggtcgtggccattgcctcccatgacggggggaaacaggctctggagaccgtccagaggctgctgcccgtcctctgtcaagctcacggcctgactccccaacaagtggtcgccatcgcctctaatggcggcgggaagcaggcactggaaacagtgcagagactgctccctgtgctttgccaagctcatgggttgaccccccaacaggtcgtcgctattgcctcaaacggggggggcaagcaggcccttgagactgtgcagaggctgttgccagtgctgtgtcaggctcacgggctcactccacaacaggtggtcgcaattgccagcaacggcggcggaaagcaagctcttgaaaccgtgcaacgcctcctgcccgtgctctgtcaggctcatggcctgacaccacaacaagtcgtggccatcgccagtaataatggcgggaaacaggctcttgagaccgtccagaggctgctcccagtgctctgccaggcacacgggctgacccccgagcaggtggtggctatcgccagcaatattgggggcaagcaggccctggaaacagtccaggccctgctgccagtgctttgccaggctcacgggctcactccccagcaggtcgtggcaatcgcctccaacggcggagggaagcaggctctggagaccgtgcagagactgctgcccgtcttgtgccaggcccacggactcacacctgaacaggtcgtcgccattgcctctcacgatgggggcaaacaagccctggagacagtgcagcggctgttgcctgtgttgtgccaagcccacggcttgactcctcaacaagtggtcgccatcgcctcaaatggcggcggaaaacaagctctggagacagtgcagaggttgctgcccgtcctctgccaagcccacggcctgactccccaacaggtcgtcgccattgccagcaacaacggaggaaagcaggctctcgaaactgtgcagcggctgcttcctgtgctgtgtcaggctcatgggctgacccccgagcaagtggtggctattgcctctaatggaggcaagcaagcccttgagacagtccagaggctgttgccagtgctgtgccaggcccacgggctcacaccccagcaggtggtcgccatcgccagtaacaacgggggcaaacaggcattggaaaccgtccagcgcctgcttccagtgctctgccaggcacacggactgacacccgaacaggtggtggccattgcatcccatgatgggggcaagcaggccctggagaccgtgcagagactcctgccagtgttgtgccaagctcacggcctcacccctcagcaagtcgtggccatcgcctcaaacggggggggccggcctgcactggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 16 - protein, 924 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 17 - DNA, 2814 bp, synthetic construct
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 18 - DNA, 49 bp, synthetic construct
  source 1..49: mol_type = other DNA
ttctccccagccctgctcgtggtgaccgaaggggacaacgccaccttca

SEQ ID NO: 19 - DNA, 2781 bp, synthetic construct
  source 1..2781: mol_type = other DNA
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgaccaagtggttgccatagcgagtaataacggagggaaacaagccctcgaaactgtgcaaaggcttttgcctgtactttgccaggaccacggactcacgcccgaccaggtcgtcgctatagcttcaaacggcgggggcaagcaggctctggaaacagtccaacgccttctgccagttctgtgtcaagaccacgggcttacgccggaccaagtagtggctatcgcctcacacgacggtggtaagcaagcgctggaaaccgtacagcggttgcttccagttctgtgccaagaccacggcttgaccccggaccaagttgtcgcaattgcttcaaatatcggtggtaaacaggccttggagacggtgcaagcccttttgcccgttttgtgtcaagaccacggtttgacgccggaccaggtcgtagccatagcctctcacgacggtggtaagcaagcactggaaaccgtccagcgacttcttccagtactttgtcaagaccatggactgacccccgaccaagtggtggcgattgcatctcacgacggagggaagcaagctctggagactgtccagaggttgctcccggtgctctgccaagaccacggcttgaccccggaccaggtagtggcgattgcaagtaatggcgggggaaagcaagcgctcgagacagttcagcggcttctccccgttttgtgtcaagaccacggccttacgccagaccaagtggtagccatagcatctcacgacggaggtaagcaagcattggaaactgttcagaggctcttgccggtcctgtgtcaggaccacggccttacacctgaccaggtcgttgctattgcaagcaacggcggggggaagcaggcgctggaaacagtgcagcggttgctgcccgtcctttgccaagaccacggcttgactcctgaccaggtggtggctatagcttcacatgacggggggaagcaggctctcgagacagttcagcgccttcttcctgtattgtgccaagaccatgggctcaccccagaccaggttgttgctatagccagtcacgacgggggtaagcaagcacttgaaacggtgcagaggcttctcccagtcctttgtcaggaccatgggctgacacctgaccaggttgttgccatagcgagtaatggcggtggcaagcaggctctggagacggtccaaaggctgttgcccgtcctctgccaagaccacggcttgacacccgaccaagtggtggcgatagcatctcacgacgggggcaagcaagcgttggagactgttcaaaggcttctccctgttctttgccaagaccacggtttgactccagaccaagtcgtagctatagcaagtcacgacggtggtaagcaggctctcgaaactgttcagaggctcctcccagtcctttgtcaagaccatggactgacaccggaccaggtcgtcgctatagcttctaacatcggggggaaacaagctttggaaacagtacaggcccttctgccagttctctgccaagaccacggtcttacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgagaaggggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 20 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 21 - DNA, 2781 bp, synthetic construct
  source 1..2781: mol_type = other DNA
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgaccaagtggttgccatagcgagtcacgacggagggaaacaagccctcgaaactgtgcaaaggcttttgcctgtactttgccaggaccacggactcacgcccgaccaggtcgtcgctatagcttcacatgacgggggcaagcaggctctggaaacagtccaacgccttctgccagttctgtgtcaagaccacgggcttacgccggaccaagtagtggctatcgcctcacacgacggtggtaagcaagcgctggaaaccgtacagcggttgcttccagttctgtgccaagaccacggcttgaccccggaccaagttgtcgcaattgcttcaaatatcggtggtaaacaggccttggagacggtgcaagcccttttgcccgttttgtgtcaagaccacggtttgacgccggaccaggtcgtagccatagcctctaacaatggtggtaagcaagcactggaaaccgtccagcgacttcttccagtactttgtcaagaccatggactgacccccgaccaagtggtggcgattgcatctaatggcggagggaagcaagctctggagactgtccagaggttgctcccggtgctctgccaagaccacggcttgaccccggaccaggtagtggcgattgcaagtaatggcgggggaaagcaagcgctcgagacagttcagcggcttctccccgttttgtgtcaagaccacggccttacgccagaccaagtggtagccatagcatctaacaacggaggtaagcaagcattggaaactgttcagaggctcttgccggtcctgtgtcaggaccacggccttacacctgaccaggtcgttgctattgcaagcaacatcggggggaagcaggcgctggaaacagtgcaggccttgctgcccgtcctttgccaagaccacggcttgactcctgaccaggtggtggctatagcttcacatgacggggggaagcaggctctcgagacagttcagcgccttcttcctgtattgtgccaagaccatgggctcaccccagaccaggttgttgctatagccagtcacgacgggggtaagcaagcacttgaaacggtgcagaggcttctcccagtcctttgtcaggaccatgggctgacacctgaccaggttgttgccatagcgagtaatggcggtggcaagcaggctctggagacggtccaaaggctgttgcccgtcctctgccaagaccacggcttgacacccgaccaagtggtggcgatagcatctaacaacgggggcaagcaagcgttggagactgttcaaaggcttctccctgttctttgccaagaccacggtttgactccagaccaagtcgtagctatagcaagtaataacggtggtaagcaggctctcgaaactgttcagaggctcctcccagtcctttgtcaagaccatggactgacaccggaccaggtcgtcgctatagcttctaacaacggggggaaacaagctttggaaacagtacagcggcttctgccagttctctgccaagaccacggtcttacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgagaaggggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 22 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 23 - DNA, 47 bp, synthetic construct
  source 1..47: mol_type = other DNA
tgtcacctctcctccaccacggcacaagtgacccaggtcaactggga

SEQ ID NO: 24 - DNA, 2781 bp, synthetic construct
  source 1..2781: mol_type = other DNA
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgaccaagtggttgccatagcgagtaatggtggagggaaacaagccctcgaaactgtgcaaaggcttttgcctgtactttgccaggaccacggactcacgcccgaccaggtcgtcgctatagcttcaaacaacgggggcaagcaggctctggaaacagtccaacgccttctgccagttctgtgtcaagaccacgggcttacgccggaccaagtagtggctatcgcctcaaacggcggtggtaagcaagcgctggaaaccgtacagcggttgcttccagttctgtgccaagaccacggcttgaccccggaccaagttgtcgcaattgcttcacacgacggtggtaaacaggccttggagacggtgcaaaggcttttgcccgttttgtgtcaagaccacggtttgacgccggaccaggtcgtagccatagcctctcacgacggtggtaagcaagcactggaaaccgtccagcgacttcttccagtactttgtcaagaccatggactgacccccgaccaagtggtggcgattgcatctaatggcggagggaagcaagctctggagactgtccagaggttgctcccggtgctctgccaagaccacggcttgaccccggaccaggtagtggcgattgcaagtcacgacgggggaaagcaagcgctcgagacagttcagcggcttctccccgttttgtgtcaagaccacggccttacgccagaccaagtggtagccatagcatctcacgacggaggtaagcaagcattggaaactgttcagaggctcttgccggtcctgtgtcaggaccacggccttacacctgaccaggtcgttgctattgcaagccacgacggggggaagcaggcgctggaaacagtgcagcggttgctgcccgtcctttgccaagaccacggcttgactcctgaccaggtggtggctatagcttcaaacggcggggggaagcaggctctcgagacagttcagcgccttcttcctgtattgtgccaagaccatgggctcaccccagaccaggttgttgctatagccagtcacgacgggggtaagcaagcacttgaaacggtgcagaggcttctcccagtcctttgtcaggaccatgggctgacacctgaccaggttgttgccatagcgagtaatggcggtggcaagcaggctctggagacggtccaaaggctgttgcccgtcctctgccaagaccacggcttgacacccgaccaagtggtggcgatagcatctaacatcgggggcaagcaagcgttggagactgttcaagcccttctccctgttctttgccaagaccacggtttgactccagaccaagtcgtagctatagcaagtaataacggtggtaagcaggctctcgaaactgttcagaggctcctcccagtcctttgtcaagaccatggactgacaccggaccaggtcgtcgctatagcttctaacggcggggggaaacaagctttggaaacagtacagcggcttctgccagttctctgccaagaccacggtcttacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgagaaggggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 25 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 26 - DNA, 2781 bp, synthetic construct
  source 1..2781: mol_type = other DNA
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgaccaagtggttgccatagcgagtaataacggagggaaacaagccctcgaaactgtgcaaaggcttttgcctgtactttgccaggaccacggactcacgcccgaccaggtcgtcgctatagcttcaaacaacgggggcaagcaggctctggaaacagtccaacgccttctgccagttctgtgtcaagaccacgggcttacgccggaccaagtagtggctatcgcctcaaacatcggtggtaagcaagcgctggaaaccgtacaggccttgcttccagttctgtgccaagaccacggcttgaccccggaccaagttgtcgcaattgcttcaaatatcggtggtaaacaggccttggagacggtgcaagcccttttgcccgttttgtgtcaagaccacggtttgacgccggaccaggtcgtagccatagcctctaacggcggtggtaagcaagcactggaaaccgtccagcgacttcttccagtactttgtcaagaccatggactgacccccgaccaagtggtggcgattgcatctcacgacggagggaagcaagctctggagactgtccagaggttgctcccggtgctctgccaagaccacggcttgaccccggaccaggtagtggcgattgcaagtaatggcgggggaaagcaagcgctcgagacagttcagcggcttctccccgttttgtgtcaagaccacggccttacgccagaccaagtggtagccatagcatctaacaacggaggtaagcaagcattggaaactgttcagaggctcttgccggtcctgtgtcaggaccacggccttacacctgaccaggtcgttgctattgcaagcaacaacggggggaagcaggcgctggaaacagtgcagcggttgctgcccgtcctttgccaagaccacggcttgactcctgaccaggtggtggctatagcttcaaacatcggggggaagcaggctctcgagacagttcaggcccttcttcctgtattgtgccaagaccatgggctcaccccagaccaggttgttgctatagccagtaacatcgggggtaagcaagcacttgaaacggtgcaggcacttctcccagtcctttgtcaggaccatgggctgacacctgaccaggttgttgccatagcgagtcacgacggtggcaagcaggctctggagacggtccaaaggctgttgcccgtcctctgccaagaccacggcttgacacccgaccaagtggtggcgatagcatctcacgacgggggcaagcaagcgttggagactgttcaaaggcttctccctgttctttgccaagaccacggtttgactccagaccaagtcgtagctatagcaagtaatggcggtggtaagcaggctctcgaaactgttcagaggctcctcccagtcctttgtcaagaccatggactgacaccggaccaggtcgtcgctatagcttctaacaacggggggaaacaagctttggaaacagtacagcggcttctgccagttctctgccaagaccacggtcttacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgagaaggggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 27 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 28 - DNA, 47 bp, synthetic construct
  source 1..47: mol_type = other DNA
ttgtcctccctctagtggctgagcacggtgccaggttccagattcca

SEQ ID NO: 29 - DNA, 93 bp, synthetic construct
  source 1..93: mol_type = other DNA
gctgccttctgcggggcttgccttctggccatgcccttcttctctcccttgcacctgtacctcttggtctttgaataaagcctgagtaggaag

SEQ ID NO: 30 - DNA, 16 bp, synthetic construct
  source 1..16: mol_type = other DNA
agctagcgccgccacc

SEQ ID NO: 31 - RNA, 17 nt, synthetic construct
  source 1..17: mol_type = other RNA
taatacgactcactata

SEQ ID NO: 32 - DNA, 2781 bp, synthetic construct
  source 1..2781: mol_type = other DNA
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 33 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 34 - DNA, 2781 bp, synthetic construct
  source 1..2781: mol_type = other DNA
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 35 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 36 - RNA, 49 nt, synthetic construct
  source 1..49: mol_type = other RNA
tccaggatctcagccttctgcgaagagcaggggtcacttggcagcatca

(19) United States
(12) Patent Application Publication
(10) Pub. No.: US 2026/0234556 A1
(43) Pub. Date: Aug. 13, 2026

(54) PROCESSES FOR GENERATING TIL PRODUCTS USING PD-1/TIGIT TALEN DOUBLE KNOCKDOWN

(71) Applicants: Iovance Biotherapeutics, Inc., San Carlos, CA (US); Cellectis SA, Paris (FR)

(72) Inventors: Rafael CUBAS, Tampa, FL (US); Frederick G. VOGT, West Norriton, PA (US); Hequn YIN, Lithia, FL (US); Marcus MACHIN, Brandon, FL (US); Anand VEERAPATHRAN, Tampa, FL (US); Brittany BUNCH, Tampa, FL (US); Viktoria GONTCHAROVA, Redwood City, CA (US); Rongsu QI, Oviedo, FL (US); Alex BOYNE, Paris (FR); Alexandre JUILLERAT, Paris (FR); Laurent POIROT, Paris (FR)

(21) Appl. No.: 19/108,099

(22) PCT Filed: Sep. 8, 2023
(86) PCT No.: PCT/US2023/073805

§ 371 Date: Feb. 28, 2025

Related U.S. Application Data
(60) Provisional application No. 63/375,194, filed on Sep. 9, 2022.
(60) Provisional application No. 63/376,263, filed on Sep. 19, 2022.
(60) Provisional application No. 63/489,171, filed on Mar. 8, 2023.

(51) Int. Cl. C12N 5/0783; A61K 35/17; A61P 35/00; C12N 9/22; C12N 13/00; C12N 15/87

(52) U.S. Cl. CPC C12N 5/0636; A61K 35/17; A61P 35/00; C12N 9/22; C12N 13/00; C12N 15/87; C12N 2501/2302; C12N 2502/11; C12N 2510/00

(57) ABSTRACT

The present invention provides methods for preparing expanded tumor infiltrating lymphocytes (TILs) having reduced expression of PD-1 and TIGIT using sequential electroporation of two TALEN systems targeting PD-1 and TIGIT. Such TILs find use in therapeutic treatment regimens for cancer patients.

### CROSS-REFERENCE TO RELATED APPLICATIONS

This application is a U.S. National Stage entry of PCT/US2023/073805, filed Sep. 9, 2022, which claims priority to U.S. Provisional Application No. 63/375,194, filed Sep. 9, 2022; U.S. Provisional Application No. 63/376,263, filed Sep. 19, 2022; and U.S. Provisional Application No. 63/489,171, filed Mar. 8, 2023, all of which are herein incorporated by reference in their entireties.

### BACKGROUND OF THE INVENTION

Treatment of bulky, refractory cancers using adoptive autologous transfer of tumor infiltrating lymphocytes (TILs) represents a powerful approach to therapy for patients with poor prognoses. Gattinoni, et al., Nat. Rev. Immunol. 2006, 6, 383-393. TILs are dominated by T cells, and IL-2-based TIL expansion followed by a “rapid expansion process” (REP) has become a preferred method for TIL expansion because of its speed and efficiency. Dudley, et al., Science 2002, 298, 850-54; Dudley, et al., J. Clin. Oncol. 2005, 23, 2346-57; Dudley, et al., J. Clin. Oncol. 2008, 26, 5233-39; Riddell, et al., Science 1992, 257, 238-41; Dudley, et al., J. Immunother. 2003, 26, 332-42. A number of approaches to improve responses to TIL therapy in melanoma and to expand TIL therapy to other tumor types have been explored with limited success, and the field remains challenging. Goff, et al., J. Clin. Oncol. 2016, 34, 2389-97; Dudley, et al., J. Clin. Oncol. 2008, 26, 5233-39; Rosenberg, et al., Clin. Cancer Res. 2011, 17, 4550-57. Combination studies with single immune checkpoint inhibitors have also been described, but further studies are ongoing and additional methods of treatment are needed (Kverneland, et al., Oncotarget, 2020, 11 (22), 2092-2105).

The present invention provides methods for gene-editing at least a portion of the therapeutic population of TILs to enhance their therapeutic effect, by implementing a sequential double KO process that utilizes spaced out delivery of TALEN systems targeting PD-1 and TIGIT to remove risks for chromosomal translocations.

### BRIEF DESCRIPTION OF THE INVENTION

In some embodiments, provided herein is a method for preparing expanded tumor infiltrating lymphocytes (TILs) having reduced expression of PD-1 and TIGIT, comprising:
(a) culturing a first population of TILs in a first cell culture medium comprising IL-2 for about 5-7 days to produce a second population of TILs;
(b) activating the second population of TILs for about 2-4 days, to produce a third population of TILs;
(c) introducing a first TALE nuclease (TALEN) system targeting a first gene selected from the group consisting of a gene encoding PD-1 and a gene encoding TIGIT into at least a portion of the third population of TILs, to produce a fourth population of TILs;
(d) resting the fourth population of TILs in the first cell culture medium comprising IL-2 for about 2 to 3 days;
(e) introducing a second TALEN system targeting a second gene selected from the group consisting of the gene encoding PD-1 and the gene encoding TIGIT into at least a portion of the fourth population of TILs to produce a fifth population of TILs, wherein the first gene and the second gene are different; and
(f) culturing the fifth population of TILs in a second cell culture medium comprising antigen presenting cells (APCs), OKT-3, and IL-2 for about 7-11 days, to produce sixth population of TILs having reduced expression of the first gene and the second gene.

In some embodiments, the step of activating the second population of TILs is performed for about 2 days. In some embodiments, the step of activating the second population of TILs is performed for about 3 days. In some embodiments, the step of activating the second population of TILs is performed for about 4 days. In some embodiments, the step of culturing the first population of TILs is performed for about 5 days. In some embodiments, the step of culturing the first population of TILs is performed for about 6 days. In some embodiments, the step of culturing the first population of TILs is performed for about 7 days. In some embodiments, the step of culturing the fifth population of TILs is performed for about 8 days. In some embodiments, the step of culturing the fifth population of TILs is performed for about 9 days. In some embodiments, the step of culturing the fifth population of TILs is performed for about 10 days. In some embodiments, the step of culturing the fifth population of TILs is performed for about 11 days. In some embodiments, all steps are completed within a period of about 21 days. In some embodiments, all steps are completed within a period of about 19-22 days. In some embodiments, all steps are completed within a period of about 19-21 days. In some embodiments, all steps are completed within a period of about 20-22 days. In some embodiments, all steps are completed within a period of about 24 days. In some embodiments, all steps are completed within a period of about 22 days.

In some embodiments, the method further comprises an overnight resting step after introducing the first and/or the second TALE nuclease system. In some embodiments, the method further comprises an overnight resting step after introducing the first TALE nuclease system and an overnight resting step after introducing the second TALE nuclease system. In some embodiments, the overnight resting step is performed at about 28-32° C. with about 5% CO₂. In some embodiments, step (d) comprises incubating the fourth population of TILs at about 37° C. with about 5% CO₂.

In some embodiments, the step of activating the second population of TILs is performed using anti-CD3 agonist and anti-CD28 agonist. In some embodiments, the step of activating the second population of TILs is performed using TransAct. In some embodiments, the step of activating the second population of TILs is performed using TransAct at 1:17.5 dilution.

In some embodiments, the first TALEN system targets the gene encoding PD-1 and the second TALEN system targets the gene encoding TIGIT. In some embodiments, the first TALEN system targets the gene encoding TIGIT and the second TALEN system targets the gene encoding PD-1. In some embodiments, the target sequence of the PD-1 targeting TALEN system comprise the nucleic acid sequence of SEQ ID NO. 18 and the target sequence of the TIGIT targeting TALEN system comprise the nucleic acid sequence of SEQ ID NO. 23 or 28. In some embodiments, the first TALEN system comprises a first pair of half-TALEs targeting the first gene, wherein the second TALEN system comprises a second pair of half-TALEs targeting the second gene, and wherein the introducing of the first TALEN system comprises a first electroporation of the third population of TILs with a first pair of mRNAs encoding the first pair of half-TALEs and/or the introducing of the second TALEN system comprises a second electroporation of the fifth population of TILs with a second pair of mRNAs encoding the second pair of half-TALEs. In some embodiments, the first pair of half-TALEs comprise the amino acid sequences of SEQ ID NO: 15 and 17. In some embodiments, the second pair of half-TALEs comprise the amino acid sequences of SEQ ID NO: 20 and 22. In some embodiments, the second pair of half-TALEs comprise the amino acid sequences of SEQ ID NO: 25 and 27. In some embodiments, in the first electroporation the first pair of mRNAs is introduced at about 1-2 μg mRNA/million cells of the third population of TILs and/or in the second electroporation the second pair of mRNAs is introduced at about 1-2 μg mRNA/million cells of the fifth population of TILs.

In some embodiments, step (c) is preceded by washing the third population of TILs in a cytoporation buffer. In some embodiments, the first population of TILs is obtained from a tumor tissue resected from a patient. In some embodiments, the first population of TILs is obtained from a sample of tumor tissue produced by surgical resection, needle biopsy, core biopsy, small biopsy, or other means for obtaining tumor tissue from a patient. In some embodiments, the method further comprises digesting in an enzyme media the tumor tissue to produce a tumor digest. In some embodiments, the enzymatic media comprises a DNase. In some embodiments, the enzymatic media comprises a collagenase. In some embodiments, the enzymatic media comprises a neutral protease. In some embodiments, the enzymatic media comprises a hyaluronidase. In some embodiments, the IL-2 concentration is about 10,000 IU/mL to about 5,000 IU/mL or about 1000 IU/ml to 5000 IU/mL.

In some embodiments, one or more of steps (a) to (f) is performed in a closed system. In some embodiments, the transition from step (a) to step (b) occurs without opening the system. In some embodiments, the transition from step (b) to step (c) occurs without opening the system. In some embodiments, the transition from step (c) to step (d) occurs without opening the system. In some embodiments, the transition from step (d) to step (e) occurs without opening the system. In some embodiments, the transition from step (e) to step (f) occurs without opening the system. In some embodiments, the tumor tissue is processed into multiple tumor fragments. In some embodiments, the multiple tumor fragments are added into the closed system. In some embodiments, 150 or fewer of the multiple tumor fragments, 100 or fewer of the multiple tumor fragments, or 50 or fewer of the multiple tumor fragments are added into the closed system.

In some embodiments, a closed system is employed for the TIL expansion, as described herein. In some embodiments, a single bioreactor is employed. In some embodiments, the single bioreactor employed is for example a GREX-10 or a GREX-100M. In some embodiments, the closed system bioreactor is a single bioreactor. In some embodiments, the transition from the priming first expansion to the rapid second expansion involves a scale-up in container size. In some embodiments, the priming first expansion is performed in a smaller container than the rapid second expansion. In some embodiments, the priming first expansion is performed in a GREX-100M and the rapid second expansion is performed in a GREX-500M.

In some embodiments, provided herein is a method for preparing expanded tumor infiltrating lymphocytes (TILs) having reduced expression of PD-1 and TIGIT, comprising:
(a) obtaining a first population of TILs from a tumor sample resected from a patient by processing a tumor sample obtained from the patient into multiple tumor fragments, or from a tumor sample obtained from a patient by surgical resection, needle biopsy, core biopsy, small biopsy, or other means;
(b) culturing the first population of TILs in a first cell culture medium comprising IL-2 for about 5-7 days to produce a second population of TILs;
(c) activating the second population of TILs for about 2-4 days, to produce a third population of TILs;
(d) introducing a first TALE nuclease (TALEN) system targeting a first gene selected from the group consisting of a gene encoding PD-1 and a gene encoding TIGIT into at least a portion of the third population of TILs, to produce a fourth population of TILs;
(e) resting the fourth population of TILs in the first cell culture medium comprising IL-2 for about 2 to 3 days;
(f) introducing a second TALEN system targeting a second gene selected from the group consisting of the gene encoding PD-1 and the gene encoding TIGIT into at least a portion of the fourth population of TILs to produce a fifth population of TILs, wherein the first gene and the second gene are different; and
(g) culturing the fifth population of TILs in a second cell culture medium comprising antigen presenting cells (APCs), OKT-3, and IL-2 for about 7-11 days, to produce sixth population of TILs having reduced expression of the first gene and the second gene.

In some embodiments, the method further comprises: (h) harvesting the sixth population of TILs obtained from step (g). In some embodiments, the method further comprises: (i) transferring the harvested therapeutic TIL population from step (h) to an infusion bag. In some embodiments, the method further comprises: (j) cryopreserving the infusion bag from step (i) using a cryopreservation process.

In some embodiments, the step of activating the second population of TILs is performed for about 2 days. In some embodiments, the step of activating the second population of TILs is performed for about 3 days. In some embodiments, the step of activating the second population of TILs is performed for about 4 days. In some embodiments, the step of culturing the first population of TILs is performed for about 5 days. In some embodiments, the step of culturing the first population of TILs is performed for about 6 days. In some embodiments, the step of culturing the first population of TILs is performed for about 7 days. In some embodiments, the step of culturing the fifth population of TILs is performed for about 8 days. In some embodiments, the step of culturing the fifth population of TILs is performed for about 9 days. In some embodiments, the step of culturing the fifth population of TILs is performed for about 10 days. In some embodiments, the step of culturing the fifth population of TILs is performed for about 11 days. In some embodiments, all steps are completed within a period of about 21 days. In some embodiments, all steps are completed within a period of about 19-22 days. In some embodiments, all steps are completed within a period of about 19-21 days. In some embodiments, all steps are completed within a period of about 20-22 days. In some embodiments, all steps are completed within a period of about 24 days. In some embodiments, all steps are completed within a period of about 22 days.

In some embodiments, the method further comprises an overnight resting step after introducing the first and/or the second TALE nuclease system. In some embodiments, the method further comprises an overnight resting step after introducing the first TALE nuclease system and an overnight resting step after introducing the second TALE nuclease system. In some embodiments, the overnight resting step is performed at about 28-32° C. with about 5% CO₂. In some embodiments, step (d) comprises incubating the fourth population of TILs at about 37° C. with about 5% CO₂.

In some embodiments, the step of activating the second population of TILs is performed using anti-CD3 agonist and anti-CD28 agonist. In some embodiments, the step of activating the second population of TILs is performed using TransAct. In some embodiments, the step of activating the second population of TILs is performed using TransAct at 1:17.5 dilution.

In some embodiments, the first TALEN system targets the gene encoding PD-1 and the second TALEN system targets the gene encoding TIGIT. In some embodiments, the first TALEN system targets the gene encoding TIGIT and the second TALEN system targets the gene encoding PD-1. In some embodiments, the target sequence of the PD-1 targeting TALEN system comprise the nucleic acid sequence of SEQ ID NO. 18 and the target sequence of the TIGIT targeting TALEN system comprise the nucleic acid sequence of SEQ ID NO. 23 or 28. In some embodiments, the first TALEN system comprises a first pair of half-TALEs targeting the first gene, wherein the second TALEN system comprises a second pair of half-TALEs targeting the second gene, and wherein the introducing of the first TALEN system comprises a first electroporation of the third population of TILs with a first pair of mRNAs encoding the first pair of half-TALEs and/or the introducing of the second TALEN system comprises a second electroporation of the fifth population of TILs with a second pair of mRNAs encoding the second pair of half-TALEs. In some embodiments, the first pair of half-TALEs comprise the amino acid sequences of SEQ ID NO: 15 and 17. In some embodiments, the second pair of half-TALEs comprise the amino acid sequences of SEQ ID NO: 20 and 22. In some embodiments, the second pair of half-TALEs comprise the amino acid sequences of SEQ ID NO: 25 and 27. In some embodiments, in the first electroporation the first pair of mRNAs is introduced at about 1-2 μg mRNA/million cells of the third population of TILs and/or in the second electroporation the second pair of mRNAs is introduced at about 1-2 μg mRNA/million cells of the fifth population of TILs.

In some embodiments, step (d) is preceded by washing the third population of TILs in a cytoporation buffer. In some embodiments, the first population of TILs is obtained from a tumor tissue resected from a patient. In some embodiments, the first population of TILs is obtained from a sample of tumor tissue produced by surgical resection, needle biopsy, core biopsy, small biopsy, or other means for obtaining tumor tissue from a patient. In some embodiments, the method further comprises digesting in an enzyme media the tumor tissue to produce a tumor digest. In some embodiments, the enzymatic media comprises a DNase. In some embodiments, the enzymatic media comprises a collagenase. In some embodiments, the enzymatic media comprises a neutral protease. In some embodiments, the enzymatic media comprises a hyaluronidase. In some embodiments, the IL-2 concentration is about 10,000 IU/mL to about 5,000 IU/ml or about 1000 IU/mL to 5000 IU/mL.

In some embodiments, one or more of steps (b) to (g) is performed in a closed system. In some embodiments, the transition from step (b) to step (c) occurs without opening the system. In some embodiments, the transition from step (c) to step (d) occurs without opening the system. In some embodiments, the transition from step (d) to step (e) occurs without opening the system. In some embodiments, the transition from step (e) to step (f) occurs without opening the system. In some embodiments, the transition from step (f) to step (g) occurs without opening the system. In some embodiments, the tumor tissue is processed into multiple tumor fragments. In some embodiments, the multiple tumor fragments are added into the closed system. In some embodiments, 150 or fewer of the multiple tumor fragments, 100 or fewer of the multiple tumor fragments, or 50 or fewer of the multiple tumor fragments are added into the closed system.

In some embodiments, provided herein is a gene-edited population of tumor infiltrating lymphocytes (TILs) comprising an expanded population of TILs having reduced expression of the first gene and the second gene produced by the method disclosed herein.

In some embodiments, about 64% of the expanded population of TILs comprises knockout of both PD-1 and TIGIT. In some embodiments, the expanded population of TILs comprises a therapeutic effective dosage of TILs. In some embodiments, the therapeutically effective dosage of TILs comprises from about 1×10⁹ to about 1×10¹¹ TILs.

In some embodiments, provided herein is a pharmaceutical composition comprising the gene edited population of TILs disclosed herein and a pharmaceutically acceptable carrier.

In some embodiments, provided herein is a method for treating a cancer patient, the method comprising administering a therapeutically effective dose of the gene edited population of TILs or the pharmaceutical composition disclosed herein into the cancer patient. In some embodiments, the cancer is selected from the group consisting of melanoma, metastatic melanoma, ovarian cancer, cervical cancer, non-small-cell lung cancer (NSCLC), metastatic NSCLC, lung cancer, bladder cancer, breast cancer, cancer caused by human papilloma virus, head and neck cancer (including head and neck squamous cell carcinoma (HNSCC)), renal cancer, and renal cell carcinoma.

In some embodiments, provided herein is a method for treating a cancer patient, comprising:
(a) obtaining a first population of TILs from a tumor sample resected from the cancer patient by processing a tumor sample obtained from the cancer patient into multiple tumor fragments, or from a tumor sample obtained from the cancer patient by surgical resection, needle biopsy, core biopsy, small biopsy, or other means;
(b) culturing the first population of TILs in a first cell culture medium comprising IL-2 for about 5-7 days to produce a second population of TILs;
(c) activating the second population of TILs for about 2-4 days, to produce a third population of TILs;
(d) introducing a first TALE nuclease (TALEN) system targeting a first gene selected from the group consisting of a gene encoding PD-1 and a gene encoding TIGIT into at least a portion of the third population of TILs, to produce a fourth population of TILs;
(e) resting the fourth population of TILs in the first cell culture medium comprising IL-2 for about 2 to 3 days;
(f) introducing a second TALEN system targeting a second gene selected from the group consisting of the gene encoding PD-1 and the gene encoding TIGIT into at least a portion of the fourth population of TILs to produce a fifth population of TILs, wherein the first gene and the second gene are different;
(g) culturing the fifth population of TILs in a second cell culture medium comprising antigen presenting cells (APCs), OKT-3, and IL-2 for about 7-11 days, to produce sixth population of TILs having reduced expression of the first gene and the second gene; and
(h) administering a therapeutically effective dosage of the sixth population of TILs to the cancer patient.

In some embodiments, the method further comprises harvesting the sixth population of TILs obtained from step (g). In some embodiments, the method further comprises transferring the harvested therapeutic TIL population to an infusion bag. In some embodiments, the method further comprises cryopreserving the infusion bag using a cryopreservation process. In some embodiments, the cancer is selected from the group consisting of melanoma, metastatic melanoma, ovarian cancer, cervical cancer, non-small-cell lung cancer (NSCLC), metastatic NSCLC, lung cancer, bladder cancer, breast cancer, cancer caused by human papilloma virus, head and neck cancer (including head and neck squamous cell carcinoma (HNSCC)), renal cancer, and renal cell carcinoma.

In some embodiments, the step of activating the second population of TILs is performed for about 2 days. In some embodiments, the step of activating the second population of TILs is performed for about 3 days. In some embodiments, the step of activating the second population of TILs is performed for about 4 days. In some embodiments, the step of culturing the first population of TILs is performed for about 5 days. In some embodiments, the step of culturing the first population of TILs is performed for about 6 days. In some embodiments, the step of culturing the first population of TILs is performed for about 7 days. In some embodiments, the step of culturing the fifth population of TILs is performed for about 8 days. In some embodiments, the step of culturing the fifth population of TILs is performed for about 9 days. In some embodiments, the step of culturing the fifth population of TILs is performed for about 10 days. In some embodiments, the step of culturing the fifth population of TILs is performed for about 11 days. In some embodiments, all steps are completed within a period of about 21 days. In some embodiments, all steps are completed within a period of about 19-22 days. In some embodiments, all steps are completed within a period of about 19-21 days. In some embodiments, all steps are completed within a period of about 20-22 days. In some embodiments, all steps are completed within a period of about 24 days. In some embodiments, all steps are completed within a period of about 22 days.

In some embodiments, the method further comprises an overnight resting step after introducing the first and/or the second TALE nuclease system. In some embodiments, the method further comprises an overnight resting step after introducing the first TALE nuclease system and an overnight resting step after introducing the second TALE nuclease system. In some embodiments, the overnight resting step is performed at about 28-32° C. with about 5% CO₂. In some embodiments, step (d) comprises incubating the fourth population of TILs at about 37° C. with about 5% CO₂.

In some embodiments, the step of activating the second population of TILs is performed using anti-CD3 agonist and anti-CD28 agonist. In some embodiments, the step of activating the second population of TILs is performed using TransAct. In some embodiments, the step of activating the second population of TILs is performed using TransAct at 1:17.5 dilution.

In some embodiments, the first TALEN system targets the gene encoding PD-1 and the second TALEN system targets the gene encoding TIGIT. In some embodiments, the first TALEN system targets the gene encoding TIGIT and the second TALEN system targets the gene encoding PD-1. In some embodiments, the target sequence of the PD-1 targeting TALEN system comprise the nucleic acid sequence of SEQ ID NO. 18 and the target sequence of the TIGIT targeting TALEN system comprise the nucleic acid sequence of SEQ ID NO. 23 or 28. In some embodiments, the first TALEN system comprises a first pair of half-TALEs targeting the first gene, wherein the second TALEN system comprises a second pair of half-TALEs targeting the second gene, and wherein the introducing of the first TALEN system comprises a first electroporation of the third population of TILs with a first pair of mRNAs encoding the first pair of half-TALEs and/or the introducing of the second TALEN system comprises a second electroporation of the fifth population of TILs with a second pair of mRNAs encoding the second pair of half-TALEs. In some embodiments, the first pair of half-TALEs comprise the amino acid sequences of SEQ ID NO: 15 and 17. In some embodiments, the second pair of half-TALEs comprise the amino acid sequences of SEQ ID NO: 20 and 22. In some embodiments, the second pair of half-TALEs comprise the amino acid sequences of SEQ ID NO: 25 and 27. In some embodiments, in the first electroporation the first pair of mRNAs is introduced at about 1-2 μg mRNA/million cells of the third population of TILs and/or in the second electroporation the second pair of mRNAs is introduced at about 1-2 μg mRNA/million cells of the fifth population of TILs.

In some embodiments, step (d) is preceded by washing the third population of TILs in a cytoporation buffer. In some embodiments, the first population of TILs is obtained from a tumor tissue resected from a patient. In some embodiments, the first population of TILs is obtained from a sample of tumor tissue produced by surgical resection, needle biopsy, core biopsy, small biopsy, or other means for obtaining tumor tissue from a patient. In some embodiments, the method further comprises digesting in an enzyme media the tumor tissue to produce a tumor digest. In some embodiments, the enzymatic media comprises a DNase. In some embodiments, the enzymatic media comprises a collagenase. In some embodiments, the enzymatic media comprises a neutral protease. In some embodiments, the enzymatic media comprises a hyaluronidase. In some embodiments, the IL-2 concentration is about 10,000 IU/mL to about 5,000 IU/mL or about 1000 IU/ml to 5000 IU/mL.

In some embodiments, one or more of steps (b) to (g) is performed in a closed system. In some embodiments, the transition from step (b) to step (c) occurs without opening the system. In some embodiments, the transition from step (c) to step (d) occurs without opening the system. In some embodiments, the transition from step (d) to step (e) occurs without opening the system. In some embodiments, the transition from step (e) to step (f) occurs without opening the system. In some embodiments, the transition from step (f) to step (g) occurs without opening the system. In some embodiments, the tumor tissue is processed into multiple tumor fragments. In some embodiments, the multiple tumor fragments are added into the closed system. In some embodiments, 150 or fewer of the multiple tumor fragments, 100 or fewer of the multiple tumor fragments, or 50 or fewer of the multiple tumor fragments are added into the closed system.

In some embodiments, prior to administering a therapeutically effective dosage of the sixth TIL population in step (h), a non-myeloablative lymphodepletion regimen has been administered to the cancer patient. In some embodiments, the method further comprises the step of treating the cancer patient with a high-dose IL-2 regimen starting on the day after administration of the therapeutically effective dosage of the sixth TIL population to the cancer patient in step (h).

In some embodiments, any of the methods as described herein are optionally performed in a closed system.

In some embodiments, provided herein is a method of preparing genetically modified tumor infiltrating lymphocytes (TILs) comprising reduced expression of TIGIT, the method comprising:
(a) introducing into the TILs nucleic acid(s) encoding one or more first Transcription activator-like effector nucleases (TALE-nuclease) able to selectively inactivate by DNA cleavage a gene encoding TIGIT, wherein the one or more first TALE-nucleases comprise a TALE-nuclease that is directed against the nucleic acid sequence of SEQ ID NO: 23 or 28, and optionally introducing one or more second TALE-nucleases able to selectively inactivate by DNA cleavage a gene encoding PD-1; and
(b) expanding the TILs.

In some embodiments, introducing into the TILs nucleic acid(s) encoding the one or more first TALE-nucleases comprises an electroporation step. In some embodiments, the nucleic acid(s) encoding the one or more first TALE-nucleases are RNA and said RNA are introduced into the TILs by electroporation. In some embodiments, the method further comprises prior to the introducing step, a step of activating TILs by culturing the TILs in a cell culture medium in the presence of OKT-3 for about 1-3 days. In some embodiments, the method further comprises after the introducing step and before the expanding step, a step of resting the TILs in a cell culture medium comprising IL-2 for about 1 day. In some embodiments, the method further comprises prior to the introducing step, a step of cryopreserving the TILs followed by thawing and culturing the TILs in a cell culture medium comprising IL-2 for about 1-3 days. In some embodiments, the IL-2 in the resting step is at a concentration of about 3000 IU/ml.

In some embodiments, the one or more first TALE-nucleases are each constituted by a first half-TALE nuclease and a second half-TALE nuclease. In some embodiments, the first half-TALE nuclease is a first fusion protein constituted by a first TALE nucleic acid binding domain fused to a first nuclease catalytic domain and the second half-TALE nuclease is a second fusion protein constituted by a second TALE nucleic acid binding domain fused to a second nuclease catalytic domain. In some embodiments, the first TALE nucleic acid binding domain has a first amino acid sequence and the second TALE nucleic acid binding domain has a second amino acid sequence, and wherein the first amino acid sequence is different from the second amino acid sequence. In some embodiments, the first nuclease catalytic domain has a first amino acid sequence and the second nuclease catalytic domain has a second amino acid sequence, and wherein the first amino acid sequence is the same as the second amino acid sequence. In some embodiments, the first nuclease catalytic domain and the second nuclease catalytic domain both have the amino acid sequence of Fok-I. In some embodiments, the first half-TALE nuclease and the second half-TALE nuclease are capable of forming a heterodimeric DNA cleavage complex to effect DNA cleavage at the target site in the gene encoding TIGIT, and wherein the target site in the gene encoding TIGIT comprises the nucleic acid sequence of SEQ ID NO: 23 or 28. In some embodiments, the first half-TALE nuclease recognizes a first half-target located at a first location in the target site in the gene encoding TIGIT and the second half-TALE nuclease recognizes a second half-target located in a second location in the target site in the gene encoding TIGIT that does not overlap with the first location. In some embodiments, the TALE nuclease comprises an amino acid sequence having at least 70%, 75%, 80%, 85%, 87.5%, 90%, 92.5%, 95%, 97.5%, 98%, or 99% sequence identity with an amino acid sequence selected from the group consisting of SEQ ID NO: 20, SEQ ID NO: 22, SEQ ID NO: 25, and SEQ ID NO: 27. In some embodiments, the TALE-nuclease comprises a sequence selected from the group consisting of SEQ ID NO: 20, SEQ ID NO: 22, SEQ ID NO: 25, and SEQ ID NO: 27. In some embodiments, the first half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 20 and said second half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 22. In some embodiments, the first half-TALE-nuclease comprises the amino acid sequence of SEQ ID NO: 20 and said second half-TALE-nuclease comprises the amino acid sequence of SEQ ID NO: 22. In some embodiments, the first half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 25 and said second half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 27. In some embodiments, the first half-TALE-nuclease comprises the amino acid sequence of SEQ ID NO: 25 and said second half-TALE-nuclease comprises the amino acid sequence of SEQ ID NO: 27.

In some embodiments, the expanded TILs comprise sufficient TILs for administering a therapeutically effective dosage of the TILs to a subject in need thereof. In some embodiments, the therapeutically effective dosage of the expanded TILs comprises from about 1×10⁹ to about 9×10¹⁰ TILs.

In some embodiments, provided herein is a population of expanded tumor infiltrating lymphocytes (TILs) comprising reduced expression of TIGIT and optionally PD-1, the population of expanded TILs being obtainable by the method disclosed herein.

In some embodiments, provided herein is a transcription activator-like effector nuclease (TALE-nuclease) that recognizes and effects DNA cleavage at a target site in a gene encoding TIGIT, wherein the target site comprises the nucleic acid sequence of SEQ ID NO: 23 or 28.

In some embodiments, the TALE-nuclease comprises an amino acid sequence having at least 70%, 75%, 80%, 85%, 87.5%, 90%, 92.5%, 95%, 97.5%, 98%, or 99% sequence identity with an amino acid sequence selected from the group consisting of SEQ ID NO: 20, SEQ ID NO: 22, SEQ ID NO: 25, and SEQ ID NO: 27. In some embodiments, the TALE-nuclease comprises a sequence selected from the group consisting of SEQ ID NO: 20, SEQ ID NO: 22, SEQ ID NO: 25, and SEQ ID NO: 27. In some embodiments, the TALE-nuclease is constituted by a first half-TALE nuclease and a second half-TALE nuclease, and wherein said first half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 20 and said second half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 22. In some embodiments, the first half-TALE-nuclease comprises the amino acid sequence of SEQ ID NO: 20 and said second half-TALE-nuclease comprises the amino acid sequence of SEQ ID NO: 22. In some embodiments, the TALE-nuclease is constituted by a first half-TALE nuclease and a second half-TALE nuclease, and wherein said first half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 25 and said second half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 27. In some embodiments, the first half-TALE-nuclease comprises the amino acid sequence of SEQ ID NO: 25 and said second half-TALE-nuclease comprises the amino acid sequence of SEQ ID NO: 27. In some embodiments, the first half-TALE nuclease is a first fusion protein constituted by a first TALE nucleic acid binding domain fused to a first nuclease catalytic domain and the second half-TALE nuclease is a second fusion protein constituted by a second TALE nucleic acid binding domain fused to a second nuclease catalytic domain. In some embodiments, the first TALE nucleic acid binding domain has a first amino acid sequence and the second TALE nucleic acid binding domain has a second amino acid sequence, and wherein the first amino acid sequence is different from the second amino acid sequence. In some embodiments, the first nuclease catalytic domain has a first amino acid sequence and the second nuclease catalytic domain has a second amino acid sequence, and wherein the first amino acid sequence is the same as the second amino acid sequence. In some embodiments, the first nuclease catalytic domain and the second nuclease catalytic domain both have the amino acid sequence of Fok-I. In some embodiments, the first half-TALE nuclease and the second half-TALE nuclease are capable of forming a heterodimeric DNA cleavage complex to effect DNA cleavage at the target site in the gene encoding TIGIT. In some embodiments, the first half-TALE nuclease recognizes a first half-target located at a first location in the target site in the gene encoding TIGIT and the second half-TALE nuclease recognizes a second half-target located in a second location in the target site in the gene encoding TIGIT that does not overlap with the first location.

In some embodiments, the TALE-nuclease is encoded by a nucleic acid sequence having at least 90%, 92.5%, 95%, 97.5%, 98%, or 99% sequence identity with a nucleic acid sequence selected from the group consisting of SEQ ID NO: 19, SEQ ID NO: 21, SEQ ID NO: 24, and SEQ ID NO: 26. In some embodiments, the TALE-nuclease is encoded by a nucleic acid sequence selected from the group consisting of SEQ ID NO: 19, SEQ ID NO: 21, SEQ ID NO: 24, and SEQ ID NO: 26. In some embodiments, the TALE-nuclease is constituted by a first half-TALE nuclease and a second half-TALE nuclease, and wherein said first half-TALE-nuclease is encoded by a nucleic acid sequence having at least 90%, 92.5%, 95%, 97.5%, 98%, or 99% sequence identity with SEQ ID NO: 19 and said second half-TALE-nuclease is encoded by a nucleic acid sequence having at least 90%, 92.5%, 95%, 97.5%, 98%, or 99% sequence identity with SEQ ID NO: 21. In some embodiments, the first half-TALE-nuclease is encoded by the nucleic acid sequence of SEQ ID NO: 20 and said second half-TALE-nuclease is encoded by the nucleic acid sequence of SEQ ID NO: 22. In some embodiments, the TALE-nuclease is constituted by a first half-TALE nuclease and a second half-TALE nuclease, and wherein said first half-TALE-nuclease is encoded by a nucleic acid sequence having at least 90%, 92.5%, 95%, 97.5%, 98%, or 99% sequence identity with SEQ ID NO: 24 and said second half-TALE-nuclease is encoded by a nucleic acid sequence having at least 90%, 92.5%, 95%, 97.5%, 98%, or 99% sequence identity with SEQ ID NO: 26. In some embodiments, the first half-TALE-nuclease is encoded by the amino acid sequence of SEQ ID NO: 24 and said second half-TALE-nuclease is encoded by the amino acid sequence of SEQ ID NO: 26.

In some embodiments, provided herein is a method for expanding genetically modified tumor infiltrating lymphocytes (TILs) into a therapeutic population of TILs comprising reduced expression of TIGIT, the method comprising:
(a) obtaining and/or receiving a first population of TILs from a tumor sample;
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) introducing into the TILs nucleic acid(s) encoding one or more first Transcription activator-like effector nucleases (TALE-nuclease) able to selectively inactivate by DNA cleavage a gene encoding TIGIT, wherein the one or more first TALE-nucleases comprise a TALE-nuclease that is directed against a target site in the gene encoding TIGIT, wherein the target site comprises the nucleic acid sequence of SEQ ID NO: 23 or 28, and wherein the transition from step (c) to step (d) occurs without opening the system;
(e) performing a second expansion by culturing the TILs obtained from step (d) in a cell culture medium comprising IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (d) to step (e) occurs without opening the system; and
(f) harvesting the therapeutic population of TILs obtained from step (e), wherein the transition from step (e) to step (f) occurs without opening the system.

In some embodiments, the nucleic acid(s) encoding the one or more first TALE-nucleases are RNA. In some embodiments, introducing the nucleic acid(s) encoding the one or more first TALE-nucleases are introduced into the TILs by electroporation. In some embodiments, the method further comprises prior to the introducing step, a step of activating TILs by culturing the TILs in a cell culture medium in the presence of OKT-3 for about 1-3 days. In some embodiments, OKT-3 is at a concentration of about 300 ng/ml. In some embodiments, the method further comprises after the introducing step and before the second expansion step, a step of resting the TILs in a cell culture medium comprising IL-2 for about 1 day. In some embodiments, the IL-2 in the resting step is at a concentration of about 3000 IU/ml. In some embodiments, steps (a) through (f) are performed in about 13 days to about 29 days, optionally about 15 days, about 16 days, about 17 days, about 18 days, about 19 days, about 20 days, about 21 days, about 22 days, about 23 days, about 24 days, or about 25 days. In some embodiments, the nucleic acid(s) encoding the one or more first TALE-nucleases are RNA, and said RNA are introduced into the TILs by electroporation.

In some embodiments, the one or more first TALE-nucleases are each constituted by a first half-TALE nuclease and a second half-TALE nuclease. In some embodiments, the first half-TALE nuclease is a first fusion protein constituted by a first TALE nucleic acid binding domain fused to a first nuclease catalytic domain and the second half-TALE nuclease is a second fusion protein constituted by a second TALE nucleic acid binding domain fused to a second nuclease catalytic domain. In some embodiments, the first TALE nucleic acid binding domain has a first amino acid sequence and the second TALE nucleic acid binding domain has a second amino acid sequence, and wherein the first amino acid sequence is different from the second amino acid sequence. In some embodiments, the first nuclease catalytic domain has a first amino acid sequence and the second nuclease catalytic domain has a second amino acid sequence, and wherein the first amino acid sequence is the same as the second amino acid sequence. In some embodiments, the first nuclease catalytic domain and the second nuclease catalytic domain both have the amino acid sequence of Fok-I. In some embodiments, the first half-TALE nuclease and the second half-TALE nuclease are capable of forming a heterodimeric DNA cleavage complex to effect DNA cleavage at the target site. In some embodiments, the first half-TALE nuclease recognizes a first half-target located at a first location in the target site and the second half-TALE nuclease recognizes a second half-target located in a second location in the target site that does not overlap with the first location. In some embodiments, the TALE-nuclease comprises an amino acid sequence having at least 70%, 75%, 80%, 85%, 87.5%, 90%, 92.5%, 95%, 97.5%, 98%, or 99% sequence identity with an amino acid sequence selected from the group consisting of SEQ ID NO: 20, SEQ ID NO: 22, SEQ ID NO: 25, and SEQ ID NO: 27. In some embodiments, the TALE-nuclease comprises a sequence selected from the group consisting of SEQ ID NO: 20, SEQ ID NO: 22, SEQ ID NO: 25, and SEQ ID NO: 27. In some embodiments, the first half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 20 and said second half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 22. In some embodiments, the first half-TALE-nuclease comprises the amino acid sequence of SEQ ID NO: 20 and said second half-TALE-nuclease comprises the amino acid sequence of SEQ ID NO: 22. In some embodiments, the first half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 25 and said second half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 27. In some embodiments, the first half-TALE-nuclease comprises the amino acid sequence of SEQ ID NO: 25 and said second half-TALE-nuclease comprises the amino acid sequence of SEQ ID NO: 27.

In some embodiments, the TILs harvested comprises sufficient TILs for administering a therapeutically effective dosage of the TILs to a subject in need thereof. In some embodiments, the therapeutically effective dosage of the TILs comprises from about 1×10⁹ to about 9×10¹⁰ TILs.

### BRIEF DESCRIPTION OF THE DRAWINGS

FIG. 1: Viability of TILs after sequential electroporation.

FIG. 2: LAG3 and PD-1 KO efficiency in CD3+ (FIG. 2A), CD8+ (FIG. 2B) and CD4+ (FIG. 2C) TILs.

FIG. 3: Fold expansion (FIG. 3A) and viability (FIG. 3B) of concomitantly and sequentially electroporated TILs after REP.

FIG. 4: Cell growth after stimulation at different days (FIG. 4A), 1^{st} electroporation PD-1 KO efficiency (FIG. 4B), and 2^{nd} electroporation PD-1 KO efficiency (FIG. 4C).

FIG. 5: Percentage of TIL growth over 3-day rest period with stimulation on different days (Day 0, 3, 5, 7).

FIG. 6: PD-1 and TIGIT KO efficiencies on total CD3+ TILs with 4-day and 2-day stimulation.

FIG. 7: PD-1 and TIGIT KO efficiencies on total CD8+ TILs with 4-day and 2-day stimulation.

FIG. 8: PD-1 and TIGIT KO efficiencies on total CD4+ TILs with 4-day and 2-day stimulation.

FIG. 9: Frequency of PD-1 and TIGIT expression on CD3+ TILs.

FIG. 10: Shows an exemplary processes for expanding TILs by sequential electroporation of TALE-nucleases directed against a target sequence in PD-1 and TIGIT.

FIG. 11: Shows cell recovery after electroporation of different concentrations of PD-1 TALEN mRNA.

FIG. 12: Shows cell viability after electroporation of different concentrations of PD-1 TALEN mRNA.

FIG. 13: Shows cell doubling after electroporation of different concentrations of PD-1 TALEN mRNA.

FIG. 14: Shows extrapolated total viable cells after electroporation of different concentrations of PD-1 TALEN mRNA.

FIG. 15: Shows interim PD-1 KO efficiency after electroporation of different concentrations of PD-1 TALEN mRNA.

FIG. 16: Shows final PD-1 KO efficiency after electroporation of different concentrations of PD-1 TALEN mRNA.

FIG. 17: Shows cell recovery after electroporation of different concentrations of TIGIT TALEN mRNA.

FIG. 18: Shows cell viability after electroporation of different concentrations of TIGIT TALEN mRNA.

FIG. 19: Shows cell doubling after electroporation of different concentrations of TIGIT TALEN mRNA.

FIG. 20: Shows extrapolated total viable cells after electroporation of different concentrations of TIGIT TALEN mRNA.

FIG. 21: Shows interim TIGIT KO efficiency after electroporation of different concentrations of TIGIT TALEN mRNA.

FIG. 22: Shows final TIGIT KO efficiency after electroporation of different concentrations of TIGIT TALEN mRNA.

FIGS. 23A and 23B: Shows an exemplary process flow for genetic modification of PD-1 and TIGIT as part of a preferred embodiment of a TIL expansion method, including (A) an alternative electroporation method and (B) a method for scaling up TIL cultures during a rapid expansion.

FIGS. 24A and 24B: Show adoptive transfer of PD1/TIGIT dKO TIL leads to increased tumor control compared to PD1 sKO and mock control.

FIG. 25: Shows similar recovery of TIL 21 days post adoptive transfer between PD1 sKO and PD1/TIGIT dKO cells.

FIGS. 26A-26C: show that strongest KO efficiency observed for 39233/39234 TALEN pair, but overall strong KO efficiency observed for both TALEN mRNA pairs at concentrations of 2-4 ug/million cells.

FIGS. 27A-27B: show PD-1 and TIGIT KO efficiency using flow cytometry and ddPCR assays.

FIGS. 28A-28D: Show PD-1 and TIGIT KO efficiency measured by flow cytometry or ddPCR.

FIG. 29: Shows IL-2 independent proliferation assay results for PD1/TIGIT dKO TILs showed no proliferation.

FIGS. 30A and 30B: show the single and double KO efficiencies for PD1 and LAG3, respectively.

FIGS. 31A and 31B: show fold expansion and viability observed for LAG3 single and double KO TILs.

FIGS. 32A-32F: show decreased CD69, CD39, CD127, Eomes, Tbet and TOX expression in single and double KO TILs.

FIGS. 33A-33D: show similar levels of IFNγ and TNFα expression and killing activity were observed in single and double KO TILs.

FIGS. 34A-34C: show LAG3 and PD1 KO efficiency, fold expansion during REP and viability after REP, respectively.

FIGS. 35A-35C: show PD-1, TIGIT and LAG3 KO efficiency, respectively.

FIG. 36: shows PD-1 on-target hyperbola fit options.

FIGS. 37A-37F: show PD-1 off-target signals for Candidates 3, 1, 19, 9, 17, and 4, respectively.

FIG. 38: shows TIGIT on-target hyperbola fit options.

FIGS. 39A-39E: show TIGIT off-target signals for Candidates 1, 2, 10, 12, and 17, respectively.

### DESCRIPTION OF THE INVENTION

### I. Definitions

Unless defined otherwise, all technical and scientific terms used herein have the same meaning as is commonly understood by one of skill in the art to which this invention belongs. All patents and publications referred to herein are incorporated by reference in their entireties.

The terms “co-administration,” “co-administering,” “administered in combination with,” “administering in combination with,” “simultaneous,” and “concurrent,” as used herein, encompass administration of two or more active pharmaceutical ingredients (in a preferred embodiment of the present invention, for example, a plurality of TILs) to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “ex vivo” refers to an event which involves treating or performing a procedure on a cell, tissue and/or organ which has been removed from a subject's body. Aptly, the cell, tissue and/or organ may be returned to the subject's body in a method of surgery or treatment.

The term “rapid expansion” means an increase in the number of antigen-specific TILs of at least about 3-fold (or 4-, 5-, 6-, 7-, 8-, or 9-fold) over a period of a week, more preferably at least about 10-fold (or 20-, 30-, 40-, 50-, 60-, 70-, 80-, or 90-fold) over a period of a week, or most preferably at least about 100-fold over a period of a week. A number of rapid expansion protocols are described herein.

By “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and M1 macrophages. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein, including, but not limited to bulk TILs and expanded TILs (“REP TILs” or “post-REP TILs”). TIL cell populations can include genetically modified TILs.

By “population of cells” (including TILs) herein is meant a number of cells that share common traits. In general, populations generally range from 1×10⁶ to 1×10¹⁰ in number, with different TIL populations comprising different numbers. For example, initial growth of primary TILs in the presence of IL-2 results in a population of bulk TILs of roughly 1×10⁸ cells. REP expansion is generally done to provide populations of 1.0×10⁹ to 1.0×10¹¹ cells for infusion.

By “cryopreserved TILs” herein is meant that TILs, either primary, bulk, or expanded (REP TILs), are treated and stored in the range of about −150° C. to −60° C. General methods for cryopreservation are also described elsewhere herein, including in the Examples. For clarity, “cryopreserved TILs” are distinguishable from frozen tissue samples which may be used as a source of primary TILs.

By “thawed cryopreserved TILs” herein is meant a population of TILs that was previously cryopreserved and then treated to return to room temperature or higher, including but not limited to cell culture temperatures or temperatures wherein TILs may be administered to a patient.

TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally and alternatively, TILs can be functionally defined by their ability to infiltrate solid tumors upon reintroduction into a patient.

The term “cryopreservation media” or “cryopreservation medium” refers to any medium that can be used for cryopreservation of cells. Such media can include media comprising 7% to 10% DMSO. Exemplary media include CryoStor CS10, Hyperthermasol, as well as combinations thereof. The term “CS10” refers to a cryopreservation medium which is obtained from Stemcell Technologies or from Biolife Solutions. The CS10 medium may be referred to by the trade name “CryoStor® CS10”. The CS10 medium is a serum-free, animal component-free medium which comprises DMSO. In some embodiments, the CS10 medium comprises 10% DMSO.

The term “central memory T cell” refers to a subset of T cells that in the human are CD45R0+ and constitutively express CCR7 (CCR7^{hi}) and CD62L (CD62^{hi}). The surface phenotype of central memory T cells also includes TCR, CD3, CD127 (IL-7R), and IL-15R. Transcription factors for central memory T cells include BCL-6, BCL-6B, MBD2, and BMI1. Central memory T cells primarily secret IL-2 and CD40L as effector molecules after TCR triggering. Central memory T cells are predominant in the CD4 compartment in blood, and in the human are proportionally enriched in lymph nodes and tonsils.

The term “effector memory T cell” refers to a subset of human or mammalian T cells that, like central memory T cells, are CD45R0+, but have lost the constitutive expression of CCR7 (CCR7^{lo}) and are heterogeneous or low for CD62L expression (CD62L^{lo}). The surface phenotype of central memory T cells also includes TCR, CD3, CD127 (IL-7R), and IL-15R. Transcription factors for central memory T cells include BLIMP1. Effector memory T cells rapidly secret high levels of inflammatory cytokines following antigenic stimulation, including interferon-γ, IL-4, and IL-5. Effector memory T cells are predominant in the CD8 compartment in blood, and in the human are proportionally enriched in the lung, liver, and gut. CD8+ effector memory T cells carry large amounts of perforin.

The term “closed system” refers to a system that is closed to the outside environment. Any closed system appropriate for cell culture methods can be employed with the methods of the present invention. Closed systems include, for example, but are not limited to, closed G-containers. Once a tumor segment is added to the closed system, the system is no opened to the outside environment until the TILs are ready to be administered to the patient.

The terms “fragmenting,” “fragment,” and “fragmented,” as used herein to describe processes for disrupting a tumor, includes mechanical fragmentation methods such as crushing, slicing, dividing, and morcellating tumor tissue as well as any other method for disrupting the physical structure of tumor tissue.

The terms “peripheral blood mononuclear cells” and “PBMCs” refers to a peripheral blood cell having a round nucleus, including lymphocytes (T cells, B cells, NK cells) and monocytes. When used as an antigen presenting cell (PBMCs are a type of antigen-presenting cell), the peripheral blood mononuclear cells are preferably irradiated allogeneic peripheral blood mononuclear cells.

The terms “peripheral blood lymphocytes” and “PBLs” refer to T cells expanded from peripheral blood. In some embodiments, PBLs are separated from whole blood or apheresis product from a donor. In some embodiments, PBLs are separated from whole blood or apheresis product from a donor by positive or negative selection of a T cell phenotype, such as the T cell phenotype of CD3+ CD45+.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UHCT1 clone, also known as T3 and CD3E. Other anti-CD3 antibodies include, for example, otelixizumab, teplizumab, and visilizumab.

The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/ml, MACS GMP CD3 pure, Miltenyi Biotech, Inc., San Diego, CA, USA) and muromonab or variants, conservative amino acid substitutions, glycoforms, or biosimilars thereof. The amino acid sequences of the heavy and light chains of muromonab are given in Table 1 (SEQ ID NO:1 and SEQ ID NO:2). A hybridoma capable of producing OKT-3 is deposited with the American Type Culture Collection and assigned the ATCC accession number CRL 8001. A hybridoma capable of producing OKT-3 is also deposited with European Collection of Authenticated Cell Cultures (ECACC) and assigned Catalogue No. 86022706.

TABLE 1
Amino acid sequences of muromonab.

| Identifier | Sequence (One-Letter Amino Acid Symbols) | |
|---|---|---|
| SEQ ID NO: 1 | QVQLQQSGAE LARPGASVKM SCKASGYTFT RYTMHWVKQR PGQGLEWIGY INPSRGYTNY | 60 |
| Muromonab heavy | NQKFKDKATL TTDKSSSTAY MQLSSLTSED SAVYYCARYY DDHYCLDYWG QGTTLTVSSA | 120 |
| chain | KTTAPSVYPL APVCGGTTGS SVTLGCLVKG YFPEPVTLTW NSGSLSSGVH TFPAVLQSDL | 180 |
| | YTLSSSVTVT SSTWPSQSIT CNVAHPASST KVDKKIEPRP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 2 | QIVLTQSPAI MSASPGEKVT MTCSASSSVS YMNWYQQKSG TSPKRWIYDT SKLASGVPAH | 60 |
| Muromonab light | FRGSGSGTSY SLTISGMEAE DAATYYCQQW SSNPFTFGSG TKLEINRADT APTVSIFPPS | 120 |
| chain | SEQLTSGGAS VVCFLNNFYP KDINVKWKID GSERQNGVLN SWTDQDSKDS TYSMSSTLTL | 180 |
| | TKDEYERHNS YTCEATHKTS TSPIVKSFNR NEC | 213 |

The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. The amino acid sequence of recombinant human IL-2 suitable for use in the invention is given in Table 2 (SEQ ID NO:3). For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, NH, USA (CELLGRO GMP) or ProSpec-Tany TechnoGene Ltd., East Brunswick, NJ, USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The amino acid sequence of aldesleukin suitable for use in the invention is given in Table 2 (SEQ ID NO:4). The term IL-2 also encompasses pegylated forms of IL-2, as described herein, including the pegylated IL2 prodrug bempegaldesleukin (NKTR-214, pegylated human recombinant IL-2 as in SEQ ID NO:4 in which an average of 6 lysine residues are N⁶ substituted with [(2,7-bis{[methylpoly(oxyethylene)]carbamoyl}-9H-fluoren-9-yl) methoxy]carbonyl), which is available from Nektar Therapeutics, South San Francisco, CA, USA, or which may be prepared by methods known in the art, such as the methods described in Example 19 of International Patent Application Publication No. WO 2018/132496 A1 or the method described in Example 1 of U.S. Patent Application Publication No. US 2019/0275133 A1, the disclosures of which are incorporated by reference herein. Bempegaldesleukin (NKTR-214) and other pegylated IL-2 molecules suitable for use in the invention are described in U.S. Patent Application Publication No. US 2014/0328791 A1 and International Patent Application Publication No. WO 2012/065086 A1, the disclosures of which are incorporated by reference herein. Alternative forms of conjugated IL-2 suitable for use in the invention are described in U.S. Pat. Nos. 4,766,106, 5,206,344, 5,089,261 and 4,902,502, the disclosures of which are incorporated by reference herein. Formulations of IL-2 suitable for use in the invention are described in U.S. Pat. No. 6,706,289, the disclosure of which is incorporated by reference herein.

In some embodiments, an IL-2 form suitable for use in the present invention is THOR-707, available from Synthorx, Inc. The preparation and properties of THOR-707 and additional alternative forms of IL-2 suitable for use in the invention are described in U.S. Patent Application Publication Nos. US 2020/0181220 A1 and US 2020/0330601 A1, the disclosures of which are incorporated by reference herein. In some embodiments, and IL-2 form suitable for use in the invention is an interleukin 2 (IL-2) conjugate comprising: an isolated and purified IL-2 polypeptide; and a conjugating moiety that binds to the isolated and purified IL-2 polypeptide at an amino acid position selected from K35, T37, R38, T41, F42, K43, F44, Y45, E61, E62, E68, K64, P65, V69, L72, and Y107, wherein the numbering of the amino acid residues corresponds to SEQ ID NO:5. In some embodiments, the amino acid position is selected from T37, R38, T41, F42, F44, Y45, E61, E62, E68, K64, P65, V69, L72, and Y107. In some embodiments, the amino acid position is selected from T37, R38, T41, F42, F44, Y45, E61, E62, E68, P65, V69, L72, and Y107. In some embodiments, the amino acid position is selected from T37, T41, F42, F44, Y45, P65, V69, L72, and Y107. In some embodiments, the amino acid position is selected from R38 and K64. In some embodiments, the amino acid position is selected from E61, E62, and E68. In some embodiments, the amino acid position is at E62. In some embodiments, the amino acid residue selected from K35, T37, R38, T41, F42, K43, F44, Y45, E61, E62, E68, K64, P65, V69, L72, and Y107 is further mutated to lysine, cysteine, or histidine. In some embodiments, the amino acid residue is mutated to cysteine. In some embodiments, the amino acid residue is mutated to lysine. In some embodiments, the amino acid residue selected from K35, T37, R38, T41, F42, K43, F44, Y45, E61, E62, E68, K64, P65, V69, L72, and Y107 is further mutated to an unnatural amino acid. In some embodiments, the unnatural amino acid comprises N6-azidoethoxy-L-lysine (AzK), N6-propargylethoxy-L-lysine (Prak), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl) alanine, 2-amino-3-((2-((3-(benzyloxy)-3-oxopropyl)amino)ethyl) selanyl) propanoic acid, 2-amino-3-(phenylselanyl) propanoic, or selenocysteine. In some embodiments, the IL-2 conjugate has a decreased affinity to IL-2 receptor α (IL-2Rα) subunit relative to a wild-type IL-2 polypeptide. In some embodiments, the decreased affinity is about 10%, 20%, 30%, 40%, 50%, 60%, 70%, 80%, 90%, 95%, 99%, or greater than 99% decrease in binding affinity to IL-2Ra relative to a wild-type IL-2 polypeptide. In some embodiments, the decreased affinity is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 500-fold, 1000-fold, or more relative to a wild-type IL-2 polypeptide. In some embodiments, the conjugating moiety impairs or blocks the binding of IL-2 with IL-2Rα. In some embodiments, the conjugating moiety comprises a water-soluble polymer. In some embodiments, the additional conjugating moiety comprises a water-soluble polymer. In some embodiments, each of the water-soluble polymers independently comprises polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof. In some embodiments, each of the water-soluble polymers independently comprises PEG. In some embodiments, the PEG is a linear PEG or a branched PEG. In some embodiments, each of the water-soluble polymers independently comprises a polysaccharide. In some embodiments, the polysaccharide comprises dextran, polysialic acid (PSA), hyaluronic acid (HA), amylose, heparin, heparan sulfate (HS), dextrin, or hydroxyethyl-starch (HES). In some embodiments, each of the water-soluble polymers independently comprises a glycan. In some embodiments, each of the water-soluble polymers independently comprises polyamine. In some embodiments, the conjugating moiety comprises a protein. In some embodiments, the additional conjugating moiety comprises a protein. In some embodiments, each of the proteins independently comprises an albumin, a transferrin, or a transthyretin. In some embodiments, each of the proteins independently comprises an Fc portion. In some embodiments, each of the proteins independently comprises an Fc portion of IgG. In some embodiments, the conjugating moiety comprises a polypeptide. In some embodiments, the additional conjugating moiety comprises a polypeptide. In some embodiments, each of the polypeptides independently comprises a XTEN peptide, a glycine-rich homoamino acid polymer (HAP), a PAS polypeptide, an elastin-like polypeptide (ELP), a CTP peptide, or a gelatin-like protein (GLK) polymer. In some embodiments, the isolated and purified IL-2 polypeptide is modified by glutamylation. In some embodiments, the conjugating moiety is directly bound to the isolated and purified IL-2 polypeptide. In some embodiments, the conjugating moiety is indirectly bound to the isolated and purified IL-2 polypeptide through a linker. In some embodiments, the linker comprises a homobifunctional linker. In some embodiments, the homobifunctional linker comprises Lomant's reagent dithiobis(succinimidylpropionate) DSP, 3′ 3′-dithiobis(sulfosuccinimidyl proprionate) (DTSSP), disuccinimidyl suberate (DSS), bis(sulfosuccinimidyl) suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-(3′-(2′-pyridyldithio) propionamido) butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide). In some embodiments, the linker comprises a heterobifunctional linker. In some embodiments, the heterobifunctional linker comprises N-succinimidyl 3-(2-pyridyldithio) propionate (sPDP), long-chain N-succinimidyl 3-(2-pyridyldithio) propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio) toluene (sMPT), sulfosuccinimidyl-6-[α-methyl-α-(2-pyridyldithio) toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl) butyrate (sMPB), sulfosuccinimidyl-4-(p-maleimidophenyl) butyrate (sulfo-sMPB), N-(γ-maleimidobutyryloxy) succinimide ester (GMBs), N-(γ-maleimidobutyryloxy) sulfosuccinimide ester (sulfo-GMBs), succinimidyl 6-((iodoacetyl)amino) hexanoate (sIAX), succinimidyl 6-[6-(((iodoacetyl)amino) hexanoyl)amino]hexanoate (sIAXX), succinimidyl 4-(((iodoacetyl)amino)methyl)cyclohexane-1-carboxylate (sIAC), succinimidyl 6-(((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl) butyric acid hydrazide (MPBH), 4-(N-maleimidomethyl)cyclohexane-1-carboxyl-hydrazide-8 (M2C2H), 3-(2-pyridyldithio) propionyl hydrazide (PDPH), N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido) hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenyl amino) hexanoate (sANPAH), sulfosuccinimidyl-6-(4′-azido-2′-nitrophenylamino) hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4 (4-azidophenyl) 1,3′-dithiopropionate (sADP), N-sulfosuccinimidyl (4-azidophenyl)-1,3′-dithiopropionate (sulfo-sADP), sulfosuccinimidyl 4-(p-azidophenyl) butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), 1-(p-azidosalicylamido)-4-(iodoacetamido) butane (AsIB), N-[4-(p-azidosalicylamido)butyl]-3′-(2′-pyridyldithio) propionamide (APDP), benzophenone-4-iodoacetamide, p-azidobenzoyl hydrazide (ABH), 4-(p-azidosalicylamido)butylamine (AsBA), or p-azidophenyl glyoxal (APG). In some embodiments, the linker comprises a cleavable linker, optionally comprising a dipeptide linker. In some embodiments, the dipeptide linker comprises Val-Cit, Phe-Lys, Val-Ala, or Val-Lys. In some embodiments, the linker comprises a non-cleavable linker. In some embodiments, the linker comprises a maleimide group, optionally comprising maleimidocaproyl (mc), succinimidyl-4-(N-maleimidomethyl)cyclohexane-1-carboxylate (sMCC), or sulfosuccinimidyl-4-(N-maleimidomethyl)cyclohexane-1-carboxylate (sulfo-sMCC). In some embodiments, the linker further comprises a spacer. In some embodiments, the spacer comprises p-aminobenzyl alcohol (PAB), p-aminobenzyoxycarbonyl (PABC), a derivative, or an analog thereof. In some embodiments, the conjugating moiety is capable of extending the serum half-life of the IL-2 conjugate. In some embodiments, the additional conjugating moiety is capable of extending the serum half-life of the IL-2 conjugate. In some embodiments, the IL-2 form suitable for use in the invention is a fragment of any of the IL-2 forms described herein. In some embodiments, the IL-2 form suitable for use in the invention is pegylated as disclosed in U.S. Patent Application Publication No. US 2020/0181220 A1 and U.S. Patent Application Publication No. US 2020/0330601 A1. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 80% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 polypeptide comprises an N-terminal deletion of one residue relative to SEQ ID NO:5. In some embodiments, the IL-2 form suitable for use in the invention lacks IL-2R alpha chain engagement but retains normal binding to the intermediate affinity IL-2R beta-gamma signaling complex. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 90% sequence identity to SEQ ID NO: 5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 95% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 98% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5.

In some embodiments, an IL-2 form suitable for use in the invention is nemvaleukin alfa, also known as ALKS-4230 (SEQ ID NO:6), which is available from Alkermes, Inc. Nemvaleukin alfa is also known as human interleukin 2 fragment (1-59), variant (Cys¹²⁵>Ser⁵¹), fused via peptidyl linker (⁶⁰GG⁶¹) to human interleukin 2 fragment (62-132), fused via peptidyl linker (¹³³GSGGGS¹³⁸) to human interleukin 2 receptor α-chain fragment (139-303), produced in Chinese hamster ovary (CHO) cells, glycosylated; human interleukin 2 (IL-2) (75-133)-peptide [Cys¹²⁵ (51)>Ser]-mutant (1-59), fused via a G₂ peptide linker (60-61) to human interleukin 2 (IL-2) (4-74)-peptide (62-132) and via a GSG3S peptide linker (133-138) to human interleukin 2 receptor α-chain (IL2R subunit alpha, IL2Rα, IL2RA) (1-165)-peptide (139-303), produced in Chinese hamster ovary (CHO) cells, glycoform alfa. The amino acid sequence of nemvaleukin alfa is given in SEQ ID NO:6. In some embodiments, nemvaleukin alfa exhibits the following post-translational modifications: disulfide bridges at positions: 31-116, 141-285, 184-242, 269-301, 166-197 or 166-199, 168-199 or 168-197 (using the numbering in SEQ ID NO:6), and glycosylation sites at positions: N187, N206, T212 using the numbering in SEQ ID NO:6. The preparation and properties of nemvaleukin alfa, as well as additional alternative forms of IL-2 suitable for use in the invention, is described in U.S. Patent Application Publication No. US 2021/0038684 A1 and U.S. Pat. No. 10,183,979, the disclosures of which are incorporated by reference herein. In some embodiments, an IL-2 form suitable for use in the invention is a protein having at least 80%, at least 90%, at least 95%, or at least 90% sequence identity to SEQ ID NO:6. In some embodiments, an IL-2 form suitable for use in the invention has the amino acid sequence given in SEQ ID NO:6 or conservative amino acid substitutions thereof. In some embodiments, an IL-2 form suitable for use in the invention is a fusion protein comprising amino acids 24-452 of SEQ ID NO:7, or variants, fragments, or derivatives thereof. In some embodiments, an IL-2 form suitable for use in the invention is a fusion protein comprising an amino acid sequence having at least 80%, at least 90%, at least 95%, or at least 90% sequence identity to amino acids 24-452 of SEQ ID NO:7, or variants, fragments, or derivatives thereof. Other IL-2 forms suitable for use in the present invention are described in U.S. Pat. No. 10,183,979, the disclosures of which are incorporated by reference herein. Optionally, in some embodiments, an IL-2 form suitable for use in the invention is a fusion protein comprising a first fusion partner that is linked to a second fusion partner by a mucin domain polypeptide linker, wherein the first fusion partner is IL-1Ra or a protein having at least 98% amino acid sequence identity to IL-1Ra and having the receptor antagonist activity of IL-Ra, and wherein the second fusion partner comprises all or a portion of an immunoglobulin comprising an Fc region, wherein the mucin domain polypeptide linker comprises SEQ ID NO:8 or an amino acid sequence having at least 90% sequence identity to SEQ ID NO:8 and wherein the half-life of the fusion protein is improved as compared to a fusion of the first fusion partner to the second fusion partner in the absence of the mucin domain polypeptide linker.

TABLE 2
Amino acid sequences of interleukins.

| Identifier | Sequence (One-Letter Amino Acid Symbols) | |
|---|---|---|
| SEQ ID NO: 3 | MAPTSSSTKK TQLQLEHLLL DLQMILNGIN NYKNPKLTRM LTFKFYMPKK ATELKHLQCL | 60 |
| recombinant | EEELKPLEEV LNLAQSKNFH LRPRDLISNI NVIVLELKGS ETTFMCEYAD ETATIVEFLN | 120 |
| human IL-2 | RWITFCQSII STLT | 134 |
| (rhIL-2) | | |
| SEQ ID NO: 4 | PTSSSTKKTQ LQLEHLLLDL QMILNGINNY KNPKLTRMLT FKFYMPKKAT ELKHLQCLEE | 60 |
| Aldesleukin | ELKPLEEVLN LAQSKNFHLR PRDLISNINV IVLELKGSET TFMCEYADET ATIVEFLNRW | 120 |
| | ITFSQSIIST LT | 132 |
| SEQ ID NO: 5 | APTSSSTKKT QLQLEHLLLD LQMILNGINN YKNPKLTRML TFKFYMPKKA TELKHLQCLE | 60 |
| IL-2 form | EELKPLEEVL NLAQSKNFHL RPRDLISNIN VIVLELKGSE TTFMCEYADE TATIVEFLNR | 120 |
| | WITFCQSIIS TLT | 133 |
| SEQ ID NO: 6 | SKNFHLRPRD LISNINVIVL ELKGSETTEM CEYADETATI VEFLNRWITF SQSIISTLTG | 60 |
| Nemvaleukin | GSSSTKKTQL QLEHLLLDLQ MILNGINNYK NPKLTRMLTF KFYMPKKATE LKHLQCLEEE | 120 |
| alfa | LKPLEEVLNL AQGSGGGSEL CDDDPPEIPH ATFKAMAYKE GTMLNCECKR GFRRIKSGSL | 180 |
| | YMLCTGNSSH SSWDNQCQCT SSATRNTTKQ VTPQPEEQKE RKTTEMQSPM QPVDQASLPG | 240 |
| | HCREPPPWEN EATERIYHFV VGQMVYYQCV QGYRALHRGP AESVCKMTHG KTRWTQPQLI | 300 |
| | CTG | 303 |
| SEQ ID NO: 7 | MDAMKRGLCC VLLLCGAVEV SARRPSGRKS SKMQAFRIWD VNQKTFYLRN NQLVAGYLQG | 60 |
| IL-2 form | PNVNLEEKID VVPIEPHALF LGIHGGKMCL SCVKSGDETR LQLEAVNITD LSENRKQDKR | 120 |
| | FAFIRSDSGP TTSFESAACP GWFLCTAMEA DQPVSLTNMP DEGVMVTKFY FQEDESGSGG | 180 |
| | ASSESSASSD GPHPVITESR ASSESSASSD GPHPVITESR EPKSSDKTHT CPPCPAPELL | 240 |
| | GGPSVFLFPP KPKDTLMISR TPEVTCVVVD VSHEDPEVKF NWYVDGVEVH NAKTKPREEQ | 300 |
| | YNSTYRVVSV LTVLHQDWLN GKEYKCKVSN KALPAPIEKT ISKAKGQPRE PQVYTLPPSR | 360 |
| | EEMTKNQVSL TCLVKGFYPS DIAVEWESNG QPENNYKTTP PVLDSDGSFF LYSKLTVDKS | 420 |
| | RWQQGNVFSC SVMHEALHNH YTQKSLSLSP GK | 452 |
| SEQ ID NO: 8 | SESSASSDGP HPVITP | 16 |
| mucin domain | | |
| polypeptide | | |
| SEQ ID NO: 9 | MHKCDITLQE IIKTLNSLTE QKTLCTELTV TDIFAASKNT TEKETFCRAA TVLRQFYSHH | 60 |
| recombinant | EKDTRCLGAT AQQFHRHKQL IRFLKRLDRN LWGLAGLNSC PVKEANQSTL ENFLERLKTI | 120 |
| human IL-4 | MREKYSKCSS | 130 |
| (rhIL-4) | | |
| SEQ ID NO: 10 | MDCDIEGKDG KQYESVLMVS IDQLLDSMKE IGSNCLNNEF NFFKRHICDA NKEGMFLFRA | 60 |
| recombinant | ARKLRQFLKM NSTGDFDLHL LKVSEGTTIL LNCTGQVKGR KPAALGEAQP TKSLEENKSL | 120 |
| human IL-7 | KEQKKLNDLC FLKRLLQEIK TCWNKILMGT KEH | 153 |
| (rhIL-7) | | |
| SEQ ID NO: 11 | MNWVNVISDL KKIEDLIQSM HIDATLYTES DVHPSCKVTA MKCFLLELQV ISLESGDASI | 60 |
| recombinant | HDTVENLIIL ANNSLSSNGN VTESGCKECE ELEEKNIKEF LQSFVHIVQM FINTS | 115 |
| human IL-15 | | |
| (rhIL-15) | | |
| SEQ ID NO: 12 | MQDRHMIRMR QLIDIVDQLK NYVNDLVPEF LPAPEDVETN CEWSAFSCFQ KAQLKSANTG | 60 |
| recombinant | NNERIINVSI KKLKRKPPST NAGRRQKHRL TCPSCDSYEK KPPKEFLERE KSLLQKMIHQ | 120 |
| human IL-21 | HLSSRTHGSE DS | 132 |
| (rhIL-21) | | |

When “an anti-tumor effective amount”, “a tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the tumor infiltrating lymphocytes (e.g. secondary TILs or genetically modified cytotoxic lymphocytes) described herein may be administered at a dosage of 10⁴ to 10¹¹ cells/kg body weight (e.g., 10⁵ to 10⁶, 10⁵ to 10¹⁰, 10⁵ to 10¹¹, 10⁶ to 10¹⁰, 10⁶ to 10¹¹, 10⁷ to 10¹¹, 10⁷ to 10¹⁰, 10⁸ to 10¹¹, 10⁸ to 10¹⁰, 10⁹ to 10¹¹, or 10⁹ to 10¹⁰ cells/kg body weight), including all integer values within those ranges. TILs (including in some cases, genetically modified cytotoxic lymphocytes) compositions may also be administered multiple times at these dosages. The TILs (including, in some cases, genetically engineered TILs) can be administered by using infusion techniques that are commonly known in immunotherapy (see, e.g., Rosenberg, et al., New Eng. J. of Med. 1988, 319, 1676). The optimal dosage and treatment regime for a particular patient can readily be determined by one skilled in the art of medicine by monitoring the patient for signs of disease and adjusting the treatment accordingly.

The term “hematological malignancy”, “hematologic malignancy” or terms of correlative meaning refer to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, acute lymphoblastic leukemia (ALL), chronic lymphocytic lymphoma (CLL), small lymphocytic lymphoma (SLL), acute myelogenous leukemia (AML), chronic myelogenous leukemia (CML), multiple myeloma, acute monocytic leukemia (AMoL), Hodgkin's lymphoma, and non-Hodgkin's lymphomas. The term “B cell hematological malignancy” refers to hematological malignancies that affect B cells.

The term “liquid tumor” refers to an abnormal mass of cells that is fluid in nature. Liquid tumor cancers include, but are not limited to, leukemias, myelomas, and lymphomas, as well as other hematological malignancies. TILs obtained from liquid tumors may also be referred to herein as marrow infiltrating lymphocytes (MILs). TILs obtained from liquid tumors, including liquid tumors circulating in peripheral blood, may also be referred to herein as PBLs. The terms MIL, TIL, and PBL are used interchangeably herein and differ only based on the tissue type from which the cells are derived.

The term “microenvironment,” as used herein, may refer to the solid or hematological tumor microenvironment as a whole or to an individual subset of cells within the microenvironment. The tumor microenvironment, as used herein, refers to a complex mixture of “cells, soluble factors, signaling molecules, extracellular matrices, and mechanical cues that promote neoplastic transformation, support tumor growth and invasion, protect the tumor from host immunity, foster therapeutic resistance, and provide niches for dominant metastases to thrive,” as described in Swartz, et al., Cancer Res., 2012, 72, 2473. Although tumors express antigens that should be recognized by T cells, tumor clearance by the immune system is rare because of immune suppression by the microenvironment.

In some embodiments, the invention includes a method of treating a cancer with a population of TILs, wherein a patient is pre-treated with non-myeloablative chemotherapy prior to an infusion of TILs according to the invention. In some embodiments, the population of TILs may be provided wherein a patient is pre-treated with nonmyeloablative chemotherapy prior to an infusion of TILs according to the present invention. In some embodiments, the non-myeloablative chemotherapy is cyclophosphamide 60 mg/kg/d for 2 days (days 27 and 26 prior to TIL infusion) and fludarabine 25 mg/m2/d for 5 days (days 27 to 23 prior to TIL infusion). In some embodiments, after non-myeloablative chemotherapy and TIL infusion (at day 0) according to the invention, the patient receives an intravenous infusion of IL-2 intravenously at 720,000 IU/kg every 8 hours to physiologic tolerance.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) on the patient prior to the introduction of the TILs of the invention.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

The terms “non-myeloablative chemotherapy,” “non-myeloablative lymphodepletion,” “NMALD,” “NMA LD,” “NMA-LD,” and any variants of the foregoing, are used interchangeably to indicate a chemotherapeutic regimen designed to deplete the patient's lymphoid immune cells while avoiding depletion of the patient's myeloid immune cells. Typically, the patient receives a course of non-myeloablative chemotherapy prior to the administration of tumor infiltrating lymphocytes to the patient as described herein.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, California) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody. The term variant also includes pegylated antibodies or proteins.

The term “deoxyribonucleotide” encompasses natural and synthetic, unmodified and modified deoxyribonucleotides. Modifications include changes to the sugar moiety, to the base moiety and/or to the linkages between deoxyribonucleotide in the oligonucleotide.

The term “RNA” defines a molecule comprising at least one ribonucleotide residue. The term “ribonucleotide” defines a nucleotide with a hydroxyl group at the 2′ position of a b-D-ribofuranose moiety. The term RNA includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Nucleotides of the RNA molecules described herein may also comprise non-standard nucleotides, such as non-naturally occurring nucleotides or chemically synthesized nucleotides or deoxynucleotides. These altered RNAs can be referred to as analogs or analogs of naturally-occurring RNA.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in therapeutic compositions of the invention is contemplated. Additional active pharmaceutical ingredients, such as other drugs, can also be incorporated into the described compositions and methods.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (Clq) of the classical complement system.

The term “antigen” refers to a substance that induces an immune response. In some embodiments, an antigen is a molecule capable of being bound by an antibody or a TCR if presented by major histocompatibility complex (MHC) molecules. The term “antigen”, as used herein, also encompasses T cell epitopes. An antigen is additionally capable of being recognized by the immune system. In some embodiments, an antigen is capable of inducing a humoral immune response or a cellular immune response leading to the activation of B lymphocytes and/or T lymphocytes. In some cases, this may require that the antigen contains or is linked to a Th cell epitope. An antigen can also have one or more epitopes (e.g., B- and T-epitopes). In some embodiments, an antigen will preferably react, typically in a highly specific and selective manner, with its corresponding antibody or TCR and not with the multitude of other antibodies or TCRs which may be induced by other antigens.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to certain receptors can be made using knowledge and skill in the art of injecting test subjects with suitable antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

### II. Methods for Preparing Expanded TILs Having Reduced Expression of PD-1 and TIGIT Using Sequential Electroporation of Two TALEN Systems

Embodiments of the present invention are directed to methods for preparing expanded tumor infiltrating lymphocytes (TILs) having reduced expression of PD-1 and TIGIT using sequential electroporation of two TALEN systems targeting PD-1 and TIGIT.

### A. Overview: TIL Expansion+ TALEN Gene-Editing

Embodiments disclosed herein provide a method for expanding TILs into a therapeutic population that further comprises gene-editing at least a portion of the TILs by a TALE method to produce TILs having reduced expression of PD-1 and TIGIT. According to particular embodiments, the use of a TALE method during the TIL expansion process causes expression of PD-1 and TIGIT to be silenced or reduced in at least a portion of the therapeutic population of TILs.

As used herein, “gene-editing,” “gene editing,” and “genome editing” refer to a type of genetic modification in which DNA is permanently modified in the genome of a cell, e.g., DNA is inserted, deleted, modified or replaced within the cell's genome. In some embodiments, gene-editing causes the expression of a DNA sequence to be silenced (sometimes referred to as a gene knockout) or inhibited/reduced (sometimes referred to as a gene knockdown). In accordance with embodiments of the present invention, gene-editing technology is used to enhance the effectiveness of a therapeutic population of TILs.

A method for expanded TILs having reduced expression of PD-1 and TIGIT may be carried out in accordance with any embodiment of the methods described herein or by modifying the methods described in WO 2012/129201 A1, WO 2018/081473 A1, WO 2018/129332 A1, or WO 2018/182817 A1, the contents of which are herein incorporated by reference in their entireties, to incorporate steps for reducing the expression of PD-1 and TIGIT in TILs as described herein. Briefly, in some embodiments, the method for expanding TILs comprises a first expansion step of culturing a population of TILs in a first cell culture medium comprising IL-2 for about 7-14 days (the “pre-REP” step), an activation step, a step of introducing a first TALEN system targeting a first gene selected from the group consisting of PD-1 and TIGIT, a resting step, a step of introducing a second TALEN system targeting a second gene selected from the group consisting of PD-1 and TIGIT, wherein the second gene and the first gene are not the same, and a second expansion step of culturing a population of TILs after the second introducing step in a second cell culture medium comprising antigen presenting cells (APCs), OKT-3, and IL-2 for about 7-14 days (the “REP” step).

Examples of systems, methods, and compositions for altering the expression of one or more target gene sequences by a TALE method, and which may be used in accordance with embodiments of the present invention, are described in U.S. Pat. No. 8,586,526 and WO 2018/007263 A1, the contents of which are incorporated by reference herein in their entireties.

TALE stands for “Transcription Activator-Like Effector” proteins, which include TALENs (“Transcription Activator-Like Effector Nucleases”). A method of using a TALE system for gene-editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Life Technologies (Grand Island, NY, USA). TALE and TALEN methods suitable for use in the present invention are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein in their entireties.

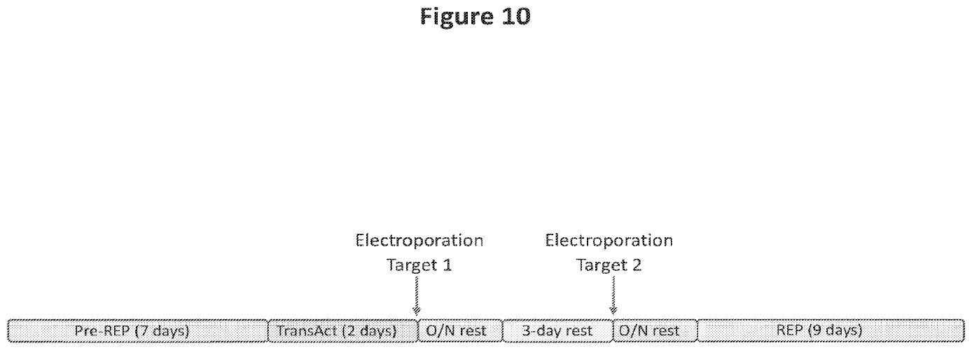

An exemplary process for production and expansion of TILs having reduced expression of PD-1 and TIGIT is depicted in FIG. 10, wherein the expanded TILs have been genetically modified via TALEN gene editing by introducing sequentially into the TILs nucleic acids, such as mRNAs, encoding TALEN systems targeting PD-1 and TIGIT.

In some embodiments, the method comprises:
(a) culturing a first population of TILs in a first cell culture medium comprising IL-2 for about 5-7 days to produce a second population of TILs;
(b) activating the second population of TILs for 2-4 days, to produce a third population of TILs;
(c) introducing a first TALE nuclease (TALEN) system targeting a first gene selected from the group consisting of PD-1 and TIGIT into at least a portion of the third population of TILs, to produce a fourth population of TILs;
(d) resting the fourth population of TILs in the first cell culture medium comprising IL-2 for 3 days;
(e) introducing a second TALEN system targeting a second gene selected from the group consisting of PD-1 and TIGIT into at least a portion of the fourth population of TILs to produce a fifth population of TILs, wherein the first and second gene are different; and
(f) culturing the fifth population of TILs in a second cell culture medium comprising antigen presenting cells (APCs), OKT-3, and IL-2 for about 7-11 days, to produce sixth population of TILs having reduced expression of PD-1 and TIGIT.

In some embodiments, the method comprises a first expansion step (step (a)), an activation step (step (b)), two TALEN-mediated gene-editing steps (steps (c) and (e)) separated by a resting period (step (d)), followed by a second expansion step (step (f)).

In alternative embodiments, the first expansion step and the activation step may be combined in part or in full. In some embodiments, the activation step may be considered a continuation of the first expansion step. For example, the activation step is performed in the first cell culture medium comprising IL-2, by adding an anti-CD3 agonist and anti-CD28 agonist, such as TransAct.

### B. Obtain Patient Tumor Sample

In general, TILs are initially obtained from a patient tumor sample (“primary TILs”) and then expanded into a larger population for further manipulation as described herein, wherein the expanded TILs have been genetically modified via TALEN gene editing by introducing sequentially into the TILs nucleic acids, such as mRNAs, encoding TALEN systems targeting PD-1 and TIGIT.

A patient tumor sample may be obtained using methods known in the art, generally via surgical resection, needle biopsy, core biopsy, small biopsy, or other means for obtaining a sample that contains a mixture of tumor and TIL cells. In some embodiments, multilesional sampling is used. In some embodiments, surgical resection, needle biopsy, core biopsy, small biopsy, or other means for obtaining a sample that contains a mixture of tumor and TIL cells includes multilesional sampling (i.e., obtaining samples from one or more tumor cites and/or locations in the patient, as well as one or more tumors in the same location or in close proximity). In general, the tumor sample may be from any solid tumor, including primary tumors, invasive tumors or metastatic tumors. The tumor sample may also be a liquid tumor, such as a tumor obtained from a hematological malignancy. The solid tumor may be of skin tissue. In some embodiments, useful TILs are obtained from a melanoma.

Once obtained, the tumor sample is generally fragmented using sharp dissection into small pieces of between 1 to about 8 mm³, with from about 2-3 mm³ being particularly useful. In some embodiments, the TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 mcg/mL gentamicine, 30 units/ml of DNase and 1.0 mg/ml of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding TILs or methods treating a cancer.

As indicated above, in some embodiments, the TILs are derived from solid tumors. In some embodiments, the solid tumors are not fragmented. In some embodiments, the solid tumors are not fragmented and are subjected to enzymatic digestion as whole tumors. In some embodiments, the tumors are digested in in an enzyme mixture comprising collagenase, DNase, and neutral protease. In some embodiments, the tumors are digested in in an enzyme mixture comprising collagenase, DNase, and neutral protease for 1-2 hours. In some embodiments, the tumors are digested in in an enzyme mixture comprising collagenase, DNase, and neutral protease for 1-2 hours at 37° C., 5% CO₂. In some embodiments, the tumors are digested in in an enzyme mixture comprising collagenase, DNase, and neutral protease for 1-2 hours at 37° C., 5% CO₂ with rotation. In some embodiments, the tumors are digested overnight with constant rotation. In some embodiments, the tumors are digested overnight at 37° C., 5% CO₂ with constant rotation. In some embodiments, the whole tumor is combined with the enzymes to form a tumor digest reaction mixture.

In some embodiments, the tumor is reconstituted with the lyophilized enzymes in a sterile buffer. In some embodiments, the buffer is sterile HBSS.

In some embodiments, the enzyme mixture comprises collagenase. In some embodiments, the collagenase is collagenase IV. In some embodiments, the working stock for the collagenase is a 100 mg/ml 10× working stock.

In some embodiments, the enzyme mixture comprises DNAse. In some embodiments, the working stock for the DNAse is a 10,000 IU/ml 10× working stock.

In some embodiments, the enzyme mixture comprises hyaluronidase. In some embodiments, the working stock for the hyaluronidase is a 10-mg/ml 10× working stock.

In some embodiments, the enzyme mixture comprises 10 mg/ml collagenase, 1000 IU/ml DNAse, and 1 mg/ml hyaluronidase.

In some embodiments, the enzyme mixture comprises 10 mg/ml collagenase, 500 IU/ml DNAse, and 1 mg/ml hyaluronidase.

In some embodiments, the enzyme mixture comprises neutral protease. In some embodiments, the working stock for the neutral protease is reconstituted at a concentration of 175 DMC U/mL.

In some embodiments, the enzyme mixture comprises neutral protease, DNase, and collagenase.

In some embodiments, the enzyme mixture comprises 10 mg/ml collagenase, 1000 IU/ml DNase, and 0.31 DMC U/ml neutral protease. In some embodiments, the enzyme mixture comprises 10 mg/ml collagenase, 500 IU/ml DNase, and 0.31 DMC U/ml neutral protease.

In general, the harvested cell suspension is called a “primary cell population” or a “freshly harvested” cell population.

In some embodiments, fragmentation includes physical fragmentation, including for example, dissection as well as digestion. In some embodiments, the fragmentation is physical fragmentation. In some embodiments, the fragmentation is dissection. In some embodiments, the fragmentation is by digestion. In some embodiments, TILs can be initially cultured from enzymatic tumor digests and tumor fragments obtained from patients. In some embodiments, TILs can be initially cultured from enzymatic tumor digests and tumor fragments obtained from patients prior to genetic modification via TALEN gene editing by introducing sequentially into the TILs nucleic acids, such as mRNAs, encoding TALEN systems targeting PD-1 and TIGIT.

In some embodiments, where the tumor is a solid tumor, the tumor undergoes physical fragmentation after the tumor sample is obtained (as provided in FIG. 10). In some embodiments, the fragmentation occurs before cryopreservation. In some embodiments, the fragmentation occurs after cryopreservation. In some embodiments, the fragmentation occurs after obtaining the tumor and in the absence of any cryopreservation. In some embodiments, the tumor is fragmented and 10, 20, 30, 40, 50, 60, 70, 80, 90, 100 or more fragments or pieces are placed in each container for the first expansion. In some embodiments, the tumor is fragmented and 30 or 40 fragments or pieces are placed in each container for the first expansion. In some embodiments, the tumor is fragmented and 40 fragments or pieces are placed in each container for the first expansion. In some embodiments, the multiple fragments comprise about 4 to about 50 fragments, wherein each fragment has a volume of about 27 mm³. In some embodiments, the multiple fragments comprise about 30 to about 60 fragments with a total volume of about 1300 mm³ to about 1500 mm³. In some embodiments, the multiple fragments comprise about 50 fragments with a total volume of about 1350 mm³. In some embodiments, the multiple fragments comprise about 50 fragments with a total mass of about 1 gram to about 1.5 grams. In some embodiments, the multiple fragments comprise about 4 fragments. In some embodiments, the multiple fragments comprise about to about 100 fragments.

In some embodiments, the TILs are obtained from tumor fragments. In some embodiments, the tumor fragment is obtained by sharp dissection. In some embodiments, the tumor fragment is between about 1 mm³ and 10 mm³. In some embodiments, the tumor fragment is between about 1 mm³ and 8 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³. In some embodiments, the tumors are 1-4 mm×1-4 mm×1-4 mm. In some embodiments, the tumors are 1 mm×1 mm×1 mm. In some embodiments, the tumors are 2 mm×2 mm×2 mm. In some embodiments, the tumors are 3 mm×3 mm×3 mm. In some embodiments, the tumors are 4 mm×4 mm×4 mm.

In some embodiments, the tumors are resected in order to minimize the amount of hemorrhagic, necrotic, and/or fatty tissues on each piece. In some embodiments, the tumors are resected in order to minimize the amount of hemorrhagic tissue on each piece. In some embodiments, the tumors are resected in order to minimize the amount of necrotic tissue on each piece. In some embodiments, the tumors are resected in order to minimize the amount of fatty tissue on each piece.

In some embodiments, the tumor fragmentation is performed in order to maintain the tumor internal structure. In some embodiments, the tumor fragmentation is performed without preforming a sawing motion with a scalpel. In some embodiments, the TILs are obtained from tumor digests. In some embodiments, tumor digests were generated by incubation in enzyme media, for example but not limited to RPMI 1640, 2 mM GlutaMAX, 10 mg/ml gentamicin, 30 U/mL DNase, and 1.0 mg/ml collagenase, followed by mechanical dissociation (GentleMACS, Miltenyi Biotec, Auburn, CA). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

In some embodiments, the harvested cell suspension prior to the first expansion step is called a “primary cell population” or a “freshly harvested” cell population.

In some embodiments, cells can be optionally frozen after sample harvest and stored frozen prior to entry into the expansion described in further detail below, as well as exemplified in FIG. 10.

### C. First Expansion

After dissection or digestion of tumor fragments, for example such as described in FIG. 10, the resulting cells are cultured in serum containing IL-2 under conditions that favor the growth of TILs over tumor and other cells. In some embodiments, the tumor digests are incubated in 2 mL wells in media comprising inactivated human AB serum with 6000 IU/ml of IL-2. This primary cell population is cultured for a period of days, generally from 3 to 14 days, resulting in a bulk TIL population, generally about 1×10⁸ bulk TIL cells, wherein the expanded TILs will be genetically modified via TALEN gene editing by introducing sequentially into the TILs nucleic acids, such as mRNAs, encoding TALEN systems targeting PD-1 and TIGIT. In some embodiments, this primary cell population is cultured for a period of 3 to 9 days, resulting in a bulk TIL population, generally about 1×10⁸ bulk TIL cells, wherein the expanded TILs will be genetically modified via TALEN gene editing by introducing sequentially into the TILs nucleic acids, such as mRNAs, encoding TALEN systems targeting PD-1 and TIGIT. In some embodiments, this primary cell population is cultured for a period of 5 to 7 days, resulting in a bulk TIL population, generally about 1×10⁸ bulk TIL cells, wherein the expanded TILs will be genetically modified via TALEN gene editing by introducing sequentially into the TILs nucleic acids, such as mRNAs, encoding TALEN systems targeting PD-1 and TIGIT. In some embodiments, this primary cell population is cultured for a period of about 7 days, resulting in a bulk TIL population, generally about 1×10⁸ bulk TIL cells, wherein the expanded TILs will be genetically modified via TALEN gene editing by introducing sequentially into the TILs nucleic acids, such as mRNAs, encoding TALEN systems targeting PD-1 and TIGIT.

In embodiments where TIL cultures are initiated in 24-well plates, for example, using Costar 24-well cell culture cluster, flat bottom (Corning Incorporated, Corning, NY, each well can be seeded with 1×10⁶ tumor digest cells or one tumor fragment in 2 mL of complete medium (CM) with IL-2 (6000 IU/mL; Chiron Corp., Emeryville, CA), wherein the expanded TILs will be genetically modified via TALEN gene editing by introducing sequentially into the TILs nucleic acids, such as mRNAs, encoding TALEN systems targeting PD-1 and TIGIT. In some embodiments, the tumor fragment is between about 1 mm³ and 10 mm³.

In some embodiments, the first expansion culture medium is referred to as “CM”, an abbreviation for culture media. In some embodiments, CM for Step B consists of RPMI 1640 with GlutaMAX, supplemented with 10% human AB serum, 25 mM Hepes, and 10 mg/ml gentamicin. In embodiments where cultures are initiated in gas-permeable flasks with a 40 ml capacity and a 10 cm² gas-permeable silicon bottom (for example, G-Rex10; Wilson Wolf Manufacturing, New Brighton, MN, each flask may be loaded with 10-40×10⁶ viable tumor digest cells or 5-30 tumor fragments in 10-40 mL of CM with IL-2. Both the G-Rex10 and 24-well plates may be incubated in a humidified incubator at 37° C. in 5% CO₂ and 5 days after culture initiation, half the media may be removed and replaced with fresh CM and IL-2 and after day 5, half the media may be changed every 2-3 days.

After preparation of the tumor fragments, the resulting cells (i.e., fragments) are cultured in serum containing IL-2 under conditions that favor the growth of TILs over tumor and other cells, wherein the TILs whose growth is favored will be genetically modified via TALEN gene editing by introducing sequentially into the TILs nucleic acids, such as mRNAs, encoding TALEN systems targeting PD-1 and TIGIT. In some embodiments, the tumor digests are incubated in 2 mL wells in media comprising inactivated human AB serum (or, in some cases, as outlined herein, in the presence of aAPC cell population) with 6000 IU/ml of IL-2. This primary cell population is cultured for a period of days, generally from 10 to 14 days, resulting in a bulk TIL population, generally about 1×10⁸ bulk TIL cells. In some embodiments, the growth media during the first expansion comprises IL-2 or a variant thereof. In some embodiments, the IL is recombinant human IL-2 (rhIL-2). In some embodiments the IL-2 stock solution has a specific activity of 20-30×10⁶ IU/mg for a 1 mg vial. In some embodiments the IL-2 stock solution has a specific activity of 20×10⁶ IU/mg for a 1 mg vial. In some embodiments the IL-2 stock solution has a specific activity of 25×10⁶ IU/mg for a 1 mg vial. In some embodiments the IL-2 stock solution has a specific activity of 30×10⁶ IU/mg for a 1 mg vial. In some embodiments, the IL-2 stock solution has a final concentration of 4-8×10⁶ IU/mg of IL-2. In some embodiments, the IL-2 stock solution has a final concentration of 5-7×10⁶ IU/mg of IL-2. In some embodiments, the IL-2 stock solution has a final concentration of 6×10⁶ IU/mg of IL-2. In some embodiments, the first expansion culture media comprises about 10,000 IU/mL of IL-2, about 9,000 IU/mL of IL-2, about 8,000 IU/mL of IL-2, about 7,000 IU/ml of IL-2, about 6000 IU/ml of IL-2 or about 5,000 IU/ml of IL-2. In some embodiments, the first expansion culture media comprises about 9,000 IU/ml of IL-2 to about 5,000 IU/mL of IL-2. In some embodiments, the first expansion culture media comprises about 8,000 IU/ml of IL-2 to about 6,000 IU/mL of IL-2. In some embodiments, the first expansion culture media comprises about 7,000 IU/mL of IL-2 to about 6,000 IU/mL of IL-2. In some embodiments, the first expansion culture media comprises about 6,000 IU/ml of IL-2. In some embodiments, the cell culture medium further comprises IL-2. In some embodiments, the cell culture medium comprises about 3000 IU/ml of IL-2. In some embodiments, the cell culture medium further comprises IL-2. In some embodiments, the cell culture medium comprises about 3000 IU/mL of IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/ml of IL-2. In some embodiments, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, between 1000 and 5000 IU/mL, or about 8000 IU/mL of IL-2.

In some embodiments, the cell culture medium comprises OKT-3 antibody. In some embodiments, the cell culture medium comprises about 30 ng/ml of OKT-3 antibody. In some embodiments, the cell culture medium comprises about 0.1 ng/ml, about 0.5 ng/ml, about 1 ng/ml, about 2.5 ng/ml, about 5 ng/ml, about 7.5 ng/ml, about 10 ng/ml, about 15 ng/ml, about 20 ng/ml, about 25 ng/ml, about 30 ng/ml, about 35 ng/ml, about 40 ng/ml, about 50 ng/ml, about 60 ng/ml, about 70 ng/ml, about 80 ng/ml, about 90 ng/ml, about 100 ng/ml, about 200 ng/mL, about 500 ng/ml, or about 1 μg/mL of OKT-3 antibody. In some embodiments, the cell culture medium comprises between 0.1 ng/ml and 1 ng/mL, between 1 ng/ml and 5 ng/ml, between 5 ng/ml and 10 ng/ml, between 10 ng/ml and 20 ng/ml, between 20 ng/ml and 30 ng/ml, between 30 ng/ml and 40 ng/ml, between 40 ng/ml and 50 ng/ml, and between 50 ng/ml and 100 ng/ml of OKT-3 antibody. In some embodiments, the cell culture medium does not comprise OKT-3 antibody. In some embodiments, the OKT-3 antibody is muromonab (see Table 1).

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 μg/ml and 100 μg/mL. In some embodiments, the TNFRSF agonist is added at a concentration sufficient to achieve a concentration in the cell culture medium of between 20 μg/ml and 40 μg/mL.

In some embodiments, in addition to one or more TNFRSF agonists, the cell culture medium further comprises IL-2 at an initial concentration of about 3000 IU/ml and OKT-3 antibody at an initial concentration of about 30 ng/ml, and wherein the one or more TNFRSF agonists comprises a 4-1BB agonist.

In some embodiments, the first TIL expansion can proceed for 1 day, 2 days, 3 days, 4 days, 5 days, 6 days, 7 days, 8 days, 9 days, 10 days, 11 days, 12 days, 13 days, or 14 days, wherein the expanded TILs will be genetically modified via TALEN gene editing by introducing sequentially into the TILs nucleic acids, such as mRNAs, encoding TALEN systems targeting PD-1 and TIGIT. In some embodiments, the first TIL expansion can proceed for 1 day to 14 days. In some embodiments, the first TIL expansion can proceed for 2 days to 14 days. In some embodiments, the first TIL expansion can proceed for 3 days to 14 days. In some embodiments, the first TIL expansion can proceed for 4 days to 14 days. In some embodiments, the first TIL expansion can proceed for 5 days to 14 days. In some embodiments, the first TIL expansion can proceed for 6 days to 14 days. In some embodiments, the first TIL expansion can proceed for 7 days to 14 days. In some embodiments, the first TIL expansion can proceed for 8 days to 14 days. In some embodiments, the first TIL expansion can proceed for 9 days to 14 days. In some embodiments, the first TIL expansion can proceed for 10 days to 14 days. In some embodiments, the first TIL expansion can proceed for 11 days to 14 days. In some embodiments, the first TIL expansion can proceed for 12 days to 14 days. In some embodiments, the first TIL expansion can proceed for 13 days to 14 days. In some embodiments, the first TIL expansion can proceed for 14 days. In some embodiments, the first TIL expansion can proceed for 1 day to 11 days. In some embodiments, the first TIL expansion can proceed for 2 days to 11 days. In some embodiments, the first TIL expansion can proceed for 3 days to 11 days. In some embodiments, the first TIL expansion can proceed for 4 days to 11 days. In some embodiments, the first TIL expansion can proceed for 5 days to 11 days. In some embodiments, the first TIL expansion can proceed for 6 days to 11 days. In some embodiments, the first TIL expansion can proceed for 7 days to 11 days. In some embodiments, the first TIL expansion can proceed for 8 days to 11 days. In some embodiments, the first TIL expansion can proceed for 9 days to 11 days. In some embodiments, the first TIL expansion can proceed for 10 days to 11 days. In some embodiments, the first TIL expansion can proceed for 11 days. In some embodiments, the first TIL expansion can proceed for 5 days to 7 days. In some embodiments, the first TIL expansion can proceed for 6 days to 7 days. In some embodiments, the first TIL expansion can proceed for 7 days to 12 days. In some embodiments, the first TIL expansion can proceed for 8 days to 12 days. In some embodiments, the first TIL expansion can proceed for 9 days to 12 days. In some embodiments, the first TIL expansion can proceed for 10 days to 12 days. In some embodiments, the first TIL expansion can proceed for 7 days. In some embodiments, the first TIL expansion can proceed for 9 days.

In some embodiments, the first expansion is performed in a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

In some embodiments, the first cell culture medium comprises 6000 IU/ml of IL-2. In some embodiments, the first cell culture medium comprises 3000 IU/ml of IL-2. In some embodiments, the first cell culture medium comprises 2000 IU/ml of IL-2. In some embodiments, the first cell culture medium comprises 1000 IU/ml of IL-2.

### D. Activation

In some embodiments, after the first expansion (pre-REP) step the TILs are activated by adding anti-CD3 agonist and anti-CD28 agonist, such as TransAct, to the culture medium and culturing for about 1 to 3 days, wherein the TILs will be genetically modified via TALEN gene editing by introducing sequentially into the TILs nucleic acids, such as mRNAs, encoding TALEN systems targeting PD-1 and TIGIT.

In some embodiments, the step of activating the second population of TILs (obtained from the first expansion or pre-REP step) can be performed for a period that is, is about, is less than, is more than, 1 day, 2 days, 3 days, or a range that is between any of the above values. For example, in some embodiments, the step of activating the second population of TILs is performed for about 1 day. In some embodiments, the step of activating the second population of TILs is performed for about 2 days. In some embodiments, the step of activating the second population of TILs is performed for about 3 days.

In some embodiments, the step of activating the second population of TILs (obtained from the first expansion or pre-REP step) is performed using anti-CD3 agonist and anti-CD28 agonist, such as TransAct. In some embodiments, the step of activating the second population of TILs is performed using TransAct at 1:10 dilution, at 1:17.5 dilution, at 1:20 dilution, at 1:25 dilution, at 1:30 dilution, at 1:40 dilution, at 1:50 dilution, at 1:60 dilution, at 1:70 dilution, at 1:80 dilution, at 1:90 dilution, or at 1:100 dilution.

In some embodiments, the step of activating the second population of TILs (obtained from the first expansion or pre-REP step) can be performed by adding the anti-CD3 agonist and anti-CD28 agonist, such as TransAct, to the first cell culture medium. In some embodiments, the step of activating the second population of TILs can be performed by replacing the first cell culture medium with a cell culture medium comprising the anti-CD3 agonist and anti-CD28 agonist, such as TransAct.

### E. First and Second TALEN Gene Modification Steps

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease (TALEN). TALE-nucleases are very specific reagents because they need to bind DNA by pairs under obligatory heterodimeric form to obtain dimerization of the cleavage domain Fok-1. Left and right heterodimer members each recognizes a different nucleic sequences of about 14 to 20 bp, together spanning target sequences of 30 to 50 bp overall specificity. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Strategies that enable the rapid assembly of custom TALE arrays include Golden Gate molecular cloning, high-throughput solid-phase assembly, and ligation-independent cloning techniques. Custom-designed TALE arrays are also commercially available through Cellectis Bioresearch (Paris, France), Transposagen Biopharmaceuticals (Lexington, KY, USA), and Life Technologies (Grand Island, NY, USA). Additionally web-based tools, such as TAL Effector-Nucleotide Target 2.0, are available that enable the design of custom TAL effector repeat arrays for desired targets and also provides predicted TAL effector binding sites. See Doyle, et al., Nucleic Acids Research, 2012, Vol. 40, W117-W122. Examples of TALE and TALEN methods suitable for use in the present invention are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

According to some embodiments of the present invention, a TALE method comprises silencing or reducing the expression of one or more genes by inhibiting or preventing transcription of the targeted gene(s). For example, a TALE method may include utilizing KRAB-TALEs, wherein the method comprises fusing a transcriptional Kruppel-associated box (KRAB) domain to a DNA binding domain that targets the gene's transcription start site, leading to the inhibition or prevention of transcription of the gene.

According to other embodiments, a TALE method comprises silencing or reducing the expression of one or more genes by introducing mutations in the targeted gene(s). For example, a TALE method may include fusing a nuclease effector domain, such as Fok1, to the TALE DNA binding domain, resulting in a TALEN. Fok1 is active as a dimer; hence, the method comprises constructing pairs of TALENs to position the Fok1 nuclease domains to adjacent genomic target sites, where they introduce DNA double strand breaks. A double strand break may be completed following correct positioning and dimerization of Fok1. Once the double strand break is introduced, DNA repair can be achieved via two different mechanisms: the high-fidelity homologous recombination repair (HRR) (also known as homology-directed repair or HDR) or the error-prone non-homologous end joining (NHEJ). Repair of double strand breaks via NHEJ preferably results in DNA target site deletions, insertions or substitutions, i.e., NHEJ typically leads to the introduction of small insertions and deletions at the site of the break, often inducing frameshifts that knockout gene function. According to particular embodiments, the TALEN pairs are targeted to the most 5′ exons of the genes, promoting early frame shift mutations or premature stop codons. The genetic mutation(s) introduced by TALEN are generally permanent. Thus, according to some embodiments, the method comprises silencing or reducing expression of a target gene by utilizing dimerized TALENs to induce a site-specific double strand break that is repaired via error-prone NHEJ, leading to one or more mutations in the targeted gene.

According to other embodiments, a TALEN that is a hybrid protein derived from FokI and AvrXa7, as disclosed in U.S. Patent Publication No. 2011/0201118, may be used in accordance with embodiments of the present invention. This TALEN retains recognition specificity for target nucleotides of AvrXa7 and the double-stranded DNA cleaving activity of FokI. The same methods can be used to prepare other TALEN having different recognition specificity. For example, compact TALENs may be generated by engineering a core TALE scaffold having different sets of RVDs to change the DNA binding specificity and target a specific single dsDNA target sequence. See U.S. Patent Publication No. 2013/0117869. A selection of catalytic domains can be attached to the scaffold to effect DNA processing, which may be engineered to ensure that the catalytic domain is capable of processing DNA near the single dsDNA target sequence when fused to the core TALE scaffold. A peptide linker may also be engineered to fuse the catalytic domain to the scaffold to create a compact TALEN made of a single polypeptide chain that does not require dimerization to target a specific single dsDNA sequence. A core TALE scaffold may also be modified by fusing a catalytic domain, which may be a TAL monomer, to its N-terminus, allowing for the possibility that this catalytic domain might interact with another catalytic domain fused to another TAL monomer, thereby creating a catalytic entity likely to process DNA in the proximity of the target sequences. See U.S. Patent Publication No. 2015/0203871. This architecture allows only one DNA strand to be targeted, which is not an option for classical TALEN architectures.

In some embodiments, the activation step is followed by two steps of genetically modifying TILs by introducing into the TILs nucleic acids, such as mRNAs, encoding two TALEN systems that target PD-1 and TIGIT. In some embodiments, the TALEN gene modification steps are performed by genetically modifying the TILs obtained from the activation step by sequential electroporation of TILs with nucleic acids, such as mRNAs, encoding the two TALEN systems that target PD-1 and TIGIT. In some embodiments, the TALEN gene modification steps are performed by genetically modifying the TILs obtained from the activation step by electroporation of TILs with nucleic acids, such as mRNAs, encoding a TALEN system that targets PD-1, followed by electroporation of TILs with nucleic acids, such as mRNAs, encoding a TALEN system that targets TIGIT. In some embodiments, the TALEN gene modification steps are performed by genetically modifying the TILs obtained from the activation step by electroporation of TILs with nucleic acids, such as mRNAs, encoding a TALEN system that targets TIGIT, followed by electroporation of TILs with nucleic acids, such as mRNAs, encoding a TALEN system that targets PD-1.

In some embodiments, the activation step is followed by two steps of genetically modifying TILs by introducing into the TILs nucleic acids, such as mRNAs, encoding two TALEN systems that target PD-1 and LAG3. In some embodiments, the TALEN gene modification steps are performed by genetically modifying the TILs obtained from the activation step by sequential electroporation of TILs with nucleic acids, such as mRNAs, encoding the two TALEN systems that target PD-1 and LAG3. In some embodiments, the TALEN gene modification steps are performed by genetically modifying the TILs obtained from the activation step by electroporation of TILs with nucleic acids, such as mRNAs, encoding a TALEN system that targets PD-1, followed by electroporation of TILs with nucleic acids, such as mRNAs, encoding a TALEN system that targets LAG3. In some embodiments, the TALEN gene modification steps are performed by genetically modifying the TILs obtained from the activation step by electroporation of TILs with nucleic acids, such as mRNAs, encoding a TALEN system that targets LAG3, followed by electroporation of TILs with nucleic acids, such as mRNAs, encoding a TALEN system that targets PD-1.

Embodiments disclosed herein further provide the polynucleotide sequences encoding the TALEN heterodimers (also referred to as half-TALEN), in particular an mRNA sequence encoding a TALEN protein that targets PD-1 and/or TIGIT, a TALEN protein that targets PD-1 and/or TIGIT, a DNA sequence encoding an mRNA encoding a TALEN protein that targets PD-1 and/or TIGIT, etc.

Embodiments disclosed herein further provide the polynucleotide sequences encoding the TALEN heterodimers (also referred to as half-TALEN), in particular an mRNA sequence encoding a TALEN protein that targets PD-1 and/or LAG3, a TALEN protein that targets PD-1 and/or LAG3, a DNA sequence encoding an mRNA encoding a TALEN protein that targets PD-1 and/or LAG3, etc.

In some embodiments, the mRNA sequence encoding a TALEN system that targets PD-1, TIGIT, and/or LAG3 may be produced in vitro. In some embodiments, the TALEN mRNA may be transcribed from linearized plasmid DNA encoding each TALEN arm of interest by an RNA polymerase. In some embodiments, the invention provides an in vitro transcription process comprising a mixture of DNA template, RNA polymerase, and nucleotide triphosphates (NTPs) with magnesium-containing buffer, RNase inhibitor, and inorganic pyrophosphatase.

In some embodiments, the invention provides a method for post-transcriptional modification of the transcribed mRNA to add a cap by further treating the mRNA with an enzyme to form a 5′ capped mRNA. See Ensinger, et al., PNAS, 1975, 72 (7) 2525-2529; Moss, et al., Virology, 1976, 72 (2), 341-351, the contents of which are herein incorporated by reference in their entireties. Alternatively, capped transcripts can be produced by using a cap analog during the in vitro transcription reaction. See Ishikawa, et al., Nucl. Acids Symp. Series, 2009, 53, 129-131; Sikorski, et al., Nucl. Acid Res., 2020, 48 (4), 1607-1626; Stepinski, et al., RNA, 2001, 7 (10), 1486-1495, the contents of which are herein incorporated by reference in their entireties. In some embodiments, the invention provides a process for in vitro transcription in which 5′ capped mRNA transcripts can be produced by using a cap analog during the in vitro transcription reaction, without any post-transcriptional modification. In some embodiments, the mRNA sequence encoding a TALEN system that targets PD-1 or TIGIT may be produced in vitro using the CleanCap® AG technology by TriLink Biotechnologies, which is described in Henderson, et al., Current Protocols, 2021, 1, e39. doi: 10.1002/cpz1.39; and PCT Patent Publication No. WO 2017053297 A1, the contents of which are herein incorporated by reference in their entireties. In some embodiments, the mRNA sequence encoding a TALEN system that targets PD-1 or TIGIT may be transcribed from linearized plasmid DNA encoding each TALEN arm of interest using the “Basic Protocol 1: IVT WITH CleanCap” described in Henderson, et al., supra.

In some embodiments, the invention provides a DNA template for transcription of an mRNA comprising a sequence encoding a TALEN system that targets PD-1, TIGIT, or LAG3, and further comprising a 5′ un-transcribed region (UTR) compatible with the CleanCap® AG technology having the sequence of AGCTAGCGCCGCCACC (SEQ ID NO: 30). In some embodiments, the DNA template for the mRNA sequence encoding a TALEN system that targets PD-1, TIGIT, or LAG3 comprises a T7 RNA polymerase promotor sequence of TAATACGACTCACTATA (SEQ ID NO: 31) before the 5′ UTR.

In some embodiments, the invention provides a process for preparing expanded tumor infiltrating lymphocytes (TILs) having reduced expression of PD-1 and TIGIT, or reduced expression of PD-1 and LAG3, using an mRNA encoding the TALEN system that targets PD-1, TIGIT, or LAG3 that is introduced at about 0.1-20 μg mRNA/million cells. For example, the mRNA encoding the TALEN system that targets PD-1, TIGIT, or LAG3 is introduced at about 0.1 μg mRNA/million cells, about 0.2 μg mRNA/million cells, about 0.3 μg mRNA/million cells, about 0.4 μg mRNA/million cells, about 0.5 μg mRNA/million cells, about 0.6 μg mRNA/million cells, about 0.7 μg mRNA/million cells, about 0.8 μg mRNA/million cells, about 0.9 μg mRNA/million cells, about 1 μg mRNA/million cells, about 1.5 μg mRNA/million cells, about 2 μg mRNA/million cells, about 3 μg mRNA/million cells, about 4 μg mRNA/million cells, about 5 μg mRNA/million cells, about 6 μg mRNA/million cells, about 7 μg mRNA/million cells, about 8 μg mRNA/million cells, about 9 μg mRNA/million cells, about 10 μg mRNA/million cells, or about 20 μg mRNA/million cells. In some embodiments, the mRNA encoding the TALEN system that targets PD-1, TIGIT, or LAG3 is introduced at about 0.1-10 μg mRNA/million cells. In some embodiments, the mRNA encoding the TALEN system that targets PD-1, TIGIT, or LAG3 is introduced at about 0.1-4 μg mRNA/million cells. In some embodiments, the mRNA encoding the TALEN system that targets PD-1, TIGIT, or LAG3 is introduced at about 0.5-4 μg mRNA/million cells. In some embodiments, the mRNA encoding the TALEN system that targets PD-1, TIGIT, or LAG3 is introduced at about 0.5 μg mRNA/million cells. In some embodiments, the mRNA encoding the TALEN system that targets PD-1, TIGIT, or LAG3 is introduced at about 1 μg mRNA/million cells. In some embodiments, the mRNA encoding the TALEN system that targets PD-1, TIGIT, or LAG3 is introduced at about 2 μg mRNA/million cells. In some embodiments, the mRNA encoding the TALEN system that targets PD-1, TIGIT, or LAG3 is introduced at about 4 μg mRNA/million cells.

In some embodiments, the invention provides a process for preparing expanded tumor infiltrating lymphocytes (TILs) having reduced expression of PD-1 and TIGIT using an mRNA encoding the TALEN system that targets PD-1 comprising the amino acid sequences of SEQ ID NOs: 14 and 16 that is introduced at about 10 μg/ml or 12.5 μg/mL mRNA per TALEN arm and an mRNA encoding the TALEN system that targets TIGIT comprising the amino acid sequences of SEQ ID NOs: 20 and 22 that is introduced at about 40 μg/mL or 50 μg/ml mRNA per TALEN arm. In some embodiments, the invention provides a process for preparing expanded tumor infiltrating lymphocytes (TILs) having reduced expression of PD-1 and TIGIT using an mRNA encoding the TALEN system that targets PD-1 comprising the amino acid sequences of SEQ ID NOs: 14 and 16 that is introduced at about 10 g/mL mRNA per TALEN arm and an mRNA encoding the TALEN system that targets TIGIT comprising the amino acid sequences of SEQ ID NOs: 20 and 22 that is introduced at about 40 μg/mL mRNA per TALEN arm. In some embodiments, the invention provides a process for preparing expanded tumor infiltrating lymphocytes (TILs) having reduced expression of PD-1 and TIGIT using an mRNA encoding the TALEN system that targets PD-1 comprising the amino acid sequences of SEQ ID NOs: 14 and 16 that is introduced at about 10 μg/ml mRNA per TALEN arm and an mRNA encoding the TALEN system that targets TIGIT comprising the amino acid sequences of SEQ ID NOs: 20 and 22 that is introduced at about 50 μg/mL mRNA per TALEN arm. In some embodiments, the invention provides a process for preparing expanded tumor infiltrating lymphocytes (TILs) having reduced expression of PD-1 and TIGIT using an mRNA encoding the TALEN system that targets PD-1 comprising the amino acid sequences of SEQ ID NOs: 14 and 16 that is introduced at about 12.5 μg/mL mRNA per TALEN arm and an mRNA encoding the TALEN system that targets TIGIT comprising the amino acid sequences of SEQ ID NOs: 20 and 22 that is introduced at about 40 μg/ml mRNA per TALEN arm. In some embodiments, the invention provides a process for preparing expanded tumor infiltrating lymphocytes (TILs) having reduced expression of PD-1 and TIGIT using an mRNA encoding the TALEN system that targets PD-1 comprising the amino acid sequences of SEQ ID NOs: 14 and 16 that is introduced at about 12.5 μg/ml mRNA per TALEN arm and an mRNA encoding the TALEN system that targets TIGIT comprising the amino acid sequences of SEQ ID NOs: 20 and 22 that is introduced at about 50 μg/ml mRNA per TALEN arm.

Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In some embodiments, the electroporation method is a sterile electroporation method. In some embodiments, the electroporation method is a pulsed electroporation method. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses 3% in some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse amplitude. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse width. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to induce pore formation in the TILs, comprising the step of applying a sequence of at least three DC electrical pulses, having field strengths equal to or greater than 100 V/cm, to TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the TILs is maintained. In some embodiments, a method of genetically modifying a population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In some embodiments, a method of genetically modifying a population of TILs includes the step of liposomal transfection. Liposomal transfection methods, such as methods that employ a 1:1 (w/w) liposome formulation of the cationic lipid N-[1-(2,3-dioleyloxy) propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In some embodiments, a method of genetically modifying a population of TILs includes the step of transfection using methods described in U.S. Pat. Nos. 5,766,902; 6,025,337; 6,410,517; 6,475,994; and 7,189,705; the disclosures of each of which are incorporated by reference herein.

In some embodiments of the present invention, electroporation is used for delivery of the desired TALEN-encoding nucleic acid, including TALEN-encoding RNAs and/or DNAs. In some embodiments of the present invention, the electroporation system is a flow electroporation system. An example of a suitable flow electroporation system suitable for use with some embodiments of the present invention is the commercially-available MaxCyte STX system. There are several alternative commercially-available electroporation instruments which may be suitable for use with the present invention, such as the CTS Xenon Electroporation System or the Neon Transfection System available from Thermo-Fisher, the AgilePulse system or ECM 830 available from BTX-Harvard Apparatus, Cellaxess Elektra (Cellectricon), Nucleofector (Lonza/Amaxa), GenePulser MXcell (BIORAD), iPorator-96 (Primax) or siPORTer96 (Ambion). In some embodiments of the present invention, the electroporation system forms a closed, sterile system with the remainder of the TIL expansion method. In some embodiments of the present invention, the electroporation system is a pulsed electroporation system as described herein, and forms a closed, sterile system with the remainder of the TIL expansion method.

### 1. PD-1

One of the most studied targets for the induction of checkpoint blockade is the programmed death receptor (PD1 or PD-1, also known as PDCD1), a member of the CD28 super family of T-cell regulators. Its ligands, PD-L1 and PD-L2, are expressed on a variety of tumor cells, including melanoma. The interaction of PD-1 with PD-L1 inhibits T-cell effector function, results in T-cell exhaustion in the setting of chronic stimulation, and induces T-cell apoptosis in the tumor microenvironment. PD-1 may also play a role in tumor-specific escape from immune surveillance.

The expression of PD-1 in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein, wherein the method comprises gene-editing at least a portion of the TILs by silencing or repressing the expression of PD-1. As described in more detail below, the gene-editing process may involve the use of a programmable nuclease that mediates the generation of a double-strand or single-strand break at an immune checkpoint gene, such as PD-1. For example, a TALEN method may be used to silence or reduce the expression of PD-1 in the TILs.

According to particular embodiments, the invention provides a method for expanding the genetically modified tumor infiltrating lymphocytes (TILs) into a therapeutic population of TILs, where the genetically modified TILs are produced by introducing into the TILs nucleic acids, optionally mRNAs, encoding one or more TALE-nucleases able to selectively inactivate by DNA cleavage a gene encoding TIGIT, wherein the one or more TALE-nucleases comprise a TALE-nuclease that is directed against one of the gene target sequences of PD-1 comprising the nucleic acid sequence of SEQ ID NO: 18, and wherein the method optionally further comprises TALEN gene-editing at least a portion of the TILs by silencing or repressing the expression of TIGIT. For example, this TALE method can be used to silence or reduce the expression of TIGIT in the TILs, in addition to PD-1. In some embodiments, the TALENs targeting the PD-1 gene are those described in WO 2013/176915 A1, WO 2014/184744 A1, WO 2014/184741 A1, WO 2018/007263 A1, and WO 2018/073391 A1 including any of the PD-1 TALENs described in Table 10 on pages 62-63 of WO 2013/176915 A1, any of the PD-1 TALENs described in Table 11 on page 78 of WO 2014/184744 A1, any of the PD-1 TALENs described in Table 11 on page 75 of WO 2014/184741 A1, any of the PD-1 TALENs described in Table 3 on pages 48-52 of WO 2018/007263 A1, and any of the PD-1 TALENs described in Table 4 on pages 62-68 and/or in Table 5 on pages 73-99 of WO 2018/073391 A1, the contents of which are incorporated by reference in their entireties.

Examples of TALE-nucleases, and TALE-nuclease-encoding sequences, targeting the PD-1 gene are provided in the following Table 3. According to particular embodiments, TALE-nucleases according to the invention recognize and cleave a target sequence of SEQ ID NO: 18. According to particular embodiments, TALE-nucleases according to the invention comprise the amino acid sequences of SEQ ID NOs: 14 and 16. According to particular embodiments, TALE-nucleases according to the invention are encoded by the nucleotide sequences of SEQ ID NOs: 13 and 15.

TABLE 3
PD-1 KO TALE-nucleases and sequences of TALE-nuclease cleavage site in the
human PD-1 gene

| Sequence | Ref. | |
|---|---|---|
| name | Sequences | Polynucleotide or polypeptide sequences |
| Left PD-1 | SEQ ID NO: | ATGGGCGATCCTAAAAAGAAACGTAAGGTCATCGATATCGCCGATCTACGC |
| TALEN | 13 | ACGCTCGGCTACAGCCAGCAGCAACAGGAGAAGATCAAACCGAAGGTTCGT |
| NT | | TCGACAGTGGCGCAGCACCACGAGGCACTGGTCGGCCACGGGTTTACACAC |
| sequence | | GCGCACATCGTTGCGTTAAGCCAACACCCGGCAGCGTTAGGGACCGTCGCT |
| | | GTCAAGTATCAGGACATGATCGCAGCGTTGCCAGAGGCGACACACGAAGCG |
| | | ATCGTTGGCGTCGGCAAACAGTGGTCCGGCGCACGCGCTCTGGAGGCCTTG |
| | | CTCACGGTGGCGGGAGAGTTGAGAGGTCCACCGTTACAGTTGGACACAGGC |
| | | CAACTTCTCAAGATTGCAAAACGTGGCGGCGTGACCGCAGTGGAGGCAGTG |
| | | CATGCATGGCGCAATGCACTGACGGGTGCCCCGCTCAACTTGACCCCCGAG |
| | | CAAGTGGTGGCTATCGCTTCCAAGCTGGGGGGAAAGCAGGCCCTGGAGACC |
| | | GTCCAGGCCCTTCTCCCAGTGCTTTGCCAGGCTCACGGACTGACCCCTGAA |
| | | CAGGTGGTGGCAATTGCCTCACACGACGGGGGCAAGCAGGCACTGGAGACT |
| | | GTCCAGCGGCTGCTGCCTGTCCTCTGCCAGGCCCACGGACTCACTCCTGAG |
| | | CAGGTCGTGGCCATTGCCAGCCACGATGGGGGCAAACAGGCTCTGGAGACC |
| | | GTGCAGCGCCTCCTCCCAGTGCTGTGCCAGGCTCATGGGCTGACCCCACAG |
| | | CAGGTCGTCGCCATTGCCAGTAACGGCGGGGGGAAGCAGGCCCTCGAAACA |
| | | GTGCAGAGGCTGCTGCCCGTCTTGTGCCAAGCACACGGCCTGACACCCGAG |
| | | CAGGTGGTGGCCATCGCCTCTCATGACGGCGGCAAGCAGGCCCTTGAGACA |
| | | GTGCAGAGACTGTTGCCCGTGTTGTGTCAGGCCCACGGGTTGACACCCCAG |
| | | CAGGTGGTCGCCATCGCCAGCAATGGCGGGGGAAAGCAGGCCCTTGAGACC |
| | | GTGCAGCGGTTGCTTCCAGTGTTGTGCCAGGCACACGGACTGACCCCTCAA |
| | | CAGGTGGTCGCAATCGCCAGCTACAAGGGCGGAAAGCAGGCTCTGGAGACA |
| | | GTGCAGCGCCTCCTGCCCGTGCTGTGTCAGGCTCACGGACTGACACCACAG |
| | | CAGGTGGTCGCCATCGCCAGTAACGGGGGCGGCAAGCAGGCTTTGGAGACC |
| | | GTCCAGAGACTCCTCCCCGTCCTTTGCCAGGCCCACGGGTTGACACCTCAG |
| | | CAGGTCGTCGCCATTGCCTCCAACAACGGGGGCAAGCAGGCCCTCGAAACT |
| | | GTGCAGAGGCTGCTGCCTGTGCTGTGCCAGGCTCATGGGCTGACACCCCAG |
| | | CAGGTGGTGGCCATTGCCTCTAACAACGGCGGCAAACAGGCACTGGAGACC |
| | | GTGCAAAGGCTGCTGCCCGTCCTCTGCCAAGCCCACGGGCTCACTCCACAG |
| | | CAGGTCGTGGCCATCGCCTCAAACAATGGCGGGAAGCAGGCCCTGGAGACT |
| | | GTGCAAAGGCTGCTCCCTGTGCTCTGCCAGGCACACGGACTGACCCCTCAG |
| | | CAGGTGGTGGCAATCGCTTCCAACAACGGGGGAAAGCAGGCCCTCGAAACC |
| | | GTGCAGCGCCTCCTCCCAGTGCTGTGCCAGGCACATGGCCTCACACCCGAG |
| | | CAAGTGGTGGCTATCGCCAGCCACGACGGAGGGAAGCAGGCTCTGGAGACC |
| | | GTGCAGAGGCTGCTGCCTGTCCTGTGCCAGGCCCACGGGCTTACTCCAGAG |
| | | CAGGTCGTCGCCATCGCCAGTCATGATGGGGGGAAGCAGGCCCTTGAGACA |
| | | GTCCAGCGGCTGCTGCCAGTCCTTTGCCAGGCTCACGGCTTGACTCCCGAG |
| | | CAGGTCGTGGCCATTGCCTCAAACATTGGGGGCAAACAGGCCCTGGAGACA |
| | | GTGCAGGCCCTGCTGCCCGTGTTGTGTCAGGCCCACGGCTTGACACCCCAG |
| | | CAGGTGGTCGCCATTGCCTCTAATGGCGGCGGGAGACCCGCCTTGGAGAGC |
| | | ATTGTTGCCCAGTTATCTCGCCCTGATCCGGCGTTGGCCGCGTTGACCAAC |
| | | GACCACCTCGTCGCCTTGGCCTGCCTCGGCGGGCGTCCTGCGCTGGATGCA |
| | | GTGAAAAAGGGATTGGGGGATCCTATCAGCCGTTCCCAGCTGGTGAAGTCC |
| | | GAGCTGGAGGAGAAGAAATCCGAGTTGAGGCACAAGCTGAAGTACGTGCCC |
| | | CACGAGTACATCGAGCTGATCGAGATCGCCCGGAACAGCACCCAGGACCGT |
| | | ATCCTGGAGATGAAGGTGATGGAGTTCTTCATGAAGGTGTACGGCTACAGG |
| | | GGCAAGCACCTGGGCGGCTCCAGGAAGCCCGACGGCGCCATCTACACCGTG |
| | | GGCTCCCCCATCGACTACGGCGTGATCGTGGACACCAAGGCCTACTCCGGC |
| | | GGCTACAACCTGCCCATCGGCCAGGCCGACGAAATGCAGAGGTACGTGGAG |
| | | GAGAACCAGACCAGGAACAAGCACATCAACCCCAACGAGTGGTGGAAGGTG |
| | | TACCCCTCCAGCGTGACCGAGTTCAAGTTCCTGTTCGTGTCCGGCCACTTC |
| | | AAGGGCAACTACAAGGCCCAGCTGACCAGGCTGAACCACATCACCAACTGC |
| | | AACGGCGCCGTGCTGTCCGTGGAGGAGCTCCTGATCGGCGGCGAGATGATC |
| | | AAGGCCGGCACCCTGACCCTGGAGGAGGTGAGGAGGAAGTTCAACAACGGC |
| | | GAGATCAACTTCGCGGCCGACTGATAA |
| Left PD-1 | SEQ ID NO: | MGDPKKKRKVIDIADLRTLGYSQQQQEKIKPKVRSTVAQHHEALVGHGFTH |
| TALEN | 14 | AHIVALSQHPAALGTVAVKYQDMIAALPEATHEAIVGVGKOWSGARALEAL |
| Amino acid | | LTVAGELRGPPLQLDTGQLLKIAKRGGVTAVEAVHAWRNALTGAPLNLTPE |
| sequence | | QVVAIASKLGGKQALETVQALLPVLCQAHGLTPEQVVAIASHDGGKQALET |
| | | VQRLLPVLCQAHGLTPEQVVAIASHDGGKQALETVORLLPVLCQAHGLTPQ |
| | | QVVAIASNGGGKQALETVQRLLPVLCQAHGLTPEQVVAIASHDGGKQALET |
| | | VQRLLPVLCQAHGLTPQQVVAIASNGGGKQALETVORLLPVLCQAHGLTPQ |
| | | QVVAIASYKGGKQALETVQRLLPVLCQAHGLTPQQVVAIASNGGGKQALET |
| | | VQRLLPVLCQAHGLTPQQVVAIASNNGGKQALETVORLLPVLCQAHGLTPQ |
| | | QVVAIASNNGGKQALETVORLLPVLCQAHGLTPQQVVAIASNNGGKQALET |
| | | VQRLLPVLCQAHGLTPQQVVAIASNNGGKQALETVQRLLPVLCQAHGLTPE |
| | | QVVAIASHDGGKQALETVORLLPVLCQAHGLTPEQVVAIASHDGGKQALET |
| | | VQRLLPVLCQAHGLTPEQVVAIASNIGGKQALETVQALLPVLCQAHGLTPQ |
| | | QVVAIASNGGGRPALESIVAQLSRPDPALAALTNDHLVALACLGGRPALDA |
| | | VKKGLGDPISRSQLVKSELEEKKSELRHKLKYVPHEYIELIEIARNSTQDR |
| | | ILEMKVMEFFMKVYGYRGKHLGGSRKPDGAIYTVGSPIDYGVIVDTKAYSG |
| | | GYNLPIGQADEMQRYVEENQTRNKHINPNEWWKVYPSSVTEFKFLFVSGHF |
| | | KGNYKAQLTRLNHITNCNGAVLSVEELLIGGEMIKAGTLTLEEVRRKFNNG |
| | | EINFAAD |
| Right PD-1 | SEQ ID NO: | ATGGGCGATCCTAAAAAGAAACGTAAGGTCATCGATATCGCCGATCTACGC |
| TALEN | 15 | ACGCTCGGCTACAGCCAGCAGCAACAGGAGAAGATCAAACCGAAGGTTCGT |
| NT | | TCGACAGTGGCGCAGCACCACGAGGCACTGGTCGGCCACGGGTTTACACAC |
| sequence | | GCGCACATCGTTGCGTTAAGCCAACACCCGGCAGCGTTAGGGACCGTCGCT |
| | | GTCAAGTATCAGGACATGATCGCAGCGTTGCCAGAGGCGACACACGAAGCG |
| | | ATCGTTGGCGTCGGCAAACAGTGGTCCGGCGCACGCGCTCTGGAGGCCTTG |
| | | CTCACGGTGGCGGGAGAGTTGAGAGGTCCACCGTTACAGTTGGACACAGGC |
| | | CAACTTCTCAAGATTGCAAAACGTGGCGGCGTGACCGCAGTGGAGGCAGTG |
| | | CATGCATGGCGCAATGCACTGACGGGTGCCCCGCTCAACTTGACCCCCGAG |
| | | CAAGTCGTCGCAATCGCCAGCCATGATGGAGGGAAGCAAGCCCTCGAAACC |
| | | GTGCAGCGGTTGCTTCCTGTGCTCTGCCAGGCCCACGGCCTTACCCCTCAG |
| | | CAGGTGGTGGCCATCGCAAGTAACGGAGGAGGAAAGCAAGCCTTGGAGACA |
| | | GTGCAGCGCCTGTTGCCCGTGCTGTGCCAGGCACACGGCCTCACACCAGAG |
| | | CAGGTCGTGGCCATTGCCTCCCATGACGGGGGGAAACAGGCTCTGGAGACC |
| | | GTCCAGAGGCTGCTGCCCGTCCTCTGTCAAGCTCACGGCCTGACTCCCCAA |
| | | CAAGTGGTCGCCATCGCCTCTAATGGCGGCGGGAAGCAGGCACTGGAAACA |
| | | GTGCAGAGACTGCTCCCTGTGCTTTGCCAAGCTCATGGGTTGACCCCCCAA |
| | | CAGGTCGTCGCTATTGCCTCAAACGGGGGGGGCAAGCAGGCCCTTGAGACT |
| | | GTGCAGAGGCTGTTGCCAGTGCTGTGTCAGGCTCACGGGCTCACTCCACAA |
| | | CAGGTGGTCGCAATTGCCAGCAACGGCGGCGGAAAGCAAGCTCTTGAAACC |
| | | GTGCAACGCCTCCTGCCCGTGCTCTGTCAGGCTCATGGCCTGACACCACAA |
| | | CAAGTCGTGGCCATCGCCAGTAATAATGGCGGGAAACAGGCTCTTGAGACC |
| | | GTCCAGAGGCTGCTCCCAGTGCTCTGCCAGGCACACGGGCTGACCCCCGAG |
| | | CAGGTGGTGGCTATCGCCAGCAATATTGGGGGCAAGCAGGCCCTGGAAACA |
| | | GTCCAGGCCCTGCTGCCAGTGCTTTGCCAGGCTCACGGGCTCACTCCCCAG |
| | | CAGGTCGTGGCAATCGCCTCCAACGGCGGAGGGAAGCAGGCTCTGGAGACC |
| | | GTGCAGAGACTGCTGCCCGTCTTGTGCCAGGCCCACGGACTCACACCTGAA |
| | | CAGGTCGTCGCCATTGCCTCTCACGATGGGGGCAAACAAGCCCTGGAGACA |
| | | GTGCAGCGGCTGTTGCCTGTGTTGTGCCAAGCCCACGGCTTGACTCCTCAA |
| | | CAAGTGGTCGCCATCGCCTCAAATGGCGGCGGAAAACAAGCTCTGGAGACA |
| | | GTGCAGAGGTTGCTGCCCGTCCTCTGCCAAGCCCACGGCCTGACTCCCCAA |
| | | CAGGTCGTCGCCATTGCCAGCAACAACGGAGGAAAGCAGGCTCTCGAAACT |
| | | GTGCAGCGGCTGCTTCCTGTGCTGTGTCAGGCTCATGGGCTGACCCCCGAG |
| | | CAAGTGGTGGCTATTGCCTCTAATGGAGGCAAGCAAGCCCTTGAGACAGTC |
| | | CAGAGGCTGTTGCCAGTGCTGTGCCAGGCCCACGGGCTCACACCCCAGCAG |
| | | GTGGTCGCCATCGCCAGTAACAACGGGGGCAAACAGGCATTGGAAACCGTC |
| | | CAGCGCCTGCTTCCAGTGCTCTGCCAGGCACACGGACTGACACCCGAACAG |
| | | GTGGTGGCCATTGCATCCCATGATGGGGGCAAGCAGGCCCTGGAGACCGTG |
| | | CAGAGACTCCTGCCAGTGTTGTGCCAAGCTCACGGCCTCACCCCTCAGCAA |
| | | GTCGTGGCCATCGCCTCAAACGGGGGGGGCCGGCCTGCACTGGAGAGCATT |
| | | GTTGCCCAGTTATCTCGCCCTGATCCGGCGTTGGCCGCGTTGACCAACGAC |
| | | CACCTCGTCGCCTTGGCCTGCCTCGGCGGGCGTCCTGCGCTGGATGCAGTG |
| | | AAAAAGGGATTGGGGGATCCTATCAGCCGTTCCCAGCTGGTGAAGTCCGAG |
| | | CTGGAGGAGAAGAAATCCGAGTTGAGGCACAAGCTGAAGTACGTGCCCCAC |
| | | GAGTACATCGAGCTGATCGAGATCGCCCGGAACAGCACCCAGGACCGTATC |
| | | CTGGAGATGAAGGTGATGGAGTTCTTCATGAAGGTGTACGGCTACAGGGGC |
| | | AAGCACCTGGGCGGCTCCAGGAAGCCCGACGGCGCCATCTACACCGTGGGC |
| | | TCCCCCATCGACTACGGCGTGATCGTGGACACCAAGGCCTACTCCGGCGGC |
| | | TACAACCTGCCCATCGGCCAGGCCGACGAAATGCAGAGGTACGTGGAGGAG |
| | | AACCAGACCAGGAACAAGCACATCAACCCCAACGAGTGGTGGAAGGTGTAC |
| | | CCCTCCAGCGTGACCGAGTTCAAGTTCCTGTTCGTGTCCGGCCACTTCAAG |
| | | GGCAACTACAAGGCCCAGCTGACCAGGCTGAACCACATCACCAACTGCAAC |
| | | GGCGCCGTGCTGTCCGTGGAGGAGCTCCTGATCGGCGGCGAGATGATCAAG |
| | | GCCGGCACCCTGACCCTGGAGGAGGTGAGGAGGAAGTTCAACAACGGCGAG |
| | | ATCAACTTCGCGGCCGACTGATAA |
| Right PD-1 | SEQ ID NO: | MGDPKKKRKVIDIADLRTLGYSQQQQEKIKPKVRSTVAQHHEALVGHGFTH |
| TALEN | 16 | AHIVALSQHPAALGTVAVKYQDMIAALPEATHEAIVGVGKOWSGARALEAL |
| Amino acid | | LTVAGELRGPPLQLDTGQLLKIAKRGGVTAVEAVHAWRNALTGAPLNLTPE |
| sequence | | QVVAIASHDGGKQALETVQRLLPVLCQAHGLTPQQVVAIASNGGGKQALET |
| | | VQRLLPVLCQAHGLTPEQVVAIASHDGGKQALETVORLLPVLCQAHGLTPQ |
| | | QVVAIASNGGGKQALETVQRLLPVLCQAHGLTPQQVVAIASNGGGKQALET |
| | | VQRLLPVLCQAHGLTPQQVVAIASNGGGKQALETVORLLPVLCQAHGLTPQ |
| | | QVVAIASNNGGKQALETVORLLPVLCQAHGLTPEQVVAIASNIGGKQALET |
| | | VQALLPVLCQAHGLTPQQVVAIASNGGGKQALETVORLLPVLCQAHGLTPE |
| | | QVVAIASHDGGKQALETVQRLLPVLCQAHGLTPQQVVAIASNGGGKQALET |
| | | VQRLLPVLCQAHGLTPQQVVAIASNNGGKQALETVORLLPVLCQAHGLTPE |
| | | QVVAIASNGGKQALETVQRLLPVLCQAHGLTPQQVVAIASNNGGKQALETV |
| | | QRLLPVLCQAHGLTPEQVVAIASHDGGKQALETVORLLPVLCQAHGLTPQQ |
| | | VVAIASNGGGRPALESIVAQLSRPDPALAALTNDHLVALACLGGRPALDAV |
| | | KKGLGDPISRSQLVKSELEEKKSELRHKLKYVPHEYIELIEIARNSTQDRI |
| | | LEMKVMEFFMKVYGYRGKHLGGSRKPDGAIYTVGSPIDYGVIVDTKAYSGG |
| | | YNLPIGQADEMQRYVEENQTRNKHINPNEWWKVYPSSVTEFKFLFVSGHFK |
| | | GNYKAQLTRLNHITNCNGAVLSVEELLIGGEMIKAGTLTLEEVRRKFNNGE |
| | | INFAAD |
| Left PD-1 | SEQ ID NO: | ATGGGCGATCCTAAAAAGAAACGTAAGGTCATCGATTACCCATACGATGTT |
| TALEN | 17 | CCAGATTACGCTATCGATATCGCCGATCTACGCACGCTCGGCTACAGCCAG |
| NT | | CAGCAACAGGAGAAGATCAAACCGAAGGTTCGTTCGACAGTGGCGCAGCAC |
| sequence | | CACGAGGCACTGGTCGGCCACGGGTTTACACACGCGCACATCGTTGCGTTA |
| | | AGCCAACACCCGGCAGCGTTAGGGACCGTCGCTGTCAAGTATCAGGACATG |
| | | ATCGCAGCGTTGCCAGAGGCGACACACGAAGCGATCGTTGGCGTCGGCAAA |
| | | CAGTGGTCCGGCGCACGCGCTCTGGAGGCCTTGCTCACGGTGGCGGGAGAG |
| | | TTGAGAGGTCCACCGTTACAGTTGGACACAGGCCAACTTCTCAAGATTGCA |
| | | AAACGTGGCGGCGTGACCGCAGTGGAGGCAGTGCATGCATGGCGCAATGCA |
| | | CTGACGGGTGCCCCGCTCAACTTGACCCCGGAGCAGGTGGTGGCCATCGCC |
| | | AGCAATATTGGTGGCAAGCAGGCGCTGGAGACGGTGCAGGCGCTGTTGCCG |
| | | GTGCTGTGCCAGGCCCACGGCTTGACCCCGGAGCAGGTGGTGGCCATCGCC |
| | | AGCCACGATGGCGGCAAGCAGGCGCTGGAGACGGTCCAGCGGCTGTTGCCG |
| | | GTGCTGTGCCAGGCCCACGGCTTGACCCCGGAGCAGGTGGTGGCCATCGCC |
| | | AGCCACGATGGCGGCAAGCAGGCGCTGGAGACGGTCCAGCGGCTGTTGCCG |
| | | GTGCTGTGCCAGGCCCACGGCTTGACCCCCCAGCAGGTGGTGGCCATCGCC |
| | | AGCAATGGCGGTGGCAAGCAGGCGCTGGAGACGGTCCAGCGGCTGTTGCCG |
| | | GTGCTGTGCCAGGCCCACGGCTTGACCCCGGAGCAGGTGGTGGCCATCGCC |
| | | AGCCACGATGGCGGCAAGCAGGCGCTGGAGACGGTCCAGCGGCTGTTGCCG |
| | | GTGCTGTGCCAGGCCCACGGCTTGACCCCCCAGCAGGTGGTGGCCATCGCC |
| | | AGCAATGGCGGTGGCAAGCAGGCGCTGGAGACGGTCCAGCGGCTGTTGCCG |
| | | GTGCTGTGCCAGGCCCACGGCTTGACCCCCCAGCAGGTGGTGGCCATCGCC |
| | | AGCAATAATGGTGGCAAGCAGGCGCTGGAGACGGTCCAGCGGCTGTTGCCG |
| | | GTGCTGTGCCAGGCCCACGGCTTGACCCCCCAGCAGGTGGTGGCCATCGCC |
| | | AGCAATGGCGGTGGCAAGCAGGCGCTGGAGACGGTCCAGCGGCTGTTGCCG |
| | | GTGCTGTGCCAGGCCCACGGCTTGACCCCCCAGCAGGTGGTGGCCATCGCC |
| | | AGCAATAATGGTGGCAAGCAGGCGCTGGAGACGGTCCAGCGGCTGTTGCCG |
| | | GTGCTGTGCCAGGCCCACGGCTTGACCCCCCAGCAGGTGGTGGCCATCGCC |
| | | AGCAATAATGGTGGCAAGCAGGCGCTGGAGACGGTCCAGCGGCTGTTGCCG |
| | | GTGCTGTGCCAGGCCCACGGCTTGACCCCCCAGCAGGTGGTGGCCATCGCC |
| | | AGCAATAATGGTGGCAAGCAGGCGCTGGAGACGGTCCAGCGGCTGTTGCCG |
| | | GTGCTGTGCCAGGCCCACGGCTTGACCCCCCAGCAGGTGGTGGCCATCGCC |
| | | AGCAATAATGGTGGCAAGCAGGCGCTGGAGACGGTCCAGCGGCTGTTGCCG |
| | | GTGCTGTGCCAGGCCCACGGCTTGACCCCGGAGCAGGTGGTGGCCATCGCC |
| | | AGCCACGATGGCGGCAAGCAGGCGCTGGAGACGGTCCAGCGGCTGTTGCCG |
| | | GTGCTGTGCCAGGCCCACGGCTTGACCCCGGAGCAGGTGGTGGCCATCGCC |
| | | AGCCACGATGGCGGCAAGCAGGCGCTGGAGACGGTCCAGCGGCTGTTGCCG |
| | | GTGCTGTGCCAGGCCCACGGCTTGACCCCGGAGCAGGTGGTGGCCATCGCC |
| | | AGCAATATTGGTGGCAAGCAGGCGCTGGAGACGGTGCAGGCGCTGTTGCCG |
| | | GTGCTGTGCCAGGCCCACGGCTTGACCCCTCAGCAGGTGGTGGCCATCGCC |
| | | AGCAATGGCGGCGGCAGGCCGGCGCTGGAGAGCATTGTTGCCCAGTTATCT |
| | | CGCCCTGATCCGGCGTTGGCCGCGTTGACCAACGACCACCTCGTCGCCTTG |
| | | GCCTGCCTCGGCGGGCGTCCTGCGCTGGATGCAGTGAAAAAGGGATTGGGG |
| | | GATCCTATCAGCCGTTCCCAGCTGGTGAAGTCCGAGCTGGAGGAGAAGAAA |
| | | TCCGAGTTGAGGCACAAGCTGAAGTACGTGCCCCACGAGTACATCGAGCTG |
| | | ATCGAGATCGCCCGGAACAGCACCCAGGACCGTATCCTGGAGATGAAGGTG |
| | | ATGGAGTTCTTCATGAAGGTGTACGGCTACAGGGGCAAGCACCTGGGCGGC |
| | | TCCAGGAAGCCCGACGGCGCCATCTACACCGTGGGCTCCCCCATCGACTAC |
| | | GGCGTGATCGTGGACACCAAGGCCTACTCCGGCGGCTACAACCTGCCCATC |
| | | GGCCAGGCCGACGAAATGCAGAGGTACGTGGAGGAGAACCAGACCAGGAAC |
| | | AAGCACATCAACCCCAACGAGTGGTGGAAGGTGTACCCCTCCAGCGTGACC |
| | | GAGTTCAAGTTCCTGTTCGTGTCCGGCCACTTCAAGGGCAACTACAAGGCC |
| | | CAGCTGACCAGGCTGAACCACATCACCAACTGCAACGGCGCCGTGCTGTCC |
| | | GTGGAGGAGCTCCTGATCGGCGGCGAGATGATCAAGGCCGGCACCCTGACC |
| | | CTGGAGGAGGTGAGGAGGAAGTTCAACAACGGCGAGATCAACTTCGCGGCC |
| | | GACTGATAA |
| PD-1 target | SEQ ID NO: | TTCTCCCCAGCCCTGCTcgtggtgaccgaaggGGACAACGCCACCT |
| site | 18 | TCA |
| sequence | | |

### 2. TIGIT

TIGIT is a cell surface protein that is expressed on regulatory, memory and activated T cells. TIGIT belongs to the poliovirus receptor (PVR) family of immunoglobulin proteins and suppresses T-cell activation. (Yu et al., Nat Immunol., 2009, 10 (1): 48-57).

The expression of TIGIT in TILs is silenced or reduced in accordance with compositions and methods of the present invention. According to particular embodiments, expression of both PD-1 and TIGIT in TILs are silenced or reduced in accordance with compositions and methods of the present invention. For example, a method for expanding tumor infiltrating lymphocytes (TILs) into a therapeutic population of TILs may be carried out in accordance with any embodiment of the methods described herein, wherein the method comprises gene-editing at least a portion of the TILs by silencing or repressing the expression of TIGIT. As described in more detail below, the gene-editing process may comprise the use of a programmable nuclease that mediates the generation of a double-strand or single-strand break at an immune checkpoint gene, such as TIGIT. For example, a CRISPR method, a TALE method, or a zinc finger method may be used to silence or repress the expression of TIGIT in the TILs. In some embodiments, TIGIT is silenced using a TALEN knockout. In some embodiments, TIGIT is silenced using a TALE-KRAB transcriptional inhibitor knock in. More details on these methods can be found in Boettcher and McManus, Mol. Cell Review, 2015, 58, 575-585. In some embodiments, a TALEN method may be used to silence or reduce the expression of PD-1 and TIGIT in the TILs.

According to particular embodiments, expression of TIGIT in TILs is silenced or reduced in accordance with compositions and methods of the present invention, and wherein the genetically modified TILs are produced by introducing into the TILs nucleic acids, optionally mRNAs, encoding one or more TALE-nucleases able to selectively inactivate by DNA cleavage a gene encoding TIGIT, wherein the one or more TALE-nucleases comprise a TALE-nuclease that is directed against a nucleic acid sequence comprising the nucleic acid sequence of SEQ ID NO: 23 or 28, wherein the method comprises TALEN gene-editing at least a portion of the TILs by silencing or repressing the expression of TIGIT. In some embodiments, the invention provides a TALEN directed against a nucleic acid sequence comprising the nucleic acid sequence of SEQ ID NO: 23 or 28. In some embodiments, the invention provides a mRNA encoding a TALEN directed against a nucleic acid sequence comprising the nucleic acid sequence of SEQ ID NO: 23 or 28.

Examples of TALE-nucleases, and TALE-nuclease-encoding sequences, targeting the TIGIT gene are provided in the following Table 4. According to particular embodiments, TALE-nucleases according to the invention recognize and cleave a target sequence of SEQ ID NO: 23 or 28. In some embodiments, the invention provides a TALEN having the amino acid sequence of SEQ ID NO: 20, 22, 25 or 27. In some embodiments, the invention provides an mRNA sequence that encodes a TALEN having the amino acid sequence of SEQ ID NO: 20, 22, 25 or 27. In some embodiments, the invention provides a TALEN encoding nucleotide sequence of SEQ ID NO: 19, 21, 24 or 26.

TABLE 4
TIGIT KO TALE-nucleases and sequences of TALE-nuclease cleavage site in the
human TIGIT gene

| Sequence | Ref. | |
|---|---|---|
| name | Sequences | Polynucleotide or polypeptide sequences |
| Left TIGIT | SEQ ID NO: 19 | ATGGGCGATCCTAAAAAGAAACGTAAGGTCATCGATATCGCCGATC |
| target 1 TALEN | | TACGCACGCTCGGCTACAGCCAGCAGCAACAGGAGAAGATCAAACC |
| Nucleotide | | GAAGGTTCGTTCGACAGTGGCGCAGCACCACGAGGCACTGGTCGGC |
| sequence | | CACGGGTTTACACACGCGCACATCGTTGCGTTAAGCCAACACCCGG |
| | | CAGCGTTAGGGACCGTCGCTGTCAAGTATCAGGACATGATCGCAGC |
| | | GTTGCCAGAGGCGACACACGAAGCGATCGTTGGCGTCGGCAAACAG |
| | | TGGTCCGGCGCACGCGCTCTGGAGGCCTTGCTCACGGTGGCGGGAG |
| | | AGTTGAGAGGTCCACCGTTACAGTTGGACACAGGCCAACTTCTCAA |
| | | GATTGCAAAACGTGGCGGCGTGACCGCAGTGGAGGCAGTGCATGCA |
| | | TGGCGCAATGCACTGACGGGTGCCCCGCTCAACTTGACCCCCGACC |
| | | AAGTGGTTGCCATAGCGAGTAATAACGGAGGGAAACAAGCCCTCGA |
| | | AACTGTGCAAAGGCTTTTGCCTGTACTTTGCCAGGACCACGGACTC |
| | | ACGCCCGACCAGGTCGTCGCTATAGCTTCAAACGGCGGGGGCAAGC |
| | | AGGCTCTGGAAACAGTCCAACGCCTTCTGCCAGTTCTGTGTCAAGA |
| | | CCACGGGCTTACGCCGGACCAAGTAGTGGCTATCGCCTCACACGAC |
| | | GGTGGTAAGCAAGCGCTGGAAACCGTACAGCGGTTGCTTCCAGTTC |
| | | TGTGCCAAGACCACGGCTTGACCCCGGACCAAGTTGTCGCAATTGC |
| | | TTCAAATATCGGTGGTAAACAGGCCTTGGAGACGGTGCAAGCCCTT |
| | | TTGCCCGTTTTGTGTCAAGACCACGGTTTGACGCCGGACCAGGTCG |
| | | TAGCCATAGCCTCTCACGACGGTGGTAAGCAAGCACTGGAAACCGT |
| | | CCAGCGACTTCTTCCAGTACTTTGTCAAGACCATGGACTGACCCCC |
| | | GACCAAGTGGTGGCGATTGCATCTCACGACGGAGGGAAGCAAGCTC |
| | | TGGAGACTGTCCAGAGGTTGCTCCCGGTGCTCTGCCAAGACCACGG |
| | | CTTGACCCCGGACCAGGTAGTGGCGATTGCAAGTAATGGCGGGGGA |
| | | AAGCAAGCGCTCGAGACAGTTCAGCGGCTTCTCCCCGTTTTGTGTC |
| | | AAGACCACGGCCTTACGCCAGACCAAGTGGTAGCCATAGCATCTCA |
| | | CGACGGAGGTAAGCAAGCATTGGAAACTGTTCAGAGGCTCTTGCCG |
| | | GTCCTGTGTCAGGACCACGGCCTTACACCTGACCAGGTCGTTGCTA |
| | | TTGCAAGCAACGGCGGGGGGAAGCAGGCGCTGGAAACAGTGCAGCG |
| | | GTTGCTGCCCGTCCTTTGCCAAGACCACGGCTTGACTCCTGACCAG |
| | | GTGGTGGCTATAGCTTCACATGACGGGGGGAAGCAGGCTCTCGAGA |
| | | CAGTTCAGCGCCTTCTTCCTGTATTGTGCCAAGACCATGGGCTCAC |
| | | CCCAGACCAGGTTGTTGCTATAGCCAGTCACGACGGGGGTAAGCAA |
| | | GCACTTGAAACGGTGCAGAGGCTTCTCCCAGTCCTTTGTCAGGACC |
| | | ATGGGCTGACACCTGACCAGGTTGTTGCCATAGCGAGTAATGGCGG |
| | | TGGCAAGCAGGCTCTGGAGACGGTCCAAAGGCTGTTGCCCGTCCTC |
| | | TGCCAAGACCACGGCTTGACACCCGACCAAGTGGTGGCGATAGCAT |
| | | CTCACGACGGGGGCAAGCAAGCGTTGGAGACTGTTCAAAGGCTTCT |
| | | CCCTGTTCTTTGCCAAGACCACGGTTTGACTCCAGACCAAGTCGTA |
| | | GCTATAGCAAGTCACGACGGTGGTAAGCAGGCTCTCGAAACTGTTC |
| | | AGAGGCTCCTCCCAGTCCTTTGTCAAGACCATGGACTGACACCGGA |
| | | CCAGGTCGTCGCTATAGCTTCTAACATCGGGGGGAAACAAGCTTTG |
| | | GAAACAGTACAGGCCCTTCTGCCAGTTCTCTGCCAAGACCACGGTC |
| | | TTACCCCTCAGCAGGTGGTGGCCATCGCCAGCAATGGCGGCGGCAG |
| | | GCCGGCGCTGGAGAGCATTGTTGCCCAGTTATCTCGCCCTGATCCG |
| | | GCGTTGGCCGCGTTGACCAACGACCACCTCGTCGCCTTGGCCTGCC |
| | | TCGGCGGGCGTCCTGCGCTGGATGCAGTGAGAAGGGGATTGGGGGA |
| | | TCCTATCAGCCGTTCCCAGCTGGTGAAGTCCGAGCTGGAGGAGAAG |
| | | AAATCCGAGTTGAGGCACAAGCTGAAGTACGTGCCCCACGAGTACA |
| | | TCGAGCTGATCGAGATCGCCCGGAACAGCACCCAGGACCGTATCCT |
| | | GGAGATGAAGGTGATGGAGTTCTTCATGAAGGTGTACGGCTACAGG |
| | | GGCAAGCACCTGGGCGGCTCCAGGAAGCCCGACGGCGCCATCTACA |
| | | CCGTGGGCTCCCCCATCGACTACGGCGTGATCGTGGACACCAAGGC |
| | | CTACTCCGGCGGCTACAACCTGCCCATCGGCCAGGCCGACGAAATG |
| | | CAGAGGTACGTGGAGGAGAACCAGACCAGGAACAAGCACATCAACC |
| | | CCAACGAGTGGTGGAAGGTGTACCCCTCCAGCGTGACCGAGTTCAA |
| | | GTTCCTGTTCGTGTCCGGCCACTTCAAGGGCAACTACAAGGCCCAG |
| | | CTGACCAGGCTGAACCACATCACCAACTGCAACGGCGCCGTGCTGT |
| | | CCGTGGAGGAGCTCCTGATCGGCGGCGAGATGATCAAGGCCGGCAC |
| | | CCTGACCCTGGAGGAGGTGAGGAGGAAGTTCAACAACGGCGAGATC |
| | | AACTTCGCGGCCGACTGATAA |
| Left TIGIT | SEQ ID NO: 20 | MGDPKKKRKVIDIADLRTLGYSQQQQEKIKPKVRSTVAQHHEALVG |
| target 1 TALEN | | HGFTHAHIVALSQHPAALGTVAVKYQDMIAALPEATHEAIVGVGKQ |
| Amino acid | | WSGARALEALLTVAGELRGPPLQLDTGQLLKIAKRGGVTAVEAVHA |
| sequence | | WRNALTGAPLNLTPDQVVAIASNNGGKQALETVQRLLPVLCQDHGL |
| | | TPDQVVAIASNGGGKQALETVQRLLPVLCQDHGLTPDQVVAIASHD |
| | | GGKQALETVQRLLPVLCQDHGLTPDQVVAIASNIGGKQALETVQAL |
| | | LPVLCQDHGLTPDQVVAIASHDGGKQALETVORLLPVLCQDHGLTP |
| | | DQVVAIASHDGGKQALETVORLLPVLCQDHGLTPDQVVAIASNGGG |
| | | KQALETVQRLLPVLCQDHGLTPDQVVAIASHDGGKQALETVQRLLP |
| | | VLCQDHGLTPDQVVAIASNGGGKQALETVQRLLPVLCQDHGLTPDQ |
| | | VVAIASHDGGKQALETVORLLPVLCQDHGLTPDQVVAIASHDGGKQ |
| | | ALETVQRLLPVLCQDHGLTPDQVVAIASNGGGKQALETVORLLPVL |
| | | CQDHGLTPDQVVAIASHDGGKQALETVQRLLPVLCQDHGLTPDQVV |
| | | AIASHDGGKQALETVQRLLPVLCQDHGLTPDQVVAIASNIGGKQAL |
| | | ETVQALLPVLCQDHGLTPQQVVAIASNGGGRPALESIVAQLSRPDP |
| | | ALAALTNDHLVALACLGGRPALDAVRRGLGDPISRSQLVKSELEEK |
| | | KSELRHKLKYVPHEYIELIEIARNSTQDRILEMKVMEFFMKVYGYR |
| | | GKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQ |
| | | LTRLNHITNCNGAVLSVEELLIGGEMIKAGTLTLEEVRRKFNNGEI |
| | | NFAAD |
| Right TIGIT | SEQ ID NO: 21 | ATGGGCGATCCTAAAAAGAAACGTAAGGTCATCGATATCGCCGATC |
| target 1 TALEN | | GAAGGTTCGTTCGACAGTGGCGCAGCACCACGAGGCACTGGTCGGC |
| Nucleotide | | TACGCACGCTCGGCTACAGCCAGCAGCAACAGGAGAAGATCAAACC |
| sequence | | CACGGGTTTACACACGCGCACATCGTTGCGTTAAGCCAACACCCGG |
| | | CAGCGTTAGGGACCGTCGCTGTCAAGTATCAGGACATGATCGCAGC |
| | | GTTGCCAGAGGCGACACACGAAGCGATCGTTGGCGTCGGCAAACAG |
| | | TGGTCCGGCGCACGCGCTCTGGAGGCCTTGCTCACGGTGGCGGGAG |
| | | AGTTGAGAGGTCCACCGTTACAGTTGGACACAGGCCAACTTCTCAA |
| | | GATTGCAAAACGTGGCGGCGTGACCGCAGTGGAGGCAGTGCATGCA |
| | | TGGCGCAATGCACTGACGGGTGCCCCGCTCAACTTGACCCCCGACC |
| | | AAGTGGTTGCCATAGCGAGTCACGACGGAGGGAAACAAGCCCTCGA |
| | | AACTGTGCAAAGGCTTTTGCCTGTACTTTGCCAGGACCACGGACTC |
| | | ACGCCCGACCAGGTCGTCGCTATAGCTTCACATGACGGGGGCAAGC |
| | | AGGCTCTGGAAACAGTCCAACGCCTTCTGCCAGTTCTGTGTCAAGA |
| | | CCACGGGCTTACGCCGGACCAAGTAGTGGCTATCGCCTCACACGAC |
| | | GGTGGTAAGCAAGCGCTGGAAACCGTACAGCGGTTGCTTCCAGTTC |
| | | TGTGCCAAGACCACGGCTTGACCCCGGACCAAGTTGTCGCAATTGC |
| | | TTCAAATATCGGTGGTAAACAGGCCTTGGAGACGGTGCAAGCCCTT |
| | | TTGCCCGTTTTGTGTCAAGACCACGGTTTGACGCCGGACCAGGTCG |
| | | TAGCCATAGCCTCTAACAATGGTGGTAAGCAAGCACTGGAAACCGT |
| | | CCAGCGACTTCTTCCAGTACTTTGTCAAGACCATGGACTGACCCCC |
| | | GACCAAGTGGTGGCGATTGCATCTAATGGCGGAGGGAAGCAAGCTC |
| | | TGGAGACTGTCCAGAGGTTGCTCCCGGTGCTCTGCCAAGACCACGG |
| | | CTTGACCCCGGACCAGGTAGTGGCGATTGCAAGTAATGGCGGGGGA |
| | | AAGCAAGCGCTCGAGACAGTTCAGCGGCTTCTCCCCGTTTTGTGTC |
| | | AAGACCACGGCCTTACGCCAGACCAAGTGGTAGCCATAGCATCTAA |
| | | CAACGGAGGTAAGCAAGCATTGGAAACTGTTCAGAGGCTCTTGCCG |
| | | GTCCTGTGTCAGGACCACGGCCTTACACCTGACCAGGTCGTTGCTA |
| | | TTGCAAGCAACATCGGGGGGAAGCAGGCGCTGGAAACAGTGCAGGC |
| | | CTTGCTGCCCGTCCTTTGCCAAGACCACGGCTTGACTCCTGACCAG |
| | | GTGGTGGCTATAGCTTCACATGACGGGGGGAAGCAGGCTCTCGAGA |
| | | CAGTTCAGCGCCTTCTTCCTGTATTGTGCCAAGACCATGGGCTCAC |
| | | CCCAGACCAGGTTGTTGCTATAGCCAGTCACGACGGGGGTAAGCAA |
| | | GCACTTGAAACGGTGCAGAGGCTTCTCCCAGTCCTTTGTCAGGACC |
| | | ATGGGCTGACACCTGACCAGGTTGTTGCCATAGCGAGTAATGGCGG |
| | | TGGCAAGCAGGCTCTGGAGACGGTCCAAAGGCTGTTGCCCGTCCTC |
| | | TGCCAAGACCACGGCTTGACACCCGACCAAGTGGTGGCGATAGCAT |
| | | CTAACAACGGGGGCAAGCAAGCGTTGGAGACTGTTCAAAGGCTTCT |
| | | CCCTGTTCTTTGCCAAGACCACGGTTTGACTCCAGACCAAGTCGTA |
| | | GCTATAGCAAGTAATAACGGTGGTAAGCAGGCTCTCGAAACTGTTC |
| | | AGAGGCTCCTCCCAGTCCTTTGTCAAGACCATGGACTGACACCGGA |
| | | CCAGGTCGTCGCTATAGCTTCTAACAACGGGGGGAAACAAGCTTTG |
| | | GAAACAGTACAGCGGCTTCTGCCAGTTCTCTGCCAAGACCACGGTC |
| | | TTACCCCTCAGCAGGTGGTGGCCATCGCCAGCAATGGCGGCGGCAG |
| | | GCCGGCGCTGGAGAGCATTGTTGCCCAGTTATCTCGCCCTGATCCG |
| | | GCGTTGGCCGCGTTGACCAACGACCACCTCGTCGCCTTGGCCTGCC |
| | | TCGGCGGGCGTCCTGCGCTGGATGCAGTGAGAAGGGGATTGGGGGA |
| | | TCCTATCAGCCGTTCCCAGCTGGTGAAGTCCGAGCTGGAGGAGAAG |
| | | AAATCCGAGTTGAGGCACAAGCTGAAGTACGTGCCCCACGAGTACA |
| | | TCGAGCTGATCGAGATCGCCCGGAACAGCACCCAGGACCGTATCCT |
| | | GGAGATGAAGGTGATGGAGTTCTTCATGAAGGTGTACGGCTACAGG |
| | | GGCAAGCACCTGGGCGGCTCCAGGAAGCCCGACGGCGCCATCTACA |
| | | CCGTGGGCTCCCCCATCGACTACGGCGTGATCGTGGACACCAAGGC |
| | | CTACTCCGGCGGCTACAACCTGCCCATCGGCCAGGCCGACGAAATG |
| | | CAGAGGTACGTGGAGGAGAACCAGACCAGGAACAAGCACATCAACC |
| | | CCAACGAGTGGTGGAAGGTGTACCCCTCCAGCGTGACCGAGTTCAA |
| | | GTTCCTGTTCGTGTCCGGCCACTTCAAGGGCAACTACAAGGCCCAG |
| | | CTGACCAGGCTGAACCACATCACCAACTGCAACGGCGCCGTGCTGT |
| | | CCGTGGAGGAGCTCCTGATCGGCGGCGAGATGATCAAGGCCGGCAC |
| | | CCTGACCCTGGAGGAGGTGAGGAGGAAGTTCAACAACGGCGAGATC |
| | | AACTTCGCGGCCGACTGATAA |
| Right TIGIT | SEQ ID NO: 22 | MGDPKKKRKVIDIADLRTLGYSQQQQEKIKPKVRSTVAQHHEALVG |
| target 1 TALEN | | HGFTHAHIVALSQHPAALGTVAVKYQDMIAALPEATHEAIVGVGKO |
| Amino acid | | WSGARALEALLTVAGELRGPPLOLDTGOLLKIAKRGGVTAVEAVHA |
| sequence | | WRNALTGAPLNLTPDQVVAIASHDGGKQALETVORLLPVLCQDHGL |
| | | TPDQVVAIASHDGGKQALETVQRLLPVLCQDHGLTPDQVVAIASHD |
| | | GGKQALETVQRLLPVLCQDHGLTPDQVVAIASNIGGKQALETVQAL |
| | | LPVLCQDHGLTPDQVVAIASNNGGKQALETVQRLLPVLCQDHGLTP |
| | | DQVVAIASNGGGKQALETVQRLLPVLCQDHGLTPDQVVAIASNGGG |
| | | KQALETVQRLLPVLCQDHGLTPDQVVAIASNNGGKQALETVORLLP |
| | | VLCQDHGLTPDQVVAIASNIGGKQALETVQALLPVLCQDHGLTPDQ |
| | | VVAIASHDGGKQALETVQRLLPVLCQDHGLTPDQVVAIASHDGGKQ |
| | | ALETVQRLLPVLCQDHGLTPDQVVAIASNGGGKQALETVQRLLPVL |
| | | CQDHGLTPDQVVAIASNNGGKQALETVQRLLPVLCQDHGLTPDQVV |
| | | AIASNNGGKQALETVQRLLPVLCQDHGLTPDQVVAIASNNGGKQAL |
| | | ETVQRLLPVLCQDHGLTPQQVVAIASNGGGRPALESIVAQLSRPDP |
| | | ALAALTNDHLVALACLGGRPALDAVRRGLGDPISRSQLVKSELEEK |
| | | KSELRHKLKYVPHEYIELIEIARNSTQDRILEMKVMEFFMKVYGYR |
| | | GKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQ |
| | | LTRLNHITNCNGAVLSVEELLIGGEMIKAGTLTLEEVRRKFNNGEI |
| | | NFAAD |
| TIGIT target | SEQ ID NO: 23 | TGTCACCTCTCCTCCACcacggcacaagtgACCCAGGTCAACTGGG |
| site sequence 1 | | A |
| Left TIGIT | SEQ ID NO: 24 | ATGGGCGATCCTAAAAAGAAACGTAAGGTCATCGATATCGCCGATC |
| target 2 TALEN | | TACGCACGCTCGGCTACAGCCAGCAGCAACAGGAGAAGATCAAACC |
| Nucleotide | | GAAGGTTCGTTCGACAGTGGCGCAGCACCACGAGGCACTGGTCGGC |
| sequence | | CACGGGTTTACACACGCGCACATCGTTGCGTTAAGCCAACACCCGG |
| | | CAGCGTTAGGGACCGTCGCTGTCAAGTATCAGGACATGATCGCAGC |
| | | GTTGCCAGAGGCGACACACGAAGCGATCGTTGGCGTCGGCAAACAG |
| | | TGGTCCGGCGCACGCGCTCTGGAGGCCTTGCTCACGGTGGCGGGAG |
| | | AGTTGAGAGGTCCACCGTTACAGTTGGACACAGGCCAACTTCTCAA |
| | | GATTGCAAAACGTGGCGGCGTGACCGCAGTGGAGGCAGTGCATGCA |
| | | TGGCGCAATGCACTGACGGGTGCCCCGCTCAACTTGACCCCCGACC |
| | | AAGTGGTTGCCATAGCGAGTAATGGTGGAGGGAAACAAGCCCTCGA |
| | | AACTGTGCAAAGGCTTTTGCCTGTACTTTGCCAGGACCACGGACTC |
| | | ACGCCCGACCAGGTCGTCGCTATAGCTTCAAACAACGGGGGCAAGC |
| | | AGGCTCTGGAAACAGTCCAACGCCTTCTGCCAGTTCTGTGTCAAGA |
| | | CCACGGGCTTACGCCGGACCAAGTAGTGGCTATCGCCTCAAACGGC |
| | | GGTGGTAAGCAAGCGCTGGAAACCGTACAGCGGTTGCTTCCAGTTC |
| | | TGTGCCAAGACCACGGCTTGACCCCGGACCAAGTTGTCGCAATTGC |
| | | TTCACACGACGGTGGTAAACAGGCCTTGGAGACGGTGCAAAGGCTT |
| | | TTGCCCGTTTTGTGTCAAGACCACGGTTTGACGCCGGACCAGGTCG |
| | | TAGCCATAGCCTCTCACGACGGTGGTAAGCAAGCACTGGAAACCGT |
| | | CCAGCGACTTCTTCCAGTACTTTGTCAAGACCATGGACTGACCCCC |
| | | GACCAAGTGGTGGCGATTGCATCTAATGGCGGAGGGAAGCAAGCTC |
| | | TGGAGACTGTCCAGAGGTTGCTCCCGGTGCTCTGCCAAGACCACGG |
| | | CTTGACCCCGGACCAGGTAGTGGCGATTGCAAGTCACGACGGGGGA |
| | | AAGCAAGCGCTCGAGACAGTTCAGCGGCTTCTCCCCGTTTTGTGTC |
| | | AAGACCACGGCCTTACGCCAGACCAAGTGGTAGCCATAGCATCTCA |
| | | CGACGGAGGTAAGCAAGCATTGGAAACTGTTCAGAGGCTCTTGCCG |
| | | GTCCTGTGTCAGGACCACGGCCTTACACCTGACCAGGTCGTTGCTA |
| | | TTGCAAGCCACGACGGGGGGAAGCAGGCGCTGGAAACAGTGCAGCG |
| | | GTTGCTGCCCGTCCTTTGCCAAGACCACGGCTTGACTCCTGACCAG |
| | | GTGGTGGCTATAGCTTCAAACGGCGGGGGGAAGCAGGCTCTCGAGA |
| | | CAGTTCAGCGCCTTCTTCCTGTATTGTGCCAAGACCATGGGCTCAC |
| | | CCCAGACCAGGTTGTTGCTATAGCCAGTCACGACGGGGGTAAGCAA |
| | | GCACTTGAAACGGTGCAGAGGCTTCTCCCAGTCCTTTGTCAGGACC |
| | | ATGGGCTGACACCTGACCAGGTTGTTGCCATAGCGAGTAATGGCGG |
| | | TGGCAAGCAGGCTCTGGAGACGGTCCAAAGGCTGTTGCCCGTCCTC |
| | | TGCCAAGACCACGGCTTGACACCCGACCAAGTGGTGGCGATAGCAT |
| | | CTAACATCGGGGGCAAGCAAGCGTTGGAGACTGTTCAAGCCCTTCT |
| | | CCCTGTTCTTTGCCAAGACCACGGTTTGACTCCAGACCAAGTCGTA |
| | | GCTATAGCAAGTAATAACGGTGGTAAGCAGGCTCTCGAAACTGTTC |
| | | AGAGGCTCCTCCCAGTCCTTTGTCAAGACCATGGACTGACACCGGA |
| | | CCAGGTCGTCGCTATAGCTTCTAACGGCGGGGGGAAACAAGCTTTG |
| | | GAAACAGTACAGCGGCTTCTGCCAGTTCTCTGCCAAGACCACGGTC |
| | | TTACCCCTCAGCAGGTGGTGGCCATCGCCAGCAATGGCGGCGGCAG |
| | | GCCGGCGCTGGAGAGCATTGTTGCCCAGTTATCTCGCCCTGATCCG |
| | | GCGTTGGCCGCGTTGACCAACGACCACCTCGTCGCCTTGGCCTGCC |
| | | TCGGCGGGCGTCCTGCGCTGGATGCAGTGAGAAGGGGATTGGGGGA |
| | | TCCTATCAGCCGTTCCCAGCTGGTGAAGTCCGAGCTGGAGGAGAAG |
| | | AAATCCGAGTTGAGGCACAAGCTGAAGTACGTGCCCCACGAGTACA |
| | | TCGAGCTGATCGAGATCGCCCGGAACAGCACCCAGGACCGTATCCT |
| | | GGAGATGAAGGTGATGGAGTTCTTCATGAAGGTGTACGGCTACAGG |
| | | GGCAAGCACCTGGGCGGCTCCAGGAAGCCCGACGGCGCCATCTACA |
| | | CCGTGGGCTCCCCCATCGACTACGGCGTGATCGTGGACACCAAGGC |
| | | CTACTCCGGCGGCTACAACCTGCCCATCGGCCAGGCCGACGAAATG |
| | | CAGAGGTACGTGGAGGAGAACCAGACCAGGAACAAGCACATCAACC |
| | | CCAACGAGTGGTGGAAGGTGTACCCCTCCAGCGTGACCGAGTTCAA |
| | | GTTCCTGTTCGTGTCCGGCCACTTCAAGGGCAACTACAAGGCCCAG |
| | | CTGACCAGGCTGAACCACATCACCAACTGCAACGGCGCCGTGCTGT |
| | | CCGTGGAGGAGCTCCTGATCGGCGGCGAGATGATCAAGGCCGGCAC |
| | | CCTGACCCTGGAGGAGGTGAGGAGGAAGTTCAACAACGGCGAGATC |
| | | AACTTCGCGGCCGACTGATAA |
| | | MGDPKKKRKVIDIADLRTLGYSQQQQEKIKPKVRSTVAQHHEALVG |
| | | HGFTHAHIVALSQHPAALGTVAVKYQDMIAALPEATHEAIVGVGKQ |
| | | WSGARALEALLTVAGELRGPPLQLDTGQLLKIAKRGGVTAVEAVHA |
| | | WRNALTGAPLNLTPDQVVAIASNGGGKQALETVORLLPVLCQDHGL |
| Left TIGIT | SEQ ID NO: 25 | TPDQVVAIASNNGGKQALETVORLLPVLCQDHGLTPDQVVAIASNG |
| target 2 TALEN | | GGKQALETVQRLLPVLCQDHGLTPDQVVAIASHDGGKQALETVQRL |
| Amino acid | | LPVLCQDHGLTPDQVVAIASHDGGKQALETVQRLLPVLCQDHGLTP |
| sequence | | DQVVAIASNGGGKQALETVQRLLPVLCQDHGLTPDQVVAIASHDGG |
| | | KQALETVQRLLPVLCQDHGLTPDQVVAIASHDGGKQALETVORLLP |
| | | VLCQDHGLTPDQVVAIASHDGGKQALETVQRLLPVLCQDHGLTPDQ |
| | | VVAIASNGGGKQALETVQRLLPVLCQDHGLTPDQVVAIASHDGGKQ |
| | | ALETVQRLLPVLCQDHGLTPDQVVAIASNGGGKQALETVORLLPVL |
| | | CQDHGLTPDQVVAIASNIGGKQALETVQALLPVLCQDHGLTPDQVV |
| | | AIASNNGGKQALETVQRLLPVLCQDHGLTPDQVVAIASNGGGKQAL |
| | | ETVQRLLPVLCQDHGLTPQQVVAIASNGGGRPALESIVAQLSRPDP |
| | | ALAALTNDHLVALACLGGRPALDAVRRGLGDPISRSQLVKSELEEK |
| | | KSELRHKLKYVPHEYIELIEIARNSTQDRILEMKVMEFFMKVYGYR |
| | | GKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQ |
| | | LTRLNHITNCNGAVLSVEELLIGGEMIKAGTLTLEEVRRKFNNGEI |
| | | NFAAD |
| Right TIGIT | SEQ ID NO: 26 | ATGGGCGATCCTAAAAAGAAACGTAAGGTCATCGATATCGCCGATC |
| target 2 TALEN | | TACGCACGCTCGGCTACAGCCAGCAGCAACAGGAGAAGATCAAACC |
| Nucleotide | | GAAGGTTCGTTCGACAGTGGCGCAGCACCACGAGGCACTGGTCGGC |
| sequence | | CACGGGTTTACACACGCGCACATCGTTGCGTTAAGCCAACACCCGG |
| | | CAGCGTTAGGGACCGTCGCTGTCAAGTATCAGGACATGATCGCAGC |
| | | GTTGCCAGAGGCGACACACGAAGCGATCGTTGGCGTCGGCAAACAG |
| | | TGGTCCGGCGCACGCGCTCTGGAGGCCTTGCTCACGGTGGCGGGAG |
| | | AGTTGAGAGGTCCACCGTTACAGTTGGACACAGGCCAACTTCTCAA |
| | | GATTGCAAAACGTGGCGGCGTGACCGCAGTGGAGGCAGTGCATGCA |
| | | TGGCGCAATGCACTGACGGGTGCCCCGCTCAACTTGACCCCCGACC |
| | | AAGTGGTTGCCATAGCGAGTAATAACGGAGGGAAACAAGCCCTCGA |
| | | AACTGTGCAAAGGCTTTTGCCTGTACTTTGCCAGGACCACGGACTC |
| | | ACGCCCGACCAGGTCGTCGCTATAGCTTCAAACAACGGGGGCAAGC |
| | | AGGCTCTGGAAACAGTCCAACGCCTTCTGCCAGTTCTGTGTCAAGA |
| | | CCACGGGCTTACGCCGGACCAAGTAGTGGCTATCGCCTCAAACATC |
| | | GGTGGTAAGCAAGCGCTGGAAACCGTACAGGCCTTGCTTCCAGTTC |
| | | TGTGCCAAGACCACGGCTTGACCCCGGACCAAGTTGTCGCAATTGC |
| | | TTCAAATATCGGTGGTAAACAGGCCTTGGAGACGGTGCAAGCCCTT |
| | | TTGCCCGTTTTGTGTCAAGACCACGGTTTGACGCCGGACCAGGTCG |
| | | TAGCCATAGCCTCTAACGGCGGTGGTAAGCAAGCACTGGAAACCGT |
| | | CCAGCGACTTCTTCCAGTACTTTGTCAAGACCATGGACTGACCCCC |
| | | GACCAAGTGGTGGCGATTGCATCTCACGACGGAGGGAAGCAAGCTC |
| | | TGGAGACTGTCCAGAGGTTGCTCCCGGTGCTCTGCCAAGACCACGG |
| | | CTTGACCCCGGACCAGGTAGTGGCGATTGCAAGTAATGGCGGGGGA |
| | | AAGCAAGCGCTCGAGACAGTTCAGCGGCTTCTCCCCGTTTTGTGTC |
| | | AAGACCACGGCCTTACGCCAGACCAAGTGGTAGCCATAGCATCTAA |
| | | CAACGGAGGTAAGCAAGCATTGGAAACTGTTCAGAGGCTCTTGCCG |
| | | GTCCTGTGTCAGGACCACGGCCTTACACCTGACCAGGTCGTTGCTA |
| | | TTGCAAGCAACAACGGGGGGAAGCAGGCGCTGGAAACAGTGCAGCG |
| | | GTTGCTGCCCGTCCTTTGCCAAGACCACGGCTTGACTCCTGACCAG |
| | | GTGGTGGCTATAGCTTCAAACATCGGGGGGAAGCAGGCTCTCGAGA |
| | | CAGTTCAGGCCCTTCTTCCTGTATTGTGCCAAGACCATGGGCTCAC |
| | | CCCAGACCAGGTTGTTGCTATAGCCAGTAACATCGGGGGTAAGCAA |
| | | GCACTTGAAACGGTGCAGGCACTTCTCCCAGTCCTTTGTCAGGACC |
| | | ATGGGCTGACACCTGACCAGGTTGTTGCCATAGCGAGTCACGACGG |
| | | TGGCAAGCAGGCTCTGGAGACGGTCCAAAGGCTGTTGCCCGTCCTC |
| | | TGCCAAGACCACGGCTTGACACCCGACCAAGTGGTGGCGATAGCAT |
| | | CTCACGACGGGGGCAAGCAAGCGTTGGAGACTGTTCAAAGGCTTCT |
| | | CCCTGTTCTTTGCCAAGACCACGGTTTGACTCCAGACCAAGTCGTA |
| | | GCTATAGCAAGTAATGGCGGTGGTAAGCAGGCTCTCGAAACTGTTC |
| | | AGAGGCTCCTCCCAGTCCTTTGTCAAGACCATGGACTGACACCGGA |
| | | CCAGGTCGTCGCTATAGCTTCTAACAACGGGGGGAAACAAGCTTTG |
| | | GAAACAGTACAGCGGCTTCTGCCAGTTCTCTGCCAAGACCACGGTC |
| | | TTACCCCTCAGCAGGTGGTGGCCATCGCCAGCAATGGCGGCGGCAG |
| | | GCCGGCGCTGGAGAGCATTGTTGCCCAGTTATCTCGCCCTGATCCG |
| | | GCGTTGGCCGCGTTGACCAACGACCACCTCGTCGCCTTGGCCTGCC |
| | | TCGGCGGGCGTCCTGCGCTGGATGCAGTGAGAAGGGGATTGGGGGA |
| | | TCCTATCAGCCGTTCCCAGCTGGTGAAGTCCGAGCTGGAGGAGAAG |
| | | AAATCCGAGTTGAGGCACAAGCTGAAGTACGTGCCCCACGAGTACA |
| | | TCGAGCTGATCGAGATCGCCCGGAACAGCACCCAGGACCGTATCCT |
| | | GGAGATGAAGGTGATGGAGTTCTTCATGAAGGTGTACGGCTACAGG |
| | | GGCAAGCACCTGGGCGGCTCCAGGAAGCCCGACGGCGCCATCTACA |
| | | CCGTGGGCTCCCCCATCGACTACGGCGTGATCGTGGACACCAAGGC |
| | | CTACTCCGGCGGCTACAACCTGCCCATCGGCCAGGCCGACGAAATG |
| | | CAGAGGTACGTGGAGGAGAACCAGACCAGGAACAAGCACATCAACC |
| | | CCAACGAGTGGTGGAAGGTGTACCCCTCCAGCGTGACCGAGTTCAA |
| | | GTTCCTGTTCGTGTCCGGCCACTTCAAGGGCAACTACAAGGCCCAG |
| | | CTGACCAGGCTGAACCACATCACCAACTGCAACGGCGCCGTGCTGT |
| | | CCGTGGAGGAGCTCCTGATCGGCGGCGAGATGATCAAGGCCGGCAC |
| | | CCTGACCCTGGAGGAGGTGAGGAGGAAGTTCAACAACGGCGAGATC |
| | | AACTTCGCGGCCGACTGATAA |
| Right TIGIT | SEQ ID NO: 27 | MGDPKKKRKVIDIADLRTLGYSQQQQEKIKPKVRSTVAQHHEALVG |
| target 2 TALEN | | HGFTHAHIVALSQHPAALGTVAVKYQDMIAALPEATHEAIVGVGKQ |
| Amino acid | | WSGARALEALLTVAGELRGPPLQLDTGQLLKIAKRGGVTAVEAVHA |
| sequence | | WRNALTGAPLNLTPDQVVAIASNNGGKQALETVQRLLPVLCQDHGL |
| | | TPDQVVAIASNNGGKQALETVQRLLPVLCQDHGLTPDQVVAIASNI |
| | | GGKQALETVQALLPVLCQDHGLTPDQVVAIASNIGGKQALETVQAL |
| | | LPVLCQDHGLTPDQVVAIASNGGGKQALETVQRLLPVLCQDHGLTP |
| | | DOVVAIASHDGGKQALETVQRLLPVLCQDHGLTPDQVVAIASNGGG |
| | | KQALETVQRLLPVLCQDHGLTPDQVVAIASNNGGKQALETVORLLP |
| | | VLCQDHGLTPDQVVAIASNNGGKQALETVORLLPVLCQDHGLTPDQ |
| | | VVAIASNIGGKQALETVQALLPVLCQDHGLTPDQVVAIASNIGGKQ |
| | | ALETVQALLPVLCQDHGLTPDQVVAIASHDGGKQALETVORLLPVL |
| | | CQDHGLTPDQVVAIASHDGGKQALETVORLLPVLCQDHGLTPDQVV |
| | | AIASNGGGKQALETVQRLLPVLCQDHGLTPDQVVAIASNNGGKQAL |
| | | ETVQRLLPVLCQDHGLTPQQVVAIASNGGGRPALESIVAQLSRPDP |
| | | ALAALTNDHLVALACLGGRPALDAVRRGLGDPISRSQLVKSELEEK |
| | | KSELRHKLKYVPHEYIELIEIARNSTQDRILEMKVMEFFMKVYGYR |
| | | GKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQ |
| | | LTRLNHITNCNGAVLSVEELLIGGEMIKAGTLTLEEVRRKFNNGEI |
| | | NFAAD |
| TIGIT target | SEQ ID NO: 28 | TTGTCCTCCCTCTAGTGGCTGAGCACGGTGCCAGGTTCCAGATTCC |
| site sequence 2 | | A |

### 3. LAG3

Lymphocyte-activation gene 3, also known as LAG-3, is a protein which in humans is encoded by the LAG3 gene. LAG-3 is a cell surface molecule with diverse biologic effects on T cell function. It is an immune checkpoint receptor.

The expression of LAG3 in TILs is silenced or reduced in accordance with compositions and methods of the present invention. According to particular embodiments, expression of both PD-1 and LAG3 in TILs are silenced or reduced in accordance with compositions and methods of the present invention. For example, a method for expanding tumor infiltrating lymphocytes (TILs) into a therapeutic population of TILs may be carried out in accordance with any embodiment of the methods described herein, wherein the method comprises gene-editing at least a portion of the TILs by silencing or repressing the expression of LAG3. As described in more detail below, the gene-editing process may comprise the use of a programmable nuclease that mediates the generation of a double-strand or single-strand break at an immune checkpoint gene, such as LAG3. For example, a CRISPR method, a TALE method, or a zinc finger method may be used to silence or repress the expression of LAG-3 in the TILs. In some embodiments, LAG3 is silenced using a TALEN knockout. In some embodiments, LAG3 is silenced using a TALE-KRAB transcriptional inhibitor knock in. More details on these methods can be found in Boettcher and McManus, Mol. Cell Review, 2015, 58, 575-585. In some embodiments, a TALEN method may be used to silence or reduce the expression of PD-1 and LAG3 in the TILs.

According to particular embodiments, expression of LAG3 in TILs is silenced or reduced in accordance with compositions and methods of the present invention, and wherein the genetically modified TILs are produced by introducing into the TILs nucleic acids, optionally mRNAs, encoding one or more TALE-nucleases able to selectively inactivate by DNA cleavage a gene encoding TIGIT, wherein the one or more TALE-nucleases comprise a TALE-nuclease that is directed against a nucleic acid sequence comprising the nucleic acid sequence of SEQ ID NO: 36, wherein the method comprises TALEN gene-editing at least a portion of the TILs by silencing or repressing the expression of LAG3. In some embodiments, the invention provides a TALEN directed against a nucleic acid sequence comprising the nucleic acid sequence of SEQ ID NO: 36. In some embodiments, the invention provides a mRNA encoding a TALEN directed against a nucleic acid sequence comprising the nucleic acid sequence of SEQ ID NO: 36.

Examples of TALE-nucleases, and TALE-nuclease-encoding sequences, targeting the LAG3 gene are provided in the following Table 21. According to particular embodiments, TALE-nucleases according to the invention recognize and cleave a target sequence of SEQ ID NO: 36. In some embodiments, the invention provides a TALEN having the amino acid sequence of SEQ ID NO: 33 or 35. In some embodiments, the invention provides an mRNA sequence that encodes a TALEN having the amino acid sequence of SEQ ID NO: 33 or 35. In some embodiments, the invention provides a TALEN encoding nucleotide sequence of SEQ ID NO: 32 or 34.

TABLE 21
LAG3 KO TALE-nucleases and sequences of TALE-nuclease cleavage site in the
human LAG3 gene

| Sequence | | |
|---|---|---|
| name | Ref. Sequences | Polynucleotide or polypeptide sequences |
| Left LAG3 | SEQ ID NO: 32 | ATGGGCGATCCTAAAAAGAAACGTAAGGTCATCGATATCGCCGATC |
| TALEN | | TACGCACGCTCGGCTACAGCCAGCAGCAACAGGAGAAGATCAAACC |
| Nucleotide | | GAAGGTTCGTTCGACAGTGGCGCAGCACCACGAGGCACTGGTCGGC |
| sequence | | CACGGGTTTACACACGCGCACATCGTTGCGTTAAGCCAACACCCGG |
| | | CAGCGTTAGGGACCGTCGCTGTCAAGTATCAGGACATGATCGCAGC |
| | | GTTGCCAGAGGCGACACACGAAGCGATCGTTGGCGTCGGCAAACAG |
| | | TGGTCCGGCGCACGCGCTCTGGAGGCCTTGCTCACGGTGGCGGGAG |
| | | AGTTGAGAGGTCCACCGTTACAGTTGGACACAGGCCAACTTCTCAA |
| | | GATTGCAAAACGTGGCGGCGTGACCGCAGTGGAGGCAGTGCATGCA |
| | | TGGCGCAATGCACTGACGGGTGCCCCGCTCAACTTGACCCCGGAGC |
| | | AGGTGGTGGCCATCGCCAGCCACGATGGCGGCAAGCAGGCGCTGGA |
| | | GACGGTCCAGCGGCTGTTGCCGGTGCTGTGCCAGGCCCACGGCTTG |
| | | ACCCCGGAGCAGGTGGTGGCCATCGCCAGCCACGATGGCGGCAAGC |
| | | AGGCGCTGGAGACGGTCCAGCGGCTGTTGCCGGTGCTGTGCCAGGC |
| | | CCACGGCTTGACCCCGGAGCAGGTGGTGGCCATCGCCAGCAATATT |
| | | GGTGGCAAGCAGGCGCTGGAGACGGTGCAGGCGCTGTTGCCGGTGC |
| | | TGTGCCAGGCCCACGGCTTGACCCCCCAGCAGGTGGTGGCCATCGC |
| | | CAGCAATAATGGTGGCAAGCAGGCGCTGGAGACGGTCCAGCGGCTG |
| | | TTGCCGGTGCTGTGCCAGGCCCACGGCTTGACCCCCCAGCAGGTGG |
| | | TGGCCATCGCCAGCAATAATGGTGGCAAGCAGGCGCTGGAGACGGT |
| | | CCAGCGGCTGTTGCCGGTGCTGTGCCAGGCCCACGGCTTGACCCCG |
| | | GAGCAGGTGGTGGCCATCGCCAGCAATATTGGTGGCAAGCAGGCGC |
| | | TGGAGACGGTGCAGGCGCTGTTGCCGGTGCTGTGCCAGGCCCACGG |
| | | CTTGACCCCCCAGCAGGTGGTGGCCATCGCCAGCAATGGCGGTGGC |
| | | AAGCAGGCGCTGGAGACGGTCCAGCGGCTGTTGCCGGTGCTGTGCC |
| | | AGGCCCACGGCTTGACCCCGGAGCAGGTGGTGGCCATCGCCAGCCA |
| | | CGATGGCGGCAAGCAGGCGCTGGAGACGGTCCAGCGGCTGTTGCCG |
| | | GTGCTGTGCCAGGCCCACGGCTTGACCCCCCAGCAGGTGGTGGCCA |
| | | TCGCCAGCAATGGCGGTGGCAAGCAGGCGCTGGAGACGGTCCAGCG |
| | | GCTGTTGCCGGTGCTGTGCCAGGCCCACGGCTTGACCCCGGAGCAG |
| | | GTGGTGGCCATCGCCAGCCACGATGGCGGCAAGCAGGCGCTGGAGA |
| | | CGGTCCAGCGGCTGTTGCCGGTGCTGTGCCAGGCCCACGGCTTGAC |
| | | CCCGGAGCAGGTGGTGGCCATCGCCAGCAATATTGGTGGCAAGCAG |
| | | GCGCTGGAGACGGTGCAGGCGCTGTTGCCGGTGCTGTGCCAGGCCC |
| | | ACGGCTTGACCCCCCAGCAGGTGGTGGCCATCGCCAGCAATAATGG |
| | | TGGCAAGCAGGCGCTGGAGACGGTCCAGCGGCTGTTGCCGGTGCTG |
| | | TGCCAGGCCCACGGCTTGACCCCGGAGCAGGTGGTGGCCATCGCCA |
| | | GCCACGATGGCGGCAAGCAGGCGCTGGAGACGGTCCAGCGGCTGTT |
| | | GCCGGTGCTGTGCCAGGCCCACGGCTTGACCCCGGAGCAGGTGGTG |
| | | GCCATCGCCAGCCACGATGGCGGCAAGCAGGCGCTGGAGACGGTCC |
| | | AGCGGCTGTTGCCGGTGCTGTGCCAGGCCCACGGCTTGACCCCCCA |
| | | GCAGGTGGTGGCCATCGCCAGCAATGGCGGTGGCAAGCAGGCGCTG |
| | | GAGACGGTCCAGCGGCTGTTGCCGGTGCTGTGCCAGGCCCACGGCT |
| | | TGACCCCTCAGCAGGTGGTGGCCATCGCCAGCAATGGCGGCGGCAG |
| | | GCCGGCGCTGGAGAGCATTGTTGCCCAGTTATCTCGCCCTGATCCG |
| | | GCGTTGGCCGCGTTGACCAACGACCACCTCGTCGCCTTGGCCTGCC |
| | | TCGGCGGGCGTCCTGCGCTGGATGCAGTGAAAAAGGGATTGGGGGA |
| | | TCCTATCAGCCGTTCCCAGCTGGTGAAGTCCGAGCTGGAGGAGAAG |
| | | AAATCCGAGTTGAGGCACAAGCTGAAGTACGTGCCCCACGAGTACA |
| | | TCGAGCTGATCGAGATCGCCCGGAACAGCACCCAGGACCGTATCCT |
| | | GGAGATGAAGGTGATGGAGTTCTTCATGAAGGTGTACGGCTACAGG |
| | | GGCAAGCACCTGGGCGGCTCCAGGAAGCCCGACGGCGCCATCTACA |
| | | CCGTGGGCTCCCCCATCGACTACGGCGTGATCGTGGACACCAAGGC |
| | | CTACTCCGGCGGCTACAACCTGCCCATCGGCCAGGCCGACGAAATG |
| | | CAGAGGTACGTGGAGGAGAACCAGACCAGGAACAAGCACATCAACC |
| | | CCAACGAGTGGTGGAAGGTGTACCCCTCCAGCGTGACCGAGTTCAA |
| | | GTTCCTGTTCGTGTCCGGCCACTTCAAGGGCAACTACAAGGCCCAG |
| | | CTGACCAGGCTGAACCACATCACCAACTGCAACGGCGCCGTGCTGT |
| | | CCGTGGAGGAGCTCCTGATCGGCGGCGAGATGATCAAGGCCGGCAC |
| | | CCTGACCCTGGAGGAGGTGAGGAGGAAGTTCAACAACGGCGAGATC |
| | | AACTTCGCGGCCGACTGATAA |
| Left LAG3 | SEQ ID NO: 33 | MGDPKKKRKVIDIADLRTLGYSQQQQEKIKPKVRSTVAQHHEALVG |
| TALEN | | HGFTHAHIVALSQHPAALGTVAVKYQDMIAALPEATHEAIVGVGKQ |
| Amino acid | | WSGARALEALLTVAGELRGPPLQLDTGOLLKIAKRGGVTAVEAVHA |
| sequence | | WRNALTGAPLNLTPEQVVAIASHDGGKQALETVORLLPVLCQAHGL |
| | | TPEQVVAIASHDGGKQALETVQRLLPVLCQAHGLTPEQVVAIASNI |
| | | GGKQALETVQALLPVLCQAHGLTPQQVVAIASNNGGKQALETVQRL |
| | | EQVVAIASNIGGKQALETVQALLPVLCQAHGLTPQQVVAIASNGGG |
| | | LPVLCQAHGLTPQQVVAIASNNGGKQALETVQRLLPVLCQAHGLTP |
| | | KQALETVQRLLPVLCQAHGLTPEQVVAIASHDGGKQALETVQRLLP |
| | | VLCQAHGLTPQQVVAIASNGGGKQALETVQRLLPVLCQAHGLTPEQ |
| | | VVAIASHDGGKQALETVORLLPVLCQAHGLTPEQVVAIASNIGGKQ |
| | | ALETVQALLPVLCQAHGLTPQQVVAIASNNGGKQALETVORLLPVL |
| | | CQAHGLTPEQVVAIASHDGGKQALETVORLLPVLCQAHGLTPEQVV |
| | | AIASHDGGKQALETVQRLLPVLCQAHGLTPQQVVAIASNGGGKQAL |
| | | ETVQRLLPVLCQAHGLTPQQVVAIASNGGGRPALESIVAQLSRPDP |
| | | ALAALTNDHLVALACLGGRPALDAVKKGLGDPISRSQLVKSELEEK |
| | | KSELRHKLKYVPHEYIELIEIARNSTQDRILEMKVMEFFMKVYGYR |
| | | GKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQ |
| | | LTRLNHITNCNGAVLSVEELLIGGEMIKAGTLTLEEVRRKFNNGEI |
| | | NFAAD |
| Right LAG3 | SEQ ID NO: 34 | ATGGGCGATCCTAAAAAGAAACGTAAGGTCATCGATATCGCCGATC |
| TALEN | | TACGCACGCTCGGCTACAGCCAGCAGCAACAGGAGAAGATCAAACC |
| Nucleotide | | GAAGGTTCGTTCGACAGTGGCGCAGCACCACGAGGCACTGGTCGGC |
| sequence | | CACGGGTTTACACACGCGCACATCGTTGCGTTAAGCCAACACCCGG |
| | | CAGCGTTAGGGACCGTCGCTGTCAAGTATCAGGACATGATCGCAGC |
| | | GTTGCCAGAGGCGACACACGAAGCGATCGTTGGCGTCGGCAAACAG |
| | | TGGTCCGGCGCACGCGCTCTGGAGGCCTTGCTCACGGTGGCGGGAG |
| | | AGTTGAGAGGTCCACCGTTACAGTTGGACACAGGCCAACTTCTCAA |
| | | GATTGCAAAACGTGGCGGCGTGACCGCAGTGGAGGCAGTGCATGCA |
| | | TGGCGCAATGCACTGACGGGTGCCCCGCTCAACTTGACCCCCCAGC |
| | | AGGTGGTGGCCATCGCCAGCAATAATGGTGGCAAGCAGGCGCTGGA |
| | | GACGGTCCAGCGGCTGTTGCCGGTGCTGTGCCAGGCCCACGGCTTG |
| | | ACCCCGGAGCAGGTGGTGGCCATCGCCAGCAATATTGGTGGCAAGC |
| | | AGGCGCTGGAGACGGTGCAGGCGCTGTTGCCGGTGCTGTGCCAGGC |
| | | CCACGGCTTGACCCCCCAGCAGGTGGTGGCCATCGCCAGCAATGGC |
| | | GGTGGCAAGCAGGCGCTGGAGACGGTCCAGCGGCTGTTGCCGGTGC |
| | | TGTGCCAGGCCCACGGCTTGACCCCCCAGCAGGTGGTGGCCATCGC |
| | | CAGCAATAATGGTGGCAAGCAGGCGCTGGAGACGGTCCAGCGGCTG |
| | | TTGCCGGTGCTGTGCCAGGCCCACGGCTTGACCCCGGAGCAGGTGG |
| | | TGGCCATCGCCAGCCACGATGGCGGCAAGCAGGCGCTGGAGACGGT |
| | | CCAGCGGCTGTTGCCGGTGCTGTGCCAGGCCCACGGCTTGACCCCC |
| | | CAGCAGGTGGTGGCCATCGCCAGCAATGGCGGTGGCAAGCAGGCGC |
| | | TGGAGACGGTCCAGCGGCTGTTGCCGGTGCTGTGCCAGGCCCACGG |
| | | CTTGACCCCCCAGCAGGTGGTGGCCATCGCCAGCAATAATGGTGGC |
| | | AAGCAGGCGCTGGAGACGGTCCAGCGGCTGTTGCCGGTGCTGTGCC |
| | | AGGCCCACGGCTTGACCCCGGAGCAGGTGGTGGCCATCGCCAGCCA |
| | | CGATGGCGGCAAGCAGGCGCTGGAGACGGTCCAGCGGCTGTTGCCG |
| | | GTGCTGTGCCAGGCCCACGGCTTGACCCCGGAGCAGGTGGTGGCCA |
| | | TCGCCAGCCACGATGGCGGCAAGCAGGCGCTGGAGACGGTCCAGCG |
| | | GCTGTTGCCGGTGCTGTGCCAGGCCCACGGCTTGACCCCGGAGCAG |
| | | GTGGTGGCCATCGCCAGCAATATTGGTGGCAAGCAGGCGCTGGAGA |
| | | CGGTGCAGGCGCTGTTGCCGGTGCTGTGCCAGGCCCACGGCTTGAC |
| | | CCCGGAGCAGGTGGTGGCCATCGCCAGCAATATTGGTGGCAAGCAG |
| | | GCGCTGGAGACGGTGCAGGCGCTGTTGCCGGTGCTGTGCCAGGCCC |
| | | ACGGCTTGACCCCCCAGCAGGTGGTGGCCATCGCCAGCAATAATGG |
| | | TGGCAAGCAGGCGCTGGAGACGGTCCAGCGGCTGTTGCCGGTGCTG |
| | | TGCCAGGCCCACGGCTTGACCCCCCAGCAGGTGGTGGCCATCGCCA |
| | | GCAATGGCGGTGGCAAGCAGGCGCTGGAGACGGTCCAGCGGCTGTT |
| | | GCCGGTGCTGTGCCAGGCCCACGGCTTGACCCCCCAGCAGGTGGTG |
| | | GCCATCGCCAGCAATAATGGTGGCAAGCAGGCGCTGGAGACGGTCC |
| | | AGCGGCTGTTGCCGGTGCTGTGCCAGGCCCACGGCTTGACCCCGGA |
| | | GCAGGTGGTGGCCATCGCCAGCAATATTGGTGGCAAGCAGGCGCTG |
| | | GAGACGGTGCAGGCGCTGTTGCCGGTGCTGTGCCAGGCCCACGGCT |
| | | TGACCCCTCAGCAGGTGGTGGCCATCGCCAGCAATGGCGGCGGCAG |
| | | GCCGGCGCTGGAGAGCATTGTTGCCCAGTTATCTCGCCCTGATCCG |
| | | GCGTTGGCCGCGTTGACCAACGACCACCTCGTCGCCTTGGCCTGCC |
| | | TCGGCGGGCGTCCTGCGCTGGATGCAGTGAAAAAGGGATTGGGGGA |
| | | TCCTATCAGCCGTTCCCAGCTGGTGAAGTCCGAGCTGGAGGAGAAG |
| | | AAATCCGAGTTGAGGCACAAGCTGAAGTACGTGCCCCACGAGTACA |
| | | TCGAGCTGATCGAGATCGCCCGGAACAGCACCCAGGACCGTATCCT |
| | | GGAGATGAAGGTGATGGAGTTCTTCATGAAGGTGTACGGCTACAGG |
| | | GGCAAGCACCTGGGCGGCTCCAGGAAGCCCGACGGCGCCATCTACA |
| | | CCGTGGGCTCCCCCATCGACTACGGCGTGATCGTGGACACCAAGGC |
| | | CTACTCCGGCGGCTACAACCTGCCCATCGGCCAGGCCGACGAAATG |
| | | CAGAGGTACGTGGAGGAGAACCAGACCAGGAACAAGCACATCAACC |
| | | CCAACGAGTGGTGGAAGGTGTACCCCTCCAGCGTGACCGAGTTCAA |
| | | GTTCCTGTTCGTGTCCGGCCACTTCAAGGGCAACTACAAGGCCCAG |
| | | CTGACCAGGCTGAACCACATCACCAACTGCAACGGCGCCGTGCTGT |
| | | CCGTGGAGGAGCTCCTGATCGGCGGCGAGATGATCAAGGCCGGCAC |
| | | CCTGACCCTGGAGGAGGTGAGGAGGAAGTTCAACAACGGCGAGATC |
| | | AACTTCGCGGCCGACTGATAA |
| Right LAG3 | SEQ ID NO: 35 | MGDPKKKRKVIDIADLRTLGYSQQQQEKIKPKVRSTVAQHHEALVG |
| TALEN | | HGFTHAHIVALSQHPAALGTVAVKYQDMIAALPEATHEAIVGVGKQ |
| Amino acid | | WSGARALEALLTVAGELRGPPLQLDTGQLLKIAKRGGVTAVEAVHA |
| sequence | | WRNALTGAPLNLTPQQVVAIASNNGGKQALETVORLLPVLCQAHGL |
| | | TPEQVVAIASNIGGKQALETVQALLPVLCQAHGLTPQQVVAIASNG |
| | | GGKQALETVQRLLPVLCQAHGLTPQQVVAIASNNGGKQALETVQRL |
| | | LPVLCQAHGLTPEQVVAIASHDGGKQALETVQRLLPVLCQAHGLTP |
| | | QQVVAIASNGGGKQALETVORLLPVLCQAHGLTPQQVVAIASNNGG |
| | | KQALETVQRLLPVLCQAHGLTPEQVVAIASHDGGKQALETVORLLP |
| | | VLCQAHGLTPEQVVAIASHDGGKQALETVQRLLPVLCQAHGLTPEQ |
| | | VVAIASNIGGKQALETVQALLPVLCQAHGLTPEQVVAIASNIGGKQ |
| | | ALETVQALLPVLCQAHGLTPQQVVAIASNNGGKQALETVQRLLPVL |
| | | CQAHGLTPQQVVAIASNGGGKQALETVQRLLPVLCQAHGLTPQQVV |
| | | AIASNNGGKQALETVQRLLPVLCQAHGLTPEQVVAIASNIGGKQAL |
| | | ETVQALLPVLCQAHGLTPQQVVAIASNGGGRPALESIVAQLSRPDP |
| | | ALAALTNDHLVALACLGGRPALDAVKKGLGDPISRSQLVKSELEEK |
| | | KSELRHKLKYVPHEYIELIEIARNSTQDRILEMKVMEFFMKVYGYR |
| | | GKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQ |
| | | LTRLNHITNCNGAVLSVEELLIGGEMIKAGTLTLEEVRRKFNNGEI |
| | | NFAAD |
| LAG3 target | SEQ ID NO: 36 | TCCAGGATCTCAGCCTTctgcgaagagcagggGTCACTTGGCAGCA |
| site sequence | | TCA |

Major classes of nucleases that have been developed to enable site-specific genomic editing include transcription activator-like nucleases (TALENs), which achieve specific DNA binding via protein-DNA interactions. See, e.g., Cox et al., Nature Medicine, 2015, Vol. 21, No. 2. TALE methods, embodiments of which are described in more detail below, can be used as the gene editing method of the present invention.

As discussed above, embodiments of the present invention provide tumor infiltrating lymphocytes (TILs) that have been genetically modified via TALEN gene-editing by introducing into the TILs nucleic acids, such as mRNAs, encoding one or more TALE-nucleases to selectively inactivate by DNA cleavage a gene encoding PD-1, wherein the one or more TALE-nucleases comprise a TALE-nuclease that is directed against the nucleic acid sequence of SEQ ID NO: 18 as a PD-1 gene target sequence, and optionally by introducing into the TILs nucleic acids, such as mRNAs, encoding one or more TALE-nucleases to selectively inactivate by DNA cleavage a gene encoding TIGIT, wherein the one or more TALE-nucleases comprise a TALE-nuclease that is directed against the nucleic acid sequence of SEQ ID NO: 23 or 28 as a TIGIT gene target sequence, to enhance their therapeutic effect. Some embodiments of the present invention provide tumor infiltrating lymphocytes (TILs) that have been genetically modified via TALEN gene-editing by introducing into the TILs nucleic acids, such as mRNAs, encoding one or more TALE-nucleases to selectively inactivate by DNA cleavage a gene encoding PD-1, wherein the one or more TALE-nucleases comprise a TALE-nuclease that is directed against the nucleic acid sequence of SEQ ID NO: 18 as a PD-1 gene target sequence, and optionally by introducing into the TILs nucleic acids, such as mRNAs, encoding one or more TALE-nucleases to selectively inactivate by DNA cleavage a gene encoding LAG3, wherein the one or more TALE-nucleases comprise a TALE-nuclease that is directed against the nucleic acid sequence of SEQ ID NO: 36 as a LAG3 gene target sequence, to enhance their therapeutic effect. Embodiments of the present invention embrace methods of expansion of such genetically edited TILs into a population of TILs. Embodiments of the present invention also provide methods for expanding such genetically edited TILs into a therapeutic population.

In some embodiments, the invention provides an mRNA encoding one or more TALE-nucleases comprising the TALEN-encoding sequence, a 3′UTR sequence, and a polyA tail. In some embodiments, the invention provides an mRNA encoding one or more TALE-nucleases comprising a 3′UTR from murine HBA gene. In some embodiments, the invention provides an mRNA encoding one or more TALE-nucleases comprising a 3′ UTR having the sequence of GCTGCCTTCTGCGGGGCTTGCCTTCTGGCCATGCCCTTCTTCTCTCCCTTGCACCTGTACCTCTTGGTCTTTGAATAA AGCCTGAGTAGGAAG (SEQ ID NO: 29). In some embodiments, the invention provides an mRNA encoding one or more TALE-nucleases comprising a polyA tail, wherein the polyA tail is 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, or 100 bp long. In some embodiments, the polyA tail is 80 bp long.

a. Resting Step

In some embodiments, the two steps of sequential electroporation of TILs with nucleic acids, such as mRNAs, encoding the two TALEN systems that target PD-1 and TIGIT are separated by a resting step. According to some embodiments, the resting step comprises incubating the fourth population of TILs at about 30-40° C. with about 5% CO₂. According to some embodiments, the resting step is carried out at about 30° C., about 30.5° C., about 31° C., about 31.5° C., about 32° C., about 32.5° C., about 33° C., about 33.5° C., about 34° C., about 34.5° C., about 35° C., about 35.5° C., about 36° C., about 36.5° C., about 37° C., about 37.5° C., about 38° C., about 38.5° C., about 39° C., about 39.5° C., about 40° C. According to some embodiments, the resting step is carried out for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, or longer. According to some embodiments, the resting step comprises incubating the fourth population of TILs in a cell culture medium comprising IL-2. According to some embodiments, the resting step comprises incubating the fourth population of TILs in a cell culture medium comprising IL-2 at 300 IU/mL, 1,000 IU/mL, 2,000 IU/mL, 3,000 IU/mL, or 6,000 IU/mL. According to some embodiments, the resting step comprises incubating the fourth population of TILs in CM1 with 1,000 IU/mL IL-2. According to some embodiments, the resting step comprises incubating the third or fourth population of TILs in a cell culture medium comprising IL-2 for about 15 hours to about 23 hours at about 30° C. with about 5% CO₂. According to some embodiments, the resting step comprises incubating the fourth population of TILs in a cell culture medium comprising IL-2 for about 1 day to about 3 days at 37° C. with about 5% CO₂. According to some embodiments, the resting step comprises incubating the fourth population of TILs in a cell culture medium comprising IL-2 for about 2 days at 37° C. with about 5% CO₂.

In some embodiments, each of the two steps of sequential electroporation of TILs with nucleic acids, such as mRNAs, encoding the two TALEN systems that target PD-1 and TIGIT is followed by an overnight resting step. According to some embodiments, the overnight resting step comprises incubating the fourth or fifth population of TILs in a cell culture medium comprising IL-2 at about 25-37° C. with about 5% CO₂. According to some embodiments, the overnight resting step is carried out at about 25° C., about 25.5° C., about 26° C., about 26.5° C., about 27° C., about 27.5° C., about 28° C., about 28.5° C., about 29° C., about 29.5° C., about 30° C., about 30.5° C., about 31° C., about 31.5° C., about 32° C., about 32.5° C., about 33° C., about 33.5° C., about 34° C., about 34.5° C., about 35° C., about 35.5° C., about 36° C., about 36.5° C., and about 37° C. According to some embodiments, the overnight resting step comprises incubating the fourth or fifth population of TILs in a cell culture medium comprising IL-2 at about 30° C. with about 5% CO₂.

In some embodiments, each of the two steps of sequential electroporation of TILs with nucleic acids, such as mRNAs, encoding the two TALEN systems that target PD-1 and TIGIT is followed by an overnight resting step, separated by a resting step of about 1-3 days between the two electroporation steps. According to some embodiments, the overnight resting step comprises incubating the fourth or fifth population of TILs in a cell culture medium comprising IL-2 at about 25-37° C. with about 5% CO₂ and the resting step between the two electroporation steps comprises incubating the fourth population of TILs for about 1-3 days at about 30-40° C. with about 5% CO₂. According to some embodiments, the overnight resting step comprises incubating the fourth or fifth population of TILs in a cell culture medium comprising IL-2 at about 30° C. with about 5% CO₂ and the resting step between the two electroporation steps comprises incubating the fourth population of TILs for about 1-3 days at about 37° C. with about 5% CO₂. According to some embodiments, the overnight resting step comprises incubating the fourth or fifth population of TILs in a cell culture medium comprising IL-2 at about 30° C. with about 5% CO₂ and the resting step between the two electroporation steps comprises incubating the fourth population of TILs for about 1 day at about 37° C. with about 5% CO₂. According to some embodiments, the overnight resting step comprises incubating the fourth or fifth population of TILs in a cell culture medium comprising IL-2 at about 30° C. with about 5% CO₂ and the resting step between the two electroporation steps comprises incubating the fourth population of TILs for about 2 days at about 37° C. with about 5% CO₂. According to some embodiments, the overnight resting step comprises incubating the fourth or fifth population of TILs in a cell culture medium comprising IL-2 at about 30° C. with about 5% CO₂ and the resting step between the two electroporation steps comprises incubating the fourth population of TILs for about 3 days at about 37° C. with about 5% CO₂.

### F. Second Expansion

In some embodiments, the TIL cell population is expanded in number after initial bulk processing, pre-REP expansion, and genetic modification, wherein the expanded TILs have been genetically modified via TALEN gene editing by sequentially introducing into the TILs nucleic acids, such as mRNAs, encoding two TALEN systems that target PD-1 and TIGIT. This further expansion is referred to herein as the second expansion, which can include expansion processes generally referred to in the art as a rapid expansion process (REP). The second expansion is generally accomplished using a culture media comprising a number of components, including feeder cells, a cytokine source, and an anti-CD3 agonist antibody, in a gas-permeable container.

In some embodiments, the second expansion or second TIL expansion (which can include expansions sometimes referred to as REP) of TIL can be performed using any TIL flasks or containers known by those of skill in the art, wherein the expanded TILs have been genetically modified via TALEN gene editing by sequentially introducing into the TILs nucleic acids, such as mRNAs, encoding two TALEN systems that target PD-1 and TIGIT. In some embodiments, the second TIL expansion can proceed for 7 days, 8 days, 9 days, 10 days, 11 days, 12 days, 13 days, or 14 days. In some embodiments, the second TIL expansion can proceed for about 7 days to about 14 days. In some embodiments, the second TIL expansion can proceed for about 7 days to about 12 days. In some embodiments, the second TIL expansion can proceed for about 7 days to about 10 days. In some embodiments, the second TIL expansion can proceed for about 7 days to about 9 days. In some embodiments, the second TIL expansion can proceed for about 8 days to about 9 days. In some embodiments, the second TIL expansion can proceed for about 9 days. In some embodiments, the second TIL expansion can proceed for about 10 days. In some embodiments, the second TIL expansion can proceed for about 11 days.

In some embodiments, the second expansion can be performed in a gas permeable container using the methods of the present disclosure (including for example, expansions referred to as REP). For example, TILs can be rapidly expanded using non-specific T-cell receptor stimulation in the presence of interleukin-2 (IL-2) or interleukin-15 (IL-15). The non-specific T-cell receptor stimulus can include, for example, an anti-CD3 agonist antibody, such as about 30 ng/ml of OKT3, a mouse monoclonal anti-CD3 antibody (commercially available from Ortho-McNeil, Raritan, NJ or Miltenyi Biotech, Auburn, CA) or UHCT-1 (commercially available from BioLegend, San Diego, CA, USA). TILs can be expanded to induce further stimulation of the TILs in vitro by including one or more antigens during the second expansion, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2. In some embodiments, the re-stimulation occurs as part of the second expansion. In some embodiments, the second expansion occurs in the presence of irradiated, autologous lymphocytes or with irradiated HLA-A2+ allogeneic lymphocytes and IL-2.

In some embodiments, the cell culture medium further comprises IL-2. In some embodiments, the cell culture medium comprises about 3000 IU/mL of IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/ml of IL-2. In some embodiments, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In some embodiments, the cell culture medium comprises OKT-3 antibody. In some embodiments, the cell culture medium comprises about 30 ng/ml of OKT-3 antibody. In some embodiments, the cell culture medium comprises about 0.1 ng/ml, about 0.5 ng/ml, about 1 ng/ml, about 2.5 ng/ml, about 5 ng/ml, about 7.5 ng/ml, about 10 ng/ml, about 15 ng/ml, about 20 ng/ml, about 25 ng/ml, about 30 ng/ml, about 35 ng/ml, about 40 ng/ml, about 50 ng/ml, about 60 ng/ml, about 70 ng/ml, about 80 ng/ml, about 90 ng/ml, about 100 ng/mL, about 200 ng/ml, about 500 ng/ml, or about 1 μg/mL of OKT-3 antibody. In some embodiments, the cell culture medium comprises between 0.1 ng/ml and 1 ng/ml, between 1 ng/ml and 5 ng/ml, between 5 ng/ml and 10 ng/ml, between 10 ng/ml and 20 ng/ml, between 20 ng/ml and 30 ng/ml, between 30 ng/ml and 40 ng/mL, between 40 ng/ml and 50 ng/ml, and between 50 ng/ml and 100 ng/ml of OKT-3 antibody. In some embodiments, the cell culture medium does not comprise OKT-3 antibody. In some embodiments, the OKT-3 antibody is muromonab.

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 μg/mL and 100 μg/mL. In some embodiments, the TNFRSF agonist is added at a concentration sufficient to achieve a concentration in the cell culture medium of between 20 μg/ml and 40 μg/mL.

In some embodiments, in addition to one or more TNFRSF agonists, the cell culture medium further comprises IL-2 at an initial concentration of about 3000 IU/ml and OKT-3 antibody at an initial concentration of about 30 ng/ml, and wherein the one or more TNFRSF agonists comprises a 4-1BB agonist.

In some embodiments the antigen-presenting feeder cells (APCs) are PBMCs. In some embodiments, the ratio of TILs to PBMCs and/or antigen-presenting cells in the rapid expansion and/or the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to PBMCs in the rapid expansion and/or the second expansion is between 1 to 50 and 1 to 300. Ns % tr j % jr gtim jsyx, the ratio of TILs to PBMCs in the rapid expansion and/or the second expansion is between 1 to 100 and 1 to 200.

In some embodiments, REP and/or the second expansion is performed in flasks with the bulk TILs being mixed with a 100- or 200-fold excess of inactivated feeder cells, 30 mg/mL OKT3 anti-CD3 antibody and 3000 IU/mL IL-2 in 150 ml media. Media replacement is done (generally ⅔ media replacement via respiration with fresh media) until the cells are transferred to an alternative growth chamber. Alternative growth chambers include G-REX flasks and gas permeable containers as more fully discussed below.

In some embodiments, the second expansion (which can include processes referred to as the REP process) is shortened to 7-14 days, as discussed in the examples and figures, wherein the TILs expanded by such a second expansion have been genetically modified via TALEN gene editing by sequentially introducing into the TILs nucleic acids, such as mRNAs, encoding two TALEN systems that target PD-1 and TIGIT. In some embodiments, the second expansion is shortened to 9 days.

In some embodiments, REP and/or the second expansion may be performed using T-175 flasks and gas permeable bags as previously described (Tran, et al., J. Immunother. 2008, 31, 742-51; Dudley, et al., J. Immunother. 2003, 26, 332-42) or gas permeable cultureware (G-Rex flasks), wherein the TILs expanded by such a second expansion have been genetically modified via TALEN gene editing by sequentially introducing into the TILs nucleic acids, such as mRNAs, encoding two TALEN systems that target PD-1 and TIGIT. In some embodiments, the second expansion (including expansions referred to as rapid expansions) is performed in T-175 flasks, and about 1×10⁶ TILs suspended in 150 ml of media may be added to each T-175 flask. The TILs may be cultured in a 1 to 1 mixture of CM and AIM-V medium, supplemented with 3000 IU per mL of IL-2 and 30 ng per ml of anti-CD3. The T-175 flasks may be incubated at 37° C. in 5% CO₂, wherein the TILs expanded by such a second expansion have been genetically modified via TALEN gene editing by sequentially introducing into the TILs nucleic acids, such as mRNAs, encoding two TALEN systems that target PD-1 and TIGIT. Half the media may be exchanged on day 5 using 50/50 medium with 3000 IU per mL of IL-2. In some embodiments, on day 7 cells from two T-175 flasks may be combined in a 3 L bag and 300 ml of AIM V with 5% human AB serum and 3000 IU per mL of IL-2 was added to the 300 ml of TIL suspension. The number of cells in each bag was counted every day or two and fresh media was added to keep the cell count between 0.5 and 2.0×10⁶ cells/mL.

In some embodiments, the second expansion (which can include expansions referred to as REP) may be performed in 500 ml capacity gas permeable flasks with 100 cm gas-permeable silicon bottoms (G-Rex 100, commercially available from Wilson Wolf Manufacturing Corporation, New Brighton, MN, USA), 5×10⁶ or 10×10⁶ TIL may be cultured with PBMCs in 400 ml of 50/50 medium, supplemented with 5% human AB serum, 3000 IU per mL of IL-2 and 30 ng per ml of anti-CD3 (OKT3), wherein the TILs expanded by such a second expansion have been genetically modified via TALEN gene editing by sequentially introducing into the TILs nucleic acids, such as mRNAs, encoding two TALEN systems that target PD-1 and TIGIT. The G-Rex 100 flasks may be incubated at 37° C. in 5% CO₂. On day 5, 250 ml of supernatant may be removed and placed into centrifuge bottles and centrifuged at 1500 rpm (491×g) for 10 minutes. The TIL pellets may be re-suspended with 150 ml of fresh medium with 5% human AB serum, 3000 IU per mL of IL-2, and added back to the original G-Rex 100 flasks. When TIL are expanded serially in G-Rex 100 flasks, on day 7 the TIL in each G-Rex 100 may be suspended in the 300 ml of media present in each flask and the cell suspension may be divided into 3 100 mL aliquots that may be used to seed 3 G-Rex 100 flasks. Then 150 mL of AIM-V with 5% human AB serum and 3000 IU per mL of IL-2 may be added to each flask. The G-Rex 100 flasks may be incubated at 37° C. in 5% CO₂ and after 4 days 150 mL of AIM-V with 3000 IU per mL of IL-2 may be added to each G-REX 100 flask. The cells may be harvested on day 14 of culture.

In some embodiments, the second expansion (which can include expansions referred to as REP) may be performed in 500 ml capacity gas permeable flasks with 100 cm gas-permeable silicon bottoms (G-REX-100, commercially available from Wilson Wolf Manufacturing Corporation, New Brighton, MN, USA), 5×10⁶ or 10×10⁶ TIL may be cultured with PBMCs in 400 ml of 50/50 medium, supplemented with 5% human AB serum, 3000 IU per mL of IL-2 and 30 ng per mL of anti-CD3 (OKT3). The G-REX-100 (or G-REX100M) flasks may be incubated at 37° C. in 5% CO₂. On day 5, 250 ml of supernatant may be removed and placed into centrifuge bottles and centrifuged at 1500 rpm (491×g) for 10 minutes. The TIL pellets may be re-suspended with 150 ml of fresh medium with 5% human AB serum, 6000 IU per mL of IL-2, and added back to the original GREX-100 flasks. When TIL are expanded serially in GREX-100 flasks, on day 10 or 11 the TILs can be moved to a larger flask, such as a GREX-500 (or G-REX500M). The cells may be harvested on day 14 of culture. The cells may be harvested on day 15 of culture. The cells may be harvested on day 16 of culture. In some embodiments, media replacement is done until the cells are transferred to an alternative growth chamber. In some embodiments, ⅔ of the media is replaced by aspiration of spent media and replacement with an equal volume of fresh media. In some embodiments, alternative growth chambers include GREX flasks and gas permeable containers as more fully discussed below. In some embodiments, the process employed varying centrifugation speeds (400 g, 300 g, 200 g for 5 minutes) and varying numbers of repetitions.

In some embodiments, the second expansion (including expansions referred to as REP) is performed in flasks with the bulk TILs being mixed with a 100- or 200-fold excess of inactivated feeder cells, 30 mg/mL OKT3 anti-CD3 antibody and 3000 IU/mL IL-2 in 150 ml media. In some embodiments, media replacement is done until the cells are transferred to an alternative growth chamber, wherein the TILs expanded by such a second expansion have been genetically modified via TALEN gene editing by sequentially introducing into the TILs nucleic acids, such as mRNAs, encoding two TALEN systems that target PD-1 and TIGIT. In some embodiments, ⅔ of the media is replaced by aspiration of spent media followed by infusion with fresh media. In some embodiments, alternative growth chambers include G-REX flasks and gas permeable containers as more fully discussed below.

In some embodiments, the second expansion culture medium (e.g., sometimes referred to as CM2 or the second cell culture medium), comprises IL-2, OKT-3, as well as the antigen-presenting feeder cells (APCs), as discussed in more detail below.

In some embodiments, the second expansion is performed in a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

In some embodiments, the steps of the method are completed within a period of about 22 days. In some embodiments, the steps of the method are completed within a period of about 8 days. In some embodiments, the steps of the method are completed within a period of about 9 days. In some embodiments, the steps of the method are completed within a period of about 10 days. In some embodiments, the steps of the method are completed within a period of about 11 days. In some embodiments, the steps of the method are completed within a period of about 12 days. In some embodiments, the steps of the method are completed within a period of about 13 days. In some embodiments, the steps of the method are completed within a period of about 14 days. In some embodiments, the steps of the method are completed within a period of about 15 days. In some embodiments, the steps of the method are completed within a period of about 16 days. In some embodiments, the steps of the method are completed within a period of about 17 days. In some embodiments, the steps of the method are completed within a period of about 18 days. In some embodiments, the steps of the method are completed within a period of about 19 days. In some embodiments, the steps of the method are completed within a period of about 20 days. In some embodiments, the steps of the method are completed within a period of about 21 days. In some embodiments, the steps of the method are completed within a period of about 22 days. In some embodiments, the steps of the method are completed within a period of about 23 days. In some embodiments, the steps of the method are completed within a period of about 24 days. In some embodiments, the steps of the method are completed within a period of about 25 days. In some embodiments, the steps of the method are completed within a period of about 26 days. In some embodiments, the steps of the method are completed within a period of about 27 days. In some embodiments, the steps of the method are completed within a period of about 28 days. In some embodiments, the steps of the method are completed within a period of about 29 days. In some embodiments, the steps of the method are completed within a period of about 30 days. In some embodiments, the steps of the method are completed within a period of about 31 days.

In some embodiments, the antigen presenting cells (APCs) are PBMCs. According to some embodiments, the PBMCs are irradiated. According to some embodiments, the PBMCs are allogeneic. According to some embodiments, the PBMCs are irradiated and allogeneic. According to some embodiments, the antigen-presenting cells are artificial antigen-presenting cells.

In some embodiments, the IL-2 is present at an initial concentration of between 1000 IU/ml and 6000 IU/ml in the cell culture medium in the first expansion. In some embodiments, the IL-2 is present at an initial concentration of between 1500 IU/ml and 6000 IU/mL in the cell culture medium in the first expansion. In some embodiments, the IL-2 is present at an initial concentration of between 2000 IU/mL and 6000 IU/mL in the cell culture medium in the first expansion. In some embodiments, the IL-2 is present at an initial concentration of between 2500 IU/mL and 6000 IU/mL in the cell culture medium in the first expansion. In some embodiments, the IL-2 is present at an initial concentration of between 3000 IU/mL and 6000 IU/mL in the cell culture medium in the first expansion. In some embodiments, the IL-2 is present at an initial concentration of between 3500 IU/mL and 6000 IU/mL in the cell culture medium in the first expansion. In some embodiments, the IL-2 is present at an initial concentration of between 4000 IU/mL and 6000 IU/mL in the cell culture medium in the first expansion. In some embodiments, the IL-2 is present at an initial concentration of between 4500 IU/ml and 6000 IU/mL in the cell culture medium in the first expansion. In some embodiments, the IL-2 is present at an initial concentration of between 5000 IU/mL and 6000 IU/mL in the cell culture medium in the first expansion. In some embodiments, the IL-2 is present at an initial concentration of between 5500 IU/ml and 6000 IU/mL in the cell culture medium in the first expansion. In some embodiments, the IL-2 is present at an initial concentration of between 1000 IU/mL and 5000 IU/mL in the cell culture medium in the first expansion. In some embodiments, the IL-2 is present at an initial concentration of between 1500 IU/mL and 5000 IU/mL in the cell culture medium in the first expansion. In some embodiments, the IL-2 is present at an initial concentration of between 2000 IU/ml and 5000 IU/mL in the cell culture medium in the first expansion. In some embodiments, the IL-2 is present at an initial concentration of between 2500 IU/mL and 5000 IU/mL in the cell culture medium in the first expansion. In some embodiments, the IL-2 is present at an initial concentration of between 3000 IU/ml and 5000 IU/mL in the cell culture medium in the first expansion. In some embodiments, the IL-2 is present at an initial concentration of between 3500 IU/ml and 5000 IU/mL in the cell culture medium in the first expansion. In some embodiments, the IL-2 is present at an initial concentration of between 4000 IU/mL and 5000 IU/mL in the cell culture medium in the first expansion. In some embodiments, the IL-2 is present at an initial concentration of between 4500 IU/mL and 5000 IU/mL in the cell culture medium in the first expansion. In some embodiments, the IL-2 is present at an initial concentration of between 1000 IU/ml and 4000 IU/mL in the cell culture medium in the first expansion. In some embodiments, the IL-2 is present at an initial concentration of between 1500 IU/mL and 4000 IU/mL in the cell culture medium in the first expansion. In some embodiments, the IL-2 is present at an initial concentration of between 2000 IU/mL and 4000 IU/ml in the cell culture medium in the first expansion. In some embodiments, the IL-2 is present at an initial concentration of between 2500 IU/mL and 4000 IU/mL in the cell culture medium in the first expansion. In some embodiments, the IL-2 is present at an initial concentration of between 3000 IU/mL and 4000 IU/mL in the cell culture medium in the first expansion. In some embodiments, the IL-2 is present at an initial concentration of between 3500 IU/ml and 4000 IU/mL in the cell culture medium in the first expansion. In some embodiments, the IL-2 is present at an initial concentration of between 1000 IU/mL and 3000 IU/mL in the cell culture medium in the first expansion. In some embodiments, the IL-2 is present at an initial concentration of between 1500 IU/ml and 3000 IU/mL in the cell culture medium in the first expansion. In some embodiments, the IL-2 is present at an initial concentration of between 2000 IU/mL and 3000 IU/mL in the cell culture medium in the first expansion. In some embodiments, the IL-2 is present at an initial concentration of between 2500 IU/ml and 3000 IU/ml in the cell culture medium in the first expansion. In some embodiments, the IL-2 is present at an initial concentration of between 1000 IU/mL and 2000 IU/mL in the cell culture medium in the first expansion. In some embodiments, the IL-2 is present at an initial concentration of between 1500 IU/mL and 2000 IU/mL in the cell culture medium in the first expansion.

In some embodiments, the second expansion step, the IL-2 is present at an initial concentration of between 1000 IU/mL and 6000 IU/mL and the OKT-3 antibody is present at an initial concentration of about 30 ng/ml.

In some embodiments, the first cell culture medium and/or the second cell culture medium further comprises a 4-1BB agonist and/or an OX40 agonist.

In some embodiments, the first expansion is performed using a gas permeable container. In some embodiments, the second expansion is performed using a gas permeable container.

In some embodiments, the first cell culture medium further comprises a cytokine selected from the group consisting of IL-4, IL-7, IL-15, IL-21, and combinations thereof. In some embodiments, the second cell culture medium and/or third culture medium further comprises a cytokine selected from the group consisting of IL-4, IL-7, IL-15, IL-21, and combinations thereof.

### 1. Feeder Cells and Antigen Presenting Cells

In some embodiments, the second expansion procedures described herein require an excess of feeder cells during REP TIL expansion and/or during the second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation.

In general, the allogeneic PBMCs are inactivated, either via irradiation or heat treatment, and used in the REP procedures, as described in the examples, which provides an exemplary protocol for evaluating the replication incompetence of irradiate allogeneic PBMCs.

In some embodiments, PBMCs are considered replication incompetent and accepted for use in the TIL expansion procedures described herein if the total number of viable cells on day 14 is less than the initial viable cell number put into culture on day 0 of the REP and/or day 0 of the second expansion (i.e., the start day of the second expansion).

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30 ng/ml OKT3 antibody and 3000 IU/mL IL-2.

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 5-60 ng/mL OKT3 antibody and 1000-6000 IU/mL IL-2. In some embodiments, the PBMCs are cultured in the presence of 10-50 ng/ml OKT3 antibody and 2000-5000 IU/mL IL-2. In some embodiments, the PBMCs are cultured in the presence of 20-40 ng/ml OKT3 antibody and 2000-4000 IU/mL IL-2. In some embodiments, the PBMCs are cultured in the presence of 25-35 ng/ml OKT3 antibody and 2500-3500 IU/mL IL-2.

In some embodiments, the antigen-presenting feeder cells are PBMCs. In some embodiments, the antigen-presenting feeder cells are artificial antigen-presenting feeder cells. In some embodiments, the ratio of TILs to antigen-presenting feeder cells in the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to antigen-presenting feeder cells in the second expansion is between 1 to 50 and 1 to 300. In some embodiments, the ratio of TILs to antigen-presenting feeder cells in the second expansion is between 1 to 100 and 1 to 200.

In some embodiments, the second expansion procedures described herein require a ratio of about 2.5×10⁹ feeder cells to about 100×10⁶ TIL. In other embodiments, the second expansion procedures described herein require a ratio of about 2.5×10⁹ feeder cells to about 50×10⁶ TIL. In yet other embodiments, the second expansion procedures described herein require about 2.5×10⁹ feeder cells to about 25×10⁶ TIL.

In some embodiments, the second expansion procedures described herein require an excess of feeder cells during the second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation. In some embodiments, artificial antigen-presenting (aAPC) cells are used in place of PBMCs.

In some embodiments, artificial antigen presenting cells are used in the second expansion as a replacement for, or in combination with, PBMCs.

### 2. Cytokines and Other Additives

The expansion methods described herein generally use culture media with high doses of a cytokine, in particular IL-2, as is known in the art.

Alternatively, using combinations of cytokines for the rapid expansion and or second expansion of TILs is possible, with combinations of two or more of IL-2, IL-15 and IL-21 as is described in U.S. Patent Application Publication No. US 2017/0107490 A1, the disclosure of which is incorporated by reference herein. Thus, possible combinations include IL-2 and IL-15, IL-2 and IL-21, IL-15 and IL-21 and IL-2, IL-15 and IL-21, with the latter finding particular use in many embodiments. The use of combinations of cytokines specifically favors the generation of lymphocytes, and in particular T-cells as described therein.

### G. Harvest TILs

After the second expansion step, cells can be harvested. TILs can be harvested in any appropriate and sterile manner, including for example by centrifugation. Methods for TIL harvesting are well known in the art and any such know methods can be employed with the present process. In some embodiments, TILs are harvested using an automated system.

Cell harvesters and/or cell processing systems are commercially available from a variety of sources, including, for example, Fresenius Kabi, Tomtec Life Science, Perkin Elmer, and Inotech Biosystems International, Inc. Any cell based harvester can be employed with the present methods. In some embodiments, the cell harvester and/or cell processing systems is a membrane-based cell harvester. In some embodiments, cell harvesting is via a cell processing system, such as the LOVO system (manufactured by Fresenius Kabi). The term “LOVO cell processing system” also refers to any instrument or device manufactured by any vendor that can pump a solution comprising cells through a membrane or filter such as a spinning membrane or spinning filter in a sterile and/or closed system environment, allowing for continuous flow and cell processing to remove supernatant or cell culture media without pelletization. In some embodiments, the cell harvester and/or cell processing system can perform cell separation, washing, fluid-exchange, concentration, and/or other cell processing steps in a closed, sterile system.

In some embodiments, the harvest is performed from a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

In some embodiments, the closed system is accessed via syringes under sterile conditions in order to maintain the sterility and closed nature of the system. In some embodiments, a closed system as described in the Examples is employed.

### H. Final Formulation and Transfer to Infusion Container

After the steps as outlined in detailed above and herein are complete, TILs are transferred to a container for use in administration to a patient, such as an infusion bag or sterile vial. In some embodiments, once a therapeutically sufficient number of TILs are obtained using the expansion methods described above, they are transferred to a container, such as an infusion bag, for use in administration to a patient. In some embodiments, the TILs are cryopreserved in the infusion bag. In some embodiments, the TILs are cryopreserved prior to placement in an infusion bag. In some embodiments, the TILs are cryopreserved and not placed in an infusion bag. In some embodiments, cryopreservation is performed using a cryopreservation medium. In some embodiments, the cryopreservation media contains dimethylsulfoxide (DMSO). This is generally accomplished by putting the TIL population into a freezing solution, e.g. 85% complement inactivated AB serum and 15% dimethyl sulfoxide (DMSO). The cells in solution are placed into cryogenic vials and stored for 24 hours at −80° C., with optional transfer to gaseous nitrogen freezers for cryopreservation. See, Sadeghi, et al., Acta Oncologica 2013, 52, 978-986.

When appropriate, the cells are removed from the freezer and thawed in a 37° C. water bath until approximately ⅘ of the solution is thawed. The cells are generally resuspended in complete media and optionally washed one or more times. In some embodiments, the thawed TILs can be counted and assessed for viability as is known in the art.

In some embodiments, a population of TILs is cryopreserved using CS10 cryopreservation media (CryoStor 10, BioLife Solutions). In some embodiments, a population of TILs is cryopreserved using a cryopreservation media containing dimethylsulfoxide (DMSO). In some embodiments, a population of TILs is cryopreserved using a 1:1 (vol: vol) ratio of CS10 and cell culture media. In some embodiments, a population of TILs is cryopreserved using about a 1:1 (vol: vol) ratio of CS10 and cell culture media, further comprising additional IL-2.

In some embodiments, TILs are administered to a patient as a pharmaceutical composition. In some embodiments, the pharmaceutical composition is a suspension of TILs in a sterile buffer. TILs expanded by methods described in the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic administration.

### I. Closed Systems for TIL Manufacturing

The present invention provides for the use of closed systems during the TIL culturing process. Such closed systems allow for preventing and/or reducing microbial contamination, allow for the use of fewer flasks, and allow for cost reductions. In some embodiments, the closed system uses two containers.

Such closed systems are well-known in the art and can be found, for example, at http://www.fda.gov/cber/guidelines.htm and https://www.fda.gov/BiologicsBloodVaccines/GuidanceComplianceRegulatoryInformation/Guidances/Blood/ucm076779.htm.

Sterile connecting devices (STCDs) produce sterile welds between two pieces of compatible tubing. This procedure permits sterile connection of a variety of containers and tube diameters. In some embodiments, the closed systems include luer lock and heat-sealed systems as described in the Examples. In some embodiments, the closed system is accessed via syringes under sterile conditions in order to maintain the sterility and closed nature of the system. In some embodiments, a closed system as described in the examples is employed. In some embodiments, the TILs are formulated into a final product formulation container according to the methods described herein in the examples.

In some embodiments, the closed system uses one container from the time the tumor fragments are obtained until the TILs are ready for administration to the patient or cryopreserving. In some embodiments when two containers are used, the first container is a closed G-container (such as G-rex100M series or G-rex500M series flasks) and the population of TILs is centrifuged and transferred to an infusion bag without opening the first closed G-container. In some embodiments, when two containers are used, the infusion bag is a HypoThermosol-containing infusion bag. A closed system or closed TIL cell culture system is characterized in that once the tumor sample and/or tumor fragments have been added, the system is tightly sealed from the outside to form a closed environment free from the invasion of bacteria, fungi, and/or any other microbial contamination.

In some embodiments, the reduction in microbial contamination is between about 5% and about 100%. In some embodiments, the reduction in microbial contamination is between about 5% and about 95%. In some embodiments, the reduction in microbial contamination is between about 5% and about 90%. In some embodiments, the reduction in microbial contamination is between about 10% and about 90%. In some embodiments, the reduction in microbial contamination is between about 15% and about 85%. In some embodiments, the reduction in microbial contamination is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, about 99%, or about 100%.

The closed system allows for TIL growth in the absence and/or with a significant reduction in microbial contamination.

Moreover, pH, carbon dioxide partial pressure and oxygen partial pressure of the TIL cell culture environment each vary as the cells are cultured. Consequently, even though a medium appropriate for cell culture is circulated, the closed environment still needs to be constantly maintained as an optimal environment for TIL proliferation. To this end, it is desirable that the physical factors of pH, carbon dioxide partial pressure and oxygen partial pressure within the culture liquid of the closed environment be monitored by means of a sensor, the signal whereof is used to control a gas exchanger installed at the inlet of the culture environment, and the that gas partial pressure of the closed environment be adjusted in real time according to changes in the culture liquid so as to optimize the cell culture environment. In some embodiments, the present invention provides a closed cell culture system which incorporates at the inlet to the closed environment a gas exchanger equipped with a monitoring device which measures the pH, carbon dioxide partial pressure and oxygen partial pressure of the closed environment, and optimizes the cell culture environment by automatically adjusting gas concentrations based on signals from the monitoring device.

In some embodiments, the pressure within the closed environment is continuously or intermittently controlled. That is, the pressure in the closed environment can be varied by means of a pressure maintenance device for example, thus ensuring that the space is suitable for growth of TILs in a positive pressure state, or promoting exudation of fluid in a negative pressure state and thus promoting cell proliferation. By applying negative pressure intermittently, moreover, it is possible to uniformly and efficiently replace the circulating liquid in the closed environment by means of a temporary shrinkage in the volume of the closed environment.

In some embodiments, additional equipment, such as an electroporator (e.g., a Neon electroporator) is a component of an all-closed system. In some embodiments, optimal culture components for proliferation of the TILs can be substituted or added, and including factors such as IL-2 and/or OKT3, as well as combination, can be added.

In other embodiments, the invention provides the method described in any of the preceding paragraphs as applicable above modified such that each container recited in the method is a GREX-10. In other embodiments, the invention provides the method described in any of the preceding paragraphs as applicable above modified such that each container recited in the method is a GREX-100M. In other embodiments, the invention provides the method described in any of the preceding paragraphs as applicable above modified such that each container recited in the method is a GREX-500M.

### III. Therapeutic Population of TILs and Pharmaceutical Compositions

Embodiments of the present invention are also directed to a gene-edited population of tumor infiltrating lymphocytes (TILs) comprising an expanded population of TILs having reduced expression of PD-1 and TIGIT produced by the methods disclosed herein.

In some embodiments, the gene-edited population of TILs comprises an expanded population of TILs at least a portion of which comprises knockout of both PD-1 and TIGIT. In some embodiments, the gene-edited population of TILs comprises an expanded population of TILs about 40%, about 50%, about 60%, about 70%, about 80%, about 90%, or about 100% of which comprises knockout of both PD-1 and TIGIT. In some embodiments, the gene-edited population of TILs comprises an expanded population of TILs about 60% of which comprises knockout of both PD-1 and TIGIT. In some embodiments, the gene-edited population of TILs comprises an expanded population of TILs about 64% of which comprises knockout of both PD-1 and TIGIT.

In some embodiments, the gene-edited population of TILs comprises an expanded population of TILs having a reduction in PD-1 and/or TIGIT expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the gene-edited population of TILs comprises an expanded population of TILs having a reduction in PD-1 and/or TIGIT expression of at least about 65%, about 70%, about 75%, about 80%, about 85%, about 90%, or about 95%. In some embodiments, the gene-edited population of TILs comprises an expanded population of TILs having a reduction in PD-1 and/or TIGIT expression of at least about 75%, about 80%, about 85%, about 90%, or about 95%. In some embodiments, the gene-edited population of TILs comprises an expanded population of TILs having a reduction in PD-1 and/or TIGIT expression of at least about 80%, about 85%, about 90%, or about 95%. In some embodiments, the gene-edited population of TILs comprises an expanded population of TILs having a reduction in PD-1 and/or TIGIT expression of at least about 85%, about 90%, or about 95%. In some embodiments, the gene-edited population of TILs comprises an expanded population of TILs having a reduction in PD-1 and/or TIGIT expression of at least about 80%. In some embodiments, the gene-edited population of TILs comprises an expanded population of TILs having a reduction in PD-1 and/or TIGIT expression of at least about 85%, In some embodiments, the gene-edited population of TILs comprises an expanded population of TILs having a reduction in PD-1 and/or TIGIT expression of at least about 90%. In some embodiments, the gene-edited population of TILs comprises an expanded population of TILs having a reduction in PD-1 and/or TIGIT expression of at least about 95%. In some embodiments, the gene-edited population of TILs comprises an expanded population of TILs having a reduction in PD-1 and/or TIGIT expression of at least about 99%.

In some embodiments, the gene-edited population of TILs comprises an expanded population of TILs having a reduction in PD-1 and TIGIT with an increase in stem memory T cells (TSCMs). TSCMs are early progenitors of antigen-experienced central memory T cells. TSCMs generally display the long-term survival, self-renewal, and multipotency abilities that define stem cells, and are generally desirable for the generation of effective TIL products. TSCM have shown enhanced anti-tumor activity compared with other T cell subsets in mouse models of adoptive cell transfer. In some embodiments, the gene-edited population of TILs comprises an expanded population of TILs having a reduction in PD-1 and TIGIT results in a TIL population with a composition comprising a high proportion of TSCM. In some embodiments, the gene-edited population of TILs comprises an expanded population of TILs having a reduction in PD-1 and TIGIT with at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% increase in TSCM percentage. In some embodiments, the gene-edited population of TILs comprises an expanded population of TILs having a reduction in PD-1 and TIGIT with at least a 1-fold, 2-fold, 3-fold, 4-fold, 5-fold, or 10-fold increase in TSCMs in the TIL population. In some embodiments, the gene-edited population of TILs comprises an expanded population of TILs having a reduction in PD-1 and TIGIT with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs. In some embodiments, the gene-edited population of TILs comprises therapeutic population of TILs having a reduction in PD-1 and TIGIT with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs.

In some embodiments, the gene-edited population of TILs comprises an expanded population of TILs having a reduction in PD-1 and TIGIT with rejuvenation of antigen-experienced T-cells. In some embodiments, rejuvenation includes, for example, increased proliferation, increased T-cell activation, and/or increased antigen recognition.

In some embodiments, the gene-edited population of TILs comprises an expanded population of TILs having a reduction in PD-1 and TIGIT with enhanced anti-tumor activity. In some embodiments, the gene-edited population of TILs comprises an expanded population of TILs having a reduction in PD-1 and TIGIT with enhanced anti-tumor activity in mouse models of adoptive cell transfer. In some embodiments, the gene-edited population of TILs comprises an expanded population of TILs having a reduction in PD-1 and TIGIT with enhanced anti-tumor activity in comparison to another expanded population of TILs in mouse models of adoptive cell transfer. In some embodiments, the gene-edited population of TILs comprises an expanded population of TILs having a reduction in PD-1 and TIGIT with enhanced anti-tumor activity in comparison to another expanded population of TILs having a reduction in PD-1 only in mouse models of adoptive cell transfer. In some embodiments, the gene-edited population of TILs comprises an expanded population of TILs having a reduction in PD-1 and TIGIT with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, more anti-tumor activity in comparison to another expanded population of TILs in mouse models of adoptive cell transfer. In some embodiments, the gene-edited population of TILs comprises an expanded population of TILs having a reduction in PD-1 and TIGIT with at least 1-fold, at least 2-fold, at least 3-fold, at least 4-fold, at least 5-fold, at least 10-fold, at least 20-fold, at least 30-fold, at least 410-fold, at least 50-fold, at least 60-fold, at least 70-fold, at least 80-fold, at least 90-fold, or at least 100-fold, more anti-tumor activity in comparison to another expanded population of TILs in mouse models of adoptive cell transfer. In some embodiments, the gene-edited population of TILs comprises an expanded population of TILs having a reduction in PD-1 and TIGIT with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, more anti-tumor activity in comparison to another expanded population of TILs having a reduction in TIGIT only in mouse models of adoptive cell transfer. In some embodiments, the gene-edited population of TILs comprises an expanded population of TILs having a reduction in PD-1 and TIGIT with at least 1-fold, at least 2-fold, at least 3-fold, at least 4-fold, at least 5-fold, at least 10-fold, at least 20-fold, at least 30-fold, at least 410-fold, at least 50-fold, at least 60-fold, at least 70-fold, at least 80-fold, at least 90-fold, or at least 100-fold, more anti-tumor activity in comparison to another expanded population of TILs having a reduction in TIGIT only in mouse models of adoptive cell transfer. In some embodiments, the gene-edited population of TILs comprises an expanded population of TILs having a reduction in PD-1 and TIGIT with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, more anti-tumor activity in comparison to another expanded population of TILs having a reduction in PD-1 only in mouse models of adoptive cell transfer. In some embodiments, the gene-edited population of TILs comprises an expanded population of TILs having a reduction in PD-1 and TIGIT with at least 1-fold, at least 2-fold, at least 3-fold, at least 4-fold, at least 5-fold, at least 10-fold, at least 20-fold, at least 30-fold, at least 410-fold, at least 50-fold, at least 60-fold, at least 70-fold, at least 80-fold, at least 90-fold, or at least 100-fold, more anti-tumor activity in comparison to another expanded population of TILs having a reduction in PD-1 only in mouse models of adoptive cell transfer.

In some embodiments, the gene-edited population of TILs comprises a therapeutic effective dosage of TILs having reduced expression of PD-1 and TIGIT. In some embodiments, the number of the TILs for a therapeutic effective dosage of TILs is, is about, is less than, is more than, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, or a range between any two of the above values. In some embodiments, the number of the TILs for a therapeutic effective dosage of TILs is in the range of about 1×10⁶ to about 5×10⁶, about 5×10⁶ to about 1×10⁷, about 1×10⁷ to about 5×10⁷, about 5×10⁷ to about 1×10⁸, about 1×10⁸ to about 5×10⁸, about 5×10⁸ to about 1×10⁹, about 1×10⁹ to about 5×10⁹, about 5×10⁹ to about 1×10¹⁰, about 1×10¹⁰ to about 5×10¹⁰, about 5×10¹⁰ to about 1×10¹¹, about 5×10¹¹ to about 1×10¹², about 1×10¹² to about 5×10¹², and about 5×10¹² to about 1×10¹³. In some embodiments, the number of the TILs for a therapeutic effective dosage of TILs is in the range of about 1×10⁹ to about 1×10¹³. In some embodiments, the number of the TILs for a therapeutic effective dosage of TILs is in the range of about 1×10⁹ to about 1×10¹¹.

In some embodiments, the gene-edited population of TILs comprises an expanded population of TILs having reduced expression of PD-1 and TIGIT produced by the methods disclosed herein are administered to a patient as a pharmaceutical composition. In some embodiments, the pharmaceutical composition is a suspension of TILs in a sterile buffer. TILs having reduced expression of PD-1 and TIGIT produced by the methods disclosed herein may be administered by any suitable route as known in the art. In some embodiments, the TILs having reduced expression of PD-1 and TIGIT produced by the methods disclosed herein are administered as a single intra-arterial or intravenous infusion, which preferably lasts approximately 30 to 60 minutes. Other suitable routes of administration include intraperitoneal, intrathecal, and intralymphatic administration.

Any suitable dose of TILs having reduced expression of PD-1 and TIGIT produced by the methods disclosed herein can be administered. In some embodiments, from about 2.3×10¹⁰ to about 13.7×10¹⁰ TILs are administered, with an average of around 7.8×10¹⁰ TILs. In some embodiments, from about 2.3×10¹⁰ to about 13.7×10¹⁰ TILs are administered, with an average of around 7.8×10¹⁰ TILs. In some embodiments, about 1.2×10¹⁰ to about 4.3×10¹⁰ of TILs are administered. In some embodiments, about 3×10¹⁰ to about 12×10¹⁰ TILs are administered. In some embodiments, about 4×10¹⁰ to about 10×10¹⁰ TILs are administered. In some embodiments, about 5×10¹⁰ to about 8×10¹⁰ TILs are administered. In some embodiments, about 6×10¹⁰ to about 8×10¹⁰ TILs are administered. In some embodiments, about 7×10¹⁰ to about 8×10¹⁰ TILs are administered.

In some embodiments, the concentration of the TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TILs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

In some embodiments, TILs may be administered in a single dose. Such administration may be by injection, e.g., intravenous injection. In some embodiments, TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation, or by inhalation.

In other embodiments, the invention provides an infusion bag comprising the therapeutic population of TILs described in any of the preceding paragraphs above.

In other embodiments, the invention provides a tumor infiltrating lymphocyte (TIL) composition comprising the therapeutic population of TILs described in any of the preceding paragraphs above and a pharmaceutically acceptable carrier.

In other embodiments, the invention provides an infusion bag comprising the TIL composition described in any of the preceding paragraphs above.

In other embodiments, the invention provides a cryopreserved preparation of the therapeutic population of TILs described in any of the preceding paragraphs above.

In other embodiments, the invention provides a tumor infiltrating lymphocyte (TIL) composition comprising the therapeutic population of TILs described in any of the preceding paragraphs above and a cryopreservation media.

In other embodiments, the invention provides the TIL composition described in any of the preceding paragraphs above modified such that the cryopreservation media contains DMSO.

In other embodiments, the invention provides the TIL composition described in any of the preceding paragraphs above modified such that the cryopreservation media contains 7-10% DMSO.

In other embodiments, the invention provides a cryopreserved preparation of the TIL composition described in any of the preceding paragraphs above.

In some embodiments, TILs expanded using the methods of the present disclosure are administered to a patient as a pharmaceutical composition. In some embodiments, the pharmaceutical composition is a suspension of TILs in a sterile buffer. TILs expanded using methods of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic administration.

The TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

### IV. Methods of Treating Cancer Patients

Embodiments of the present invention are further directed to a method for treating a cancer patient, the method comprising administering a therapeutically effective dose of the gene-edited population of tumor infiltrating lymphocytes (TILs) comprising an expanded population of TILs having reduced expression of PD-1 and TIGIT produced by the methods disclosed herein, or the pharmaceutical composition disclosed herein, to the cancer patient.

In some embodiments, the cancer is a solid tumor cancer. In some embodiments, the solid tumor cancer is selected from the group consisting of anal cancer, bladder cancer, breast cancer (including triple-negative breast cancer), bone cancer, cancer caused by human papilloma virus (HPV), central nervous system associated cancer (including ependymoma, medulloblastoma, neuroblastoma, pineoblastoma, and primitive neuroectodermal tumor), cervical cancer (including squamous cell cervical cancer, adenosquamous cervical cancer, and cervical adenocarcinoma), colon cancer, colorectal cancer, endometrial cancer, esophageal cancer, esophagogastric junction cancer, gastric cancer, gastrointestinal cancer, gastrointestinal stromal tumor, glioblastoma, glioma, head and neck cancer (including head and neck squamous cell carcinoma (HNSCC), hypopharynx cancer, larynx cancer, nasopharynx cancer, oropharynx cancer, and pharynx cancer), kidney cancer, liver cancer, lung cancer (including non-small-cell lung cancer (NSCLC), metastatic NSCLC, and small-cell lung cancer), melanoma (including uveal melanoma, choroidal melanoma, ciliary body melanoma, iris melanoma, or metastatic melanoma), mesothelioma (including malignant pleural mesothelioma), ovarian cancer, pancreatic cancer (including pancreatic ductal adenocarcinoma), penile cancer, rectal cancer, renal cancer, renal cell carcinoma, sarcoma (including Ewing sarcoma, osteosarcoma, rhabdomyosarcoma, and other bone and soft tissue sarcomas), thyroid cancer (including anaplastic thyroid cancer), uterine cancer, and vaginal cancer.

In some embodiments, the cancer is a hematological malignancy. In some embodiments, the hematological malignancy is selected from the group consisting of chronic lymphocytic leukemia, acute lymphoblastic leukemia, diffuse large B cell lymphoma, non-Hodgkin's lymphoma, Hodgkin's lymphoma, follicular lymphoma, mantle cell lymphoma, and multiple myeloma.

In some embodiments, the cancer is one of the foregoing cancers, including solid tumor cancers and hematological malignancies, that is relapsed or refractory to treatment with at least one prior therapy, including chemotherapy, radiation therapy, or immunotherapy. In some embodiments, the cancer is one of the foregoing cancers that is relapsed or refractory to treatment with at least two prior therapies, including chemotherapy, radiation therapy, and/or immunotherapy. In some embodiments, the cancer is one of the foregoing cancers that is relapsed or refractory to treatment with at least three prior therapies, including chemotherapy, radiation therapy, and/or immunotherapy.

In other embodiments, the invention provides the method for treating a subject with cancer described herein modified such that prior to administering the therapeutically effective dosage of the therapeutic TIL population and the TIL composition described herein, respectively, a non-myeloablative lymphodepletion regimen has been administered to the subject.

In other embodiments, the invention provides the method for treating a subject with cancer described herein modified such that the non-myeloablative lymphodepletion regimen comprises the steps of administration of cyclophosphamide at a dose of 60 mg/m2/day for two days followed by administration of fludarabine at a dose of 25 mg/m2/day for five days.

In other embodiments, the invention provides the method for treating a subject with cancer described herein modified to further comprise the step of treating the subject with a high-dose IL-2 regimen starting on the day after administration of the TIL cells to the subject.

In other embodiments, the invention provides the method for treating a subject with cancer described herein modified such that the high-dose IL-2 regimen comprises 600,000 or 720,000 IU/kg administered as a 15-minute bolus intravenous infusion every eight hours until tolerance.

### 1. Lymphodepletion Preconditioning of Patients

In some embodiments, the invention includes a method of treating a cancer with a population of TILs, wherein a patient is pre-treated with non-myeloablative chemotherapy prior to an infusion of TILs according to the present disclosure. In some embodiments, the invention includes a population of TILs for use in the treatment of cancer in a patient which has been pre-treated with non-myeloablative chemotherapy. In some embodiments, the population of TILs is for administration by infusion. In some embodiments, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In some embodiments, after non-myeloablative chemotherapy and TIL infusion (at day 0) according to the present disclosure, the patient receives an intravenous infusion of IL-2 (aldesleukin, commercially available as PROLEUKIN) intravenously at 720,000 IU/kg every 8 hours to physiologic tolerance. In certain embodiments, the population of TILs is for use in treating cancer in combination with IL-2, wherein the IL-2 is administered after the population of TILs.

Experimental findings indicate that lymphodepletion prior to adoptive transfer of tumor-specific T lymphocytes plays a key role in enhancing treatment efficacy by eliminating regulatory T cells and competing elements of the immune system (‘cytokine sinks’). Accordingly, some embodiments of the invention utilize a lymphodepletion step (sometimes also referred to as “immunosuppressive conditioning”) on the patient prior to the introduction of the TILs of the invention.

In general, lymphodepletion is achieved using administration of fludarabine or cyclophosphamide (the active form being referred to as mafosfamide) and combinations thereof. Such methods are described in Gassner, et al., Cancer Immunol. Immunother. 2011, 60, 75-85, Muranski, et al., Nat. Clin. Pract. Oncol., 2006, 3, 668-681, Dudley, et al., J. Clin. Oncol. 2008, 26, 5233-5239, and Dudley, et al., J. Clin. Oncol. 2005, 23, 2346-2357, all of which are incorporated by reference herein in their entireties.

In some embodiments, the fludarabine is administered at a concentration of 0.5 μg/ml to 10 μg/mL fludarabine. In some embodiments, the fludarabine is administered at a concentration of 1 μg/mL fludarabine. In some embodiments, the fludarabine treatment is administered for 1 day, 2 days, 3 days, 4 days, 5 days, 6 days, or 7 days or more. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, the mafosfamide, the active form of cyclophosphamide, is obtained at a concentration of 0.5 μg/mL to 10 μg/mL by administration of cyclophosphamide. In some embodiments, mafosfamide, the active form of cyclophosphamide, is obtained at a concentration of 1 μg/mL by administration of cyclophosphamide. In some embodiments, the cyclophosphamide treatment is administered for 1 day, 2 days, 3 days, 4 days, 5 days, 6 days, or 7 days or more. In some embodiments, the cyclophosphamide is administered at a dosage of 100 mg/m²/day, 150 mg/m²/day, 175 mg/m²/day, 200 mg/m²/day, 225 mg/m²/day, 250 mg/m²/day, 275 mg/m²/day, or 300 mg/m²/day. In some embodiments, the cyclophosphamide is administered intravenously (i.e., i.v.) In some embodiments, the cyclophosphamide treatment is administered for 2-7 days at 35 mg/kg/day. In some embodiments, the cyclophosphamide treatment is administered for 4-5 days at 250 mg/m²/day i.v. In some embodiments, the cyclophosphamide treatment is administered for 4 days at 250 mg/m²/day i.v.

In some embodiments, lymphodepletion is performed by administering the fludarabine and the cyclophosphamide together to a patient. In some embodiments, fludarabine is administered at 25 mg/m²/day i.v. and cyclophosphamide is administered at 250 mg/m²/day i.v. over 4 days.

In some embodiments, the lymphodepletion is performed by administration of cyclophosphamide at a dose of 60 mg/m²/day for two days followed by administration of fludarabine at a dose of 25 mg/m²/day for five days.

In some embodiments, the lymphodepletion is performed by administration of cyclophosphamide at a dose of 60 mg/m²/day for two days and administration of fludarabine at a dose of 25 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 50 mg/m²/day for two days and administration of fludarabine at a dose of about 25 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 50 mg/m²/day for two days and administration of fludarabine at a dose of about 20 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 40 mg/m²/day for two days and administration of fludarabine at a dose of about 20 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 40 mg/m²/day for two days and administration of fludarabine at a dose of about 15 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of 60 mg/m²/day and fludarabine at a dose of 25 mg/m²/day for two days followed by administration of fludarabine at a dose of 25 mg/m²/day for three days.

In some embodiments, the cyclophosphamide is administered with mesna. In some embodiments, mesna is administered at 15 mg/kg. In some embodiments where mesna is infused, and if infused continuously, mesna can be infused over approximately 2 hours with cyclophosphamide (on Days-5 and/or -4), then at a rate of 3 mg/kg/hour for the remaining 22 hours over the 24 hours starting concomitantly with each cyclophosphamide dose.

In some embodiments, the lymphodepletion comprises the step of treating the patient with an IL-2 regimen starting on the day after administration of the third population of TILs to the patient.

In some embodiments, the lymphodepletion comprises the step of treating the patient with an IL-2 regimen starting on the same day as administration of the third population of TILs to the patient.

In some embodiments, the lymphodeplete comprises 5 days of preconditioning treatment. In some embodiments, the days are indicated as days −5 through −1, or Day 0 through Day 4. In some embodiments, the regimen comprises cyclophosphamide on days −5 and −4 (i.e., days 0 and 1). In some embodiments, the regimen comprises intravenous cyclophosphamide on days −5 and −4 (i.e., days 0 and 1). In some embodiments, the regimen comprises 60 mg/kg intravenous cyclophosphamide on days −5 and −4 (i.e., days 0 and 1). In some embodiments, the cyclophosphamide is administered with mesna. In some embodiments, the regimen further comprises fludarabine. In some embodiments, the regimen further comprises intravenous fludarabine. In some embodiments, the regimen further comprises 25 mg/m² intravenous fludarabine. In some embodiments, the regimen further comprises 25 mg/m² intravenous fludarabine on days −5 and −1 (i.e., days 0 through 4). In some embodiments, the regimen further comprises 25 mg/m² intravenous fludarabine on days −5 and -1 (i.e., days 0 through 4).

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for five days.

In some embodiments, the non-myeloablative lymphodepletion regimen comprises the steps of administration of cyclophosphamide at a dose of 60 mg/m²/day for two days followed by administration of fludarabine at a dose of 25 mg/m²/day for five days.

In some embodiments, the non-myeloablative lymphodepletion regimen comprises the steps of administration of cyclophosphamide at a dose of 60 mg/m²/day for two days followed by administration of fludarabine at a dose of 25 mg/m²/day for three days.

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for one day.

In some embodiments, the non-myeloablative lymphodepletion regimen comprises the steps of administration of cyclophosphamide at a dose of 60 mg/m²/day for two days followed by administration of fludarabine at a dose of 25 mg/m²/day for three days.

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

In some embodiments, the non-myeloablative lymphodepletion regimen is administered according to Table 26.

TABLE 5
Exemplary lymphodepletion and treatment regimen.

| | | | | | | | | | | |
|---|---|---|---|---|---|---|---|---|---|---|
| Day | −5 | −4 | −3 | −2 | −1 | 0 | 1 | 2 | 3 | 4 |

| | | | | | | |
|---|---|---|---|---|---|---|
| Cyclophosphamide | X | X | | | | |
| 60 mg/kg | | | | | | |
| Mesna (as needed) | X | X | | | | |
| Fludarabine | X | X | X | X | X | |
| 25 mg/m²/day | | | | | | |
| TIL infusion | | | | | | X |

In some embodiments, the non-myeloablative lymphodepletion regimen is administered according to Table 27.

TABLE 6
Exemplary lymphodepletion and treatment regimen.

| | | | | | | | | | |
|---|---|---|---|---|---|---|---|---|---|
| Day | −4 | −3 | −2 | −1 | 0 | 1 | 2 | 3 | 4 |

| | | | | | |
|---|---|---|---|---|---|
| Cyclophosphamide 60 mg/kg | X | X | | | |
| Mesna (as needed) | X | X | | | |
| Fludarabine 25 mg/m²/day | X | X | X | X | |
| TIL infusion | | | | | X |

In some embodiments, the non-myeloablative lymphodepletion regimen is administered according to Table 28.

TABLE 7
Exemplary lymphodepletion and treatment regimen.

| | | | | | | | | |
|---|---|---|---|---|---|---|---|---|
| Day | −3 | −2 | −1 | 0 | 1 | 2 | 3 | 4 |

| | | | | |
|---|---|---|---|---|
| Cyclophosphamide 60 mg/kg | X | X | | |
| Mesna (as needed) | X | X | | |
| Fludarabine 25 mg/m²/day | X | X | X | |
| TIL infusion | | | | X |

In some embodiments, the non-myeloablative lymphodepletion regimen is administered according to Table 29.

TABLE 8
Exemplary lymphodepletion and treatment regimen.

| | | | | | | | | | | |
|---|---|---|---|---|---|---|---|---|---|---|
| Day | −5 | −4 | −3 | −2 | −1 | 0 | 1 | 2 | 3 | 4 |

| | | | | | | |
|---|---|---|---|---|---|---|
| Cyclophosphamide | X | X | | | | |
| 60 mg/kg | | | | | | |
| Mesna (as needed) | X | X | | | | |
| Fludarabine | | | X | X | X | |
| 25 mg/m²/day | | | | | | |
| TIL infusion | | | | | | X |

In some embodiments, the non-myeloablative lymphodepletion regimen is administered according to Table 30.

TABLE 9
Exemplary lymphodepletion and treatment regimen.

| | | | | | | | | | | |
|---|---|---|---|---|---|---|---|---|---|---|
| Day | −5 | −4 | −3 | −2 | −1 | 0 | 1 | 2 | 3 | 4 |

| | | | | | | |
|---|---|---|---|---|---|---|
| Cyclophosphamide | X | X | | | | |
| 300 mg/kg | | | | | | |
| Mesna (as needed) | X | X | | | | |
| Fludarabine | X | X | X | X | X | |
| 30 mg/m²/day | | | | | | |
| TIL infusion | | | | | | X |

In some embodiments, the non-myeloablative lymphodepletion regimen is administered according to Table 31.

TABLE 10
Exemplary lymphodepletion and treatment regimen.

| | | | | | | | | | |
|---|---|---|---|---|---|---|---|---|---|
| Day | −4 | −3 | −2 | −1 | 0 | 1 | 2 | 3 | 4 |

| | | | | | |
|---|---|---|---|---|---|
| Cyclophosphamide 300 mg/kg | X | X | | | |
| Mesna (as needed) | X | X | | | |
| Fludarabine 30 mg/m²/day | X | X | X | X | |
| TIL infusion | | | | | X |

In some embodiments, the non-myeloablative lymphodepletion regimen is administered according to Table 32.

TABLE 11
Exemplary lymphodepletion and treatment regimen.

| | | | | | | | | |
|---|---|---|---|---|---|---|---|---|
| Day | −3 | −2 | −1 | 0 | 1 | 2 | 3 | 4 |

| | | | | |
|---|---|---|---|---|
| Cyclophosphamide 300 mg/kg | X | X | | |
| Mesna (as needed) | X | X | | |
| Fludarabine 30 mg/m²/day | X | X | X | |
| TIL infusion | | | | X |

In some embodiments, the non-myeloablative lymphodepletion regimen is administered according to Table 33.

TABLE 12
Exemplary lymphodepletion and treatment regimen.

| | | | | | | | | | | |
|---|---|---|---|---|---|---|---|---|---|---|
| Day | −5 | −4 | −3 | −2 | −1 | 0 | 1 | 2 | 3 | 4 |

| | | | | | | |
|---|---|---|---|---|---|---|
| Cyclophosphamide | X | X | | | | |
| 300 mg/kg | | | | | | |
| Mesna (as needed) | X | X | | | | |
| Fludarabine | | | X | X | X | |
| 30 mg/m²/day | | | | | | |
| TIL infusion | | | | | | X |

In some embodiments, the TIL infusion used with the foregoing embodiments of myeloablative lymphodepletion regimens may be any TIL composition described herein, as well as the addition of IL-2 regimens as described herein.

### 2. IL-2 Regimens

In some embodiments, the IL-2 regimen comprises a high-dose IL-2 regimen, wherein the high-dose IL-2 regimen comprises aldesleukin, or a biosimilar or variant thereof, administered intravenously starting on the day after administering a therapeutically effective portion of the therapeutic population of TILs, wherein the aldesleukin or a biosimilar or variant thereof is administered at a dose of 0.037 mg/kg or 0.044 mg/kg IU/kg (patient body mass) using 15-minute bolus intravenous infusions every eight hours until tolerance, for a maximum of 14 doses. Following 9 days of rest, this schedule may be repeated for another 14 doses, for a maximum of 28 doses in total. In some embodiments, IL-2 is administered in 1, 2, 3, 4, 5, or 6 doses. In some embodiments, IL-2 is administered at a maximum dosage of up to 6 doses.

In some embodiments, the IL-2 regimen comprises a decrescendo IL-2 regimen. Decrescendo IL-2 regimens have been described in O'Day, et al., J. Clin. Oncol. 1999, 17, 2752-61 and Eton, et al., Cancer 2000, 88, 1703-9, the disclosures of which are incorporated herein by reference. In some embodiments, a decrescendo IL-2 regimen comprises 18×10⁶ IU/m² aldesleukin, or a biosimilar or variant thereof, administered intravenously over 6 hours, followed by 18×10⁶ IU/m² administered intravenously over 12 hours, followed by 18×10⁶ IU/m² administered intravenously over 24 hours, followed by 4.5×10⁶ IU/m² administered intravenously over 72 hours. This treatment cycle may be repeated every 28 days for a maximum of four cycles. In some embodiments, a decrescendo IL-2 regimen comprises 18,000,000 IU/m² on day 1, 9,000,000 IU/m² on day 2, and 4,500,000 IU/m² on days 3 and 4.

In some embodiments, the IL-2 regimen comprises a low-dose IL-2 regimen. Any low-dose IL-2 regimen known in the art may be used, including the low-dose IL-2 regimens described in Dominguez-Villar and Hafler, Nat. Immunology 2000, 19, 665-673; Hartemann, et al., Lancet Diabetes Endocrinol. 2013, 1, 295-305; and Rosenzwaig, et al., Ann. Rheum. Dis. 2019, 78, 209-217, the disclosures of which are incorporated herein by reference. In some embodiments, a low-dose IL-2 regimen comprises 18×10⁶ IU per m² of aldesleukin, or a biosimilar or variant thereof, per 24 hours, administered as a continuous infusion for 5 days, followed by 2-6 days without IL-2 therapy, optionally followed by an additional 5 days of intravenous aldesleukin or a biosimilar or variant thereof, as a continuous infusion of 18×10⁶ IU per m² per 24 hours, optionally followed by 3 weeks without IL-2 therapy, after which additional cycles may be administered.

In some embodiments, IL-2 is administered at a maximum dosage of up to 6 doses. In some embodiments, the high-dose IL-2 regimen is adapted for pediatric use. In some embodiments, a dose of 600,000 international units (IU)/kg of aldesleukin every 8-12 hours for up to a maximum of 6 doses is used. In some embodiments, a dose of 500,000 international units (IU)/kg of aldesleukin every 8-12 hours for up to a maximum of 6 doses is used. In some embodiments, a dose of 400,000 international units (IU)/kg of aldesleukin every 8-12 hours for up to a maximum of 6 doses is used. In some embodiments, a dose of 500,000 international units (IU)/kg of aldesleukin every 8-12 hours for up to a maximum of 6 doses is used. In some embodiments, a dose of 300,000 international units (IU)/kg of aldesleukin every 8-12 hours for up to a maximum of 6 doses is used. In some embodiments, a dose of 200,000 international units (IU)/kg of aldesleukin every 8-12 hours for up to a maximum of 6 doses is used. In some embodiments, a dose of 100,000 international units (IU)/kg of aldesleukin every 8-12 hours for up to a maximum of 6 doses is used.

In some embodiments, the IL-2 regimen comprises administration of pegylated IL-2 every 1, 2, 4, 6, 7, 14 or 21 days at a dose of 0.10 mg/day to 50 mg/day. In some embodiments, the IL-2 regimen comprises administration of bempegaldesleukin, or a fragment, variant, or biosimilar thereof, every 1, 2, 4, 6, 7, 14 or 21 days at a dose of 0.10 mg/day to 50 mg/day.

In some embodiments, the IL-2 regimen comprises administration of THOR-707, or a fragment, variant, or biosimilar thereof, every 1, 2, 4, 6, 7, 14 or 21 days at a dose of 0.10 mg/day to 50 mg/day.

In some embodiments, the IL-2 regimen comprises administration of nemvaleukin alfa, or a fragment, variant, or biosimilar thereof, following administration of TIL. In certain embodiments, the patient the nemvaleukin is administered every 1, 2, 4, 6, 7, 14 or 21 days at a dose of 0.10 mg/day to 50 mg/day.

In some embodiments, the antibody cytokine engrafted protein described herein has a longer serum half-life that a wild-type IL-2 molecule such as, but not limited to, aldesleukin (Proleukin®) or a comparable molecule.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Preparation of Media for Pre-Rep and Rep Processes

This example describes the procedure for the preparation of tissue culture media for use in protocols involving the culture of tumor infiltrating lymphocytes (TIL) derived from various solid tumors. This media can be used for preparation of any of the TILs described in the present application and other examples.

Preparation of CM1. Removed the following reagents from cold storage and warm them in a 37° C. water bath: (RPMI1640, Human AB serum, 200 mM L-glutamine). Prepared CM1 medium according to Table 42 below by adding each of the ingredients into the top section of a 0.2 μm filter unit appropriate to the volume to be filtered. Store at 4° C.

TABLE 13
Preparation of CM1

| | Final | | Final Volume | Final |
|---|---|---|---|---|
| Ingredient | concentration | | 500 mL | Volume IL |
| RPMI1640 | NA | | 450 mL | 900 mL |
| Human AB serum, | 50 | mL | 100 mL | |
| heat-inactivated 10% | | | | |
| 200 mM L-glutamine | 2 | mM | 5 mL | 10 mL |
| 55 mM BME | 55 | μM | 0.5 mL | 1 mL |
| 50 mg/ml gentamicin | 50 | μg/mL | 0.5 mL | 1 mL |

sulfate

On the day of use, prewarmed required amount of CM1 in 37° C. water bath and add 6000 IU/mL IL-2.

Additional supplementation may be performed as needed according to Table 43.

TABLE 14
Additional supplementation of CM1, as needed.

| Supplement | Stock concentration | | Dilution | Final concentration | |
|---|---|---|---|---|---|
| GlutaMAX ™ | 200 | mM | 1:100 | 2 | mM |
| Penicillin/ | 10,000 U/mL penicillin | | 1:100 | 100 U/mL penicillin | |
| streptomycin | 10,000 μg/mL | | | 100 μg/mL | |
| | streptomycin | | | streptomycin | |
| Amphotericin | 250 | μg/mL | 1:100 | 2.5 | μg/mL |
| B | | | | | |

### Preparation of CM2

Removed prepared CM1 from refrigerator or prepare fresh CM1. Removed AIM-V® from refrigerator and prepared the amount of CM2 needed by mixing prepared CM1 with an equal volume of AIM-V® in a sterile media bottle. Added 3000 IU/mL IL-2 to CM2 medium on the day of usage. Made sufficient amount of CM2 with 3000 IU/mL IL-2 on the day of usage. Labeled the CM2 media bottle with its name, the initials of the preparer, the date it was filtered/prepared, the two-week expiration date and store at 4° C. until needed for tissue culture.

### Preparation of CM3

Prepared CM3 on the day it was required for use. CM3 was the same as AIM-V® medium, supplemented with 3000 IU/mL IL-2 on the day of use. Prepared an amount of CM3 sufficient to experimental needs by adding IL-2 stock solution directly to the bottle or bag of AIM-V. Mixed well by gentle shaking. Label bottle with “3000 IU/mL IL-2” immediately after adding to the AIM-V. If there was excess CM3, stored it in bottles at 4° C. labeled with the media name, the initials of the preparer, the date the media was prepared, and its expiration date (7 days after preparation). Discarded media supplemented with IL-2 after 7 days storage at 4° C.

### Preparation of CM4

CM4 was the same as CM3, with the additional supplement of 2 mM GlutaMAX™ (final concentration). For every 1 L of CM3, add 10 ml of 200 mM GlutaMAX™. Prepare an amount of CM4 sufficient to experimental needs by adding IL-2 stock solution and GlutaMAX™ stock solution directly to the bottle or bag of AIM-V. Mixed well by gentle shaking. Labeled bottle with “3000 IL/ml IL-2 and GlutaMAX” immediately after adding to the AIM-V. If there was excess CM4, stored it in bottles at 4° C. labeled with the media name, “GlutaMAX”, and its expiration date (7 days after preparation). Discarded media supplemented with IL-2 after more than 7-days storage at 4° C.

### Example 2: Comparison of Concomitant Vs. Sequential Electroporation

### Methods

8 mL of a 300 ng/mL OKT3 solution was prepared in Carbonate/Bicarbonate Buffer. 300 ul of the OKT3 solution was added to each well of a Nunclon 24-well TC plate and incubated overnight at 4° C.

Pre-REP TIL lines (N=2) from different indications (head & neck and breast) were thawed in CM1 with 300 IU/mL IL-2, activated in the OKT3 coated 24-well plate at 2e6 cells per well, and incubated at 37° C. for 2 days. The activated TILs were combined to more than 14e6 live cells, span down, and resuspended at 50e6/mL in Electroporation Medium T.

PD-1 and LAG3 TALEN mRNA was prepared according to the following conditions:

| | | LAG3 | PD-1 | LAG3 + PD-1 |
|---|---|---|---|---|
| | | (4 ug/1e6 cells) | (4 ug/1e6 cells) | (4 ug/1e6 cells) |
| LAG3 L | 0.9 ug/uL | 8.9 uL | | 8.9 uL |
| LAG3 R | 0.5 ug/uL | 16 uL | | 16 uL |
| PD-1 L | 2 ug/uL | | 4 uL | 4 uL |
| PD-1 R | 2 ug/uL | | 4 uL | 4 uL |

A 24 well plate was prepared with 2 mL of CM1+1000 IU/mL IL-2 in each well and kept at 30° C. at least 30 minutes before starting of electroporation. BTX Electroporator was set up to run the following protocol:

| | Group 1 | Group 2 | Group 3 |
|---|---|---|---|
| Amplitude (in V) | 200 | 200 | 130 |
| Duration (in ms) | 0.1 | 0.1 | 0.2 |
| Internal (in ms) | 0.2 | 100 | 2 |
| Number | 1 | 1 | 4 |

TILs were electroporated with either LAG3 TAL, PD-1 TAL, or LAG3+PD-1 TAL. For sequential electroporation, the TILs were electroporated with LAG3 TAL, rested overnight at 30° C. and then at 37° C. for 2 days (total 3 day rest time) in CM1 with 1,000 IU/mL IL-2, then electroporated again with PD-1 TAL.

After electroporation, the cells were incubated overnight at 30° C. in CM1 with 1,000 IU/mL IL-2. The concomitantly or sequentially electroporated TILs were incubated overnight at 30° C. in CM1 with 1,000 IU/mL IL-2, then REP′d using 3 mL of CM2, 3000 IU/mL IL-2, 30 ng/ml OKT3, and 30e6 feeders per well of a GREX 24-well plate. On Day 5 of the REP, 5 mL of CM4+3000 IU/mL IL-2 was added to each well. TILs were harvested after 10 days of REP.

### Results

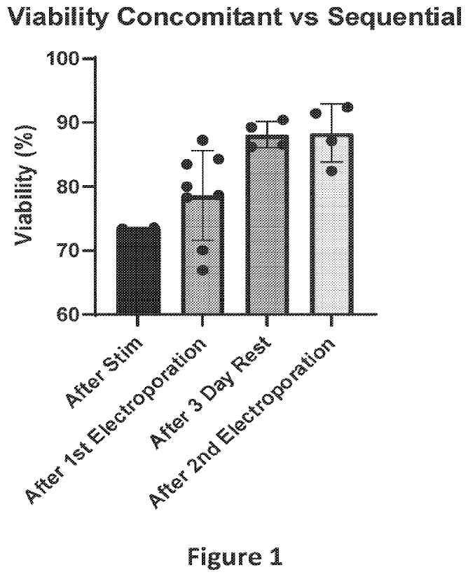

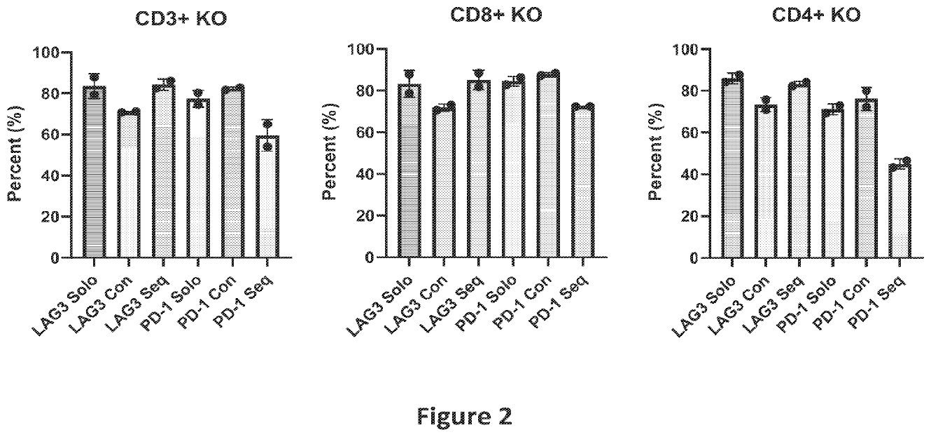

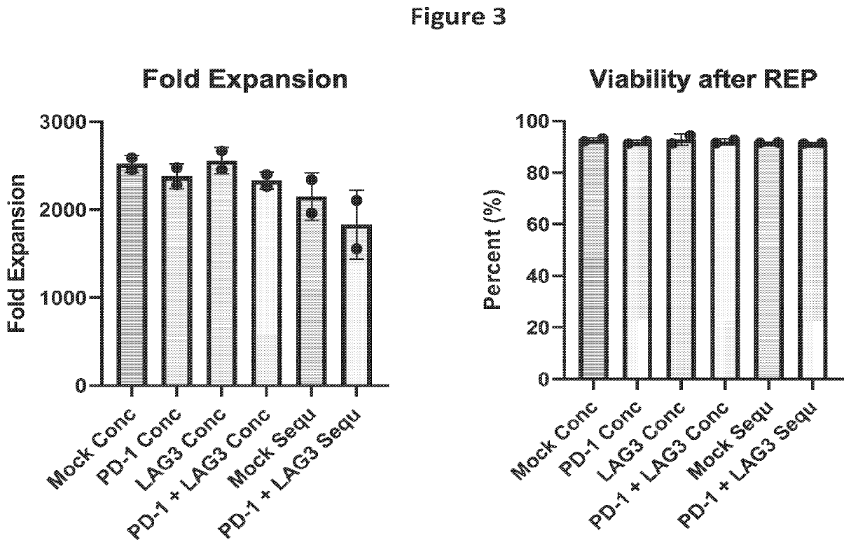

FIG. 1 shows the viability of TILs after sequential electroporation. FIG. 2 shows LAG3 and PD-1 KO efficiency in CD3+ (FIG. 2A), CD8+ (FIG. 2B) and CD4+ (FIG. 2C) TILs. The expression of KO target was measured by flow cytometry after stimulating the cells overnight with aCD3/aCD28 beads (1:5 ratio, beads: cells). KO efficiency was calculated by subtracting the expression of KO target in TALEN cells from the expression of KO target in Mock cells (cells that underwent sham electroporation, in which no RNA is present) divided by the expression of KO target in Mock cells. ((Mock-TALEN)/(Mock))*100=% KO Efficiency. FIG. 3 shows fold expansion (FIG. 3A) and viability (FIG. 3B) of concomitantly and sequentially electroporated TILs after REP.

### Example 3: Stimulation Day Testing

### Tumor Preparation

Tumor samples freshly resected from patients having two different cancers (head & neck and breast) were fragmented into approximately 2-6-mm³ fragments.

Tumor Processing. Obtained tumor specimen and transferred into suite at 2-8° C. immediately for processing. Aliquoted tumor wash media. Tumor wash 1 was performed using 8″ forceps (W3009771). The tumor was removed from the specimen bottle and transferred to the “Wash 1” dish prepared. This was followed by tumor wash 2 and tumor wash 3. Measured and assessed tumor. Assessed whether >30% of entire tumor area observed to be necrotic and/or fatty tissue. Cleaned up dissection if applicable. If tumor was large and >30% of tissue exterior was observed to be necrotic/fatty, performed “clean up dissection” by removing necrotic/fatty tissue while preserving tumor inner structure using a combination of scalpel and/or forceps. Dissected tumor. Using a combination of scalpel and/or forceps, cut the tumor specimen into even, appropriately sized fragments (up to 6 intermediate fragments). Transferred intermediate tumor fragments. Dissected tumor fragments into pieces approximately 3×3×3 mm in size. Stored Intermediate fragments to prevent drying. Repeated intermediate fragment dissection. Determined number of pieces collected. If desirable tissue remained, selected additional favorable tumor pieces from the “favorable intermediate fragments” 6-well plate to fill the drops for a maximum of 50 pieces.

### Production of PreREP TIL Product

### Day 0

CM1 Media Preparation. In a biological safety cabinet (BSC) added reagents to RPMI 1640 Media bottle. Added per bottle: Heat Inactivated Human AB Serum (100.0 mL); GlutaMax™ (10.0 mL); Gentamicin sulfate, 50 mg/ml (1.0 mL); 2-mercaptoethanol (1.0 mL).

Remove unnecessary materials from BSC. Passed out media reagents from BSC, left Gentamicin Sulfate and HBSS in BSC for Formulated Wash Media preparation.

Thaw IL-2 aliquot. Thawed one 1.1 mL IL-2 aliquot (6×10⁶ IU/mL) (BR71424) until all ice melted. Recorded IL-2: Lot # and Expiry.

Transfer IL-2 stock solution to media. In the BSC, transferred 1.0 mL of IL-2 stock solution to the CM1 Day 0 Media Bottle prepared. Added CM1 Day 0 Media 1 bottle and IL-2 (6×10⁶ IU/ml) 1.0 mL.

Pass G-REX100MCS into BSC. Aseptically passed G-REX100MCS (W3013130) into the BSC.

Pumped all Complete CM1 Day 0 Media into G-REX100MCS flask. Tissue Fragments Conical or GRex100MCS.

Tumor Wash Media Preparation. In the BSC, added 5.0 ml Gentamicin (W3009832 or W3012735) to 1×500 mL HBSS Media (W3013128) bottle. Added per bottle: HBSS (500.0 mL); Gentamicin sulfate, 50 mg/ml (5.0 mL). Filtered HBSS containing gentamicin prepared through a 1 L 0.22-micron filter unit (W1218810).

Prepare conical tube. Transferred tumor pieces to a 50 ml conical tube. Prepared BSC for G-REX100MCS. Removed G-REX100MCS from incubator. Aseptically passed G-REX100MCS flask into the BSC. Added tumor fragments to G-REX100MCS flask. Evenly distributed pieces.

Incubate G-REX100MCS at the following parameters: Incubated G-REX flask: Temperature LED Display: 37.0±2.0° C.; CO₂ Percentage: 5.0±1.5% CO₂.

The pre-REP step was performed by culturing≤50 tumor fragments in a G-REX-100MCS flask in the presence of CM1 with 6000 IU/mL IL-2 for 6-9-days.

After process was complete, discarded any remaining warmed media and thawed aliquots of IL-2.

TIL Harvest. Preprocessing table. Incubator parameters: Temperature LED display: 37.0±2.0° C.; CO₂ Percentage: 5.0±1.5% CO₂. Removed G-REX100MCS from incubator. Prepared 300 mL Transfer Pack. Weld transferred packs to G-REX100MCS.

Prepare flask for TIL Harvest and initiation of TIL Harvest. Using the GatheRex, transferred the cell suspension through the blood filter into the 300 mL transfer pack. Inspected membrane for adherent cells.

Rinse flask membrane. Closed clamps on G-REX100MCS. Ensured all clamps were closed. Heat sealed the TIL and the “Supernatant” transfer pack. Calculated volume of TIL suspension. Prepared Supernatant Transfer Pack for Sampling.

Incubate TIL. Placed TIL transfer pack in incubator until needed. Performed cell counts and calculations. Determined the Average of Viable Cell Concentration and Viability of the cell counts performed. Viability÷2. Viable Cell Concentration÷2. Determined Upper and Lower Limit for counts. Lower Limit: Average of Viable Cell Concentration×0.9. Upper Limit: Average of Viable Cell Concentration×1.1. Confirmed both counts within acceptable limits. Determined an average Viable Cell Concentration from all four counts performed.

The procedures for obtaining cell and viability counts used the Nexcelom Cellometer K2 or equivalent cell counter.

Adjust Volume of TIL Suspension: Calculated the adjusted volume of TIL suspension after removal of cell count samples. Total TIL Cell Volume (A). Volume of Cell Count Sample Removed (4.0 mL) (B) Adjusted Total TIL Cell Volume C=A−B.

Calculate Total Viable TIL Cells. Average Viable Cell Concentration*: Total Volume; Total Viable Cells: C=A×B.

### Electroporation of PreREP TIL Product

PreREP TIL products were thawed and resuspended at 1e6/mL in CM1 with 6000 IU/mL IL-2. 3e6 TILs were plated in a GREX 24-well plate.

TILs were activated on different days (Day 0, 3, 5, 7) with GMP TransAct™ (Miltenyi Biotec) at 1:17.5 dilution. The plate was incubated at 37° C. until day 9. The activated TILs were combined to more than 14e6 live cells, span down, and resuspended at 50e6/mL in Electroporation Medium T.

PD-1 TALEN mRNA was prepared according to the following conditions:

| PD-1 TALEN | | 4 ug/2e6 cells |
|---|---|---|
| 49186 | 2 ug/uL | 2 uL |
| 49187 | 2 ug/uL | 2 uL |

A 24 well plate was prepared with 2 mL of CM1+1000 IU/mL IL-2 in each well and kept at 30° C. at least 30 minutes before starting of electroporation. BTX Electroporator was set up to run the following protocol:

| | Group 1 | Group 2 | Group 3 |
|---|---|---|---|
| Amplitude (in V) | 200 | 200 | 130 |
| Duration (in ms) | 0.1 | 0.1 | 0.2 |
| Internal (in ms) | 0.2 | 100 | 2 |
| Number | 1 | 1 | 4 |

TILs were electroporated on Day 9 with PD-1 TAL and rested overnight at 30° C. C and then at 37° C. for 2 days (total 3 day rest time) in CM1 with 1,000 IU/mL IL-2, then electroporated again with PD-1 TAL. For the second electroporation, PD-1 TAL mRNA was prepared according to the following conditions:

| PD-1 TALEN | | 2 ug/1e6 cells |
|---|---|---|
| 49186 | 2 ug/uL | 1 uL |
| 49187 | 2 ug/uL | 1 uL |

After electroporation, the cells were incubated overnight at 30° C. in CM1 with 1,000 IU/mL IL-2. The concomitantly or sequentially electroporated TILs were incubated overnight at 30 C in CM1 with 1,000 IU/mL IL-2, then went through a rapid expansion process (REP) using 3 mL of CM2, 3000 IU/mL IL-2, 30 ng/ml OKT3, and 30e6 feeders per well of a GREX 24-well plate.

Feeder Cell Preparation. Gamma-irradiated peripheral mononuclear cells (PBMCs) are required for REP of TILs. Feeder cells were prepared from the leukapheresis of whole blood taken from individual donors. The leukapheresis product was subjected to centrifugation over Ficoll-Hypaque, washed, irradiated, and cryopreserved under GMP conditions.

On Day 5 of the REP, 5 mL of CM4+3000 IU/mL IL-2 was added to each well. TILs were harvested after 10 days of REP.

### Results

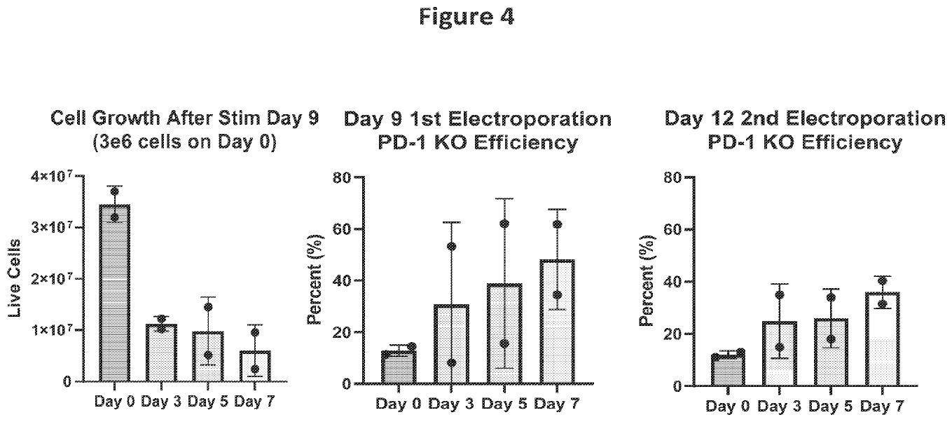

FIGS. 4A-4C show cell growth after stimulation at different days (FIG. 4A), 1st electroporation PD-1 KO efficiency (FIG. 4B), and 2nd electroporation PD-1 KO efficiency (FIG. 4C). The expression of KO target was measured by flow cytometry after stimulating the cells overnight with aCD3/aCD28 beads (1:5 ratio, beads: cells). KO efficiency was calculated by subtracting the expression of KO target in TALEN cells from the expression of KO target in Mock cells (cells that underwent sham electroporation, in which no RNA is present) divided by the expression of KO target in Mock cells. ((Mock-TALEN)/(Mock))*100=% KO Efficiency.

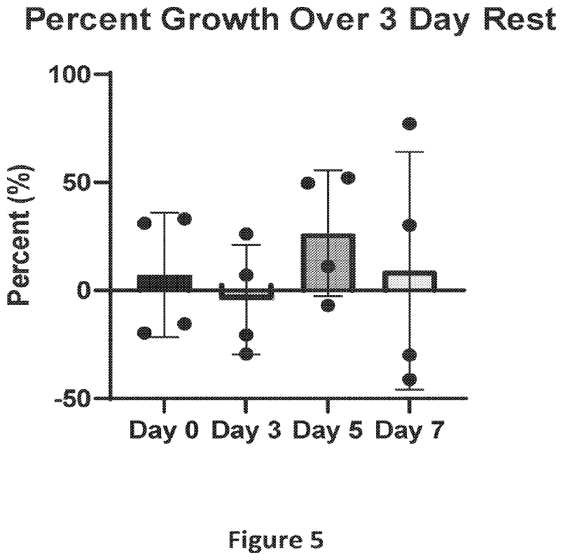

FIG. 5 shows percentage of TIL growth over 3-day rest period with stimulation on different days (Day 0, 3, 5, 7).

### Example 4: Sequencing of PD1 and TIGIT TALEN Electroporation

### Methods

Pre-REP TIL lines (N=2) from different indications (head & neck and breast) were thawed and resuspended in 6.6 mL of CM1 with 3000 IU/mL IL-2, and plated in a GREX 24-well plate.

TILs were activated on day 0 and day 2 with GMP TransAct at a 1:17.5 dilution to simulate a 2-day or 4-day activation period prior to first electroporation. The plate was incubated at 37° C. until day 4.

TIGIT and PD-1 TALEN mRNA was prepared according to the following conditions:

| TIGIT TALEN | | 3 ug/2e6 cells |
|---|---|---|
| 39233 | 2 ug/uL | 1.5 uL |
| 39234 | 2 ug/uL | 1.5 uL |
| PD-1 TALEN | | 4 ug/2e6 cells |
| 49186 | 2 ug/uL | 2 uL |
| 49187 | 2 ug/uL | 2 uL |

A 24 well plate was prepared with 2 mL of CM1+1000 IU/mL IL-2 in each well and kept at 30° C. at least 30 minutes before starting of electroporation. BTX Electroporator was set up to run the following protocol:

| | Group 1 | Group 2 | Group 3 |
|---|---|---|---|
| Amplitude (in V) | 200 | 200 | 130 |
| Duration (in ms) | 0.1 | 0.1 | 0.2 |
| Internal (in ms) | 0.2 | 100 | 2 |
| Number | 1 | 1 | 4 |

The cells were electroporated on Day 4 with PD-1 or TIGIT TAL and rested overnight at 30° C. C and then at 37° C. for 2 days (total 3 day rest time) in CM1 with 1,000 IU/mL IL-2, then electroporated again with TIGIT or PD-1 TAL to determine the sequencing of electroporation and stimulation time to maximize both PD-1 and TIGIT KO efficiency.

After electroporation, the cells were incubated overnight at 30° C. in CM1 with 1,000 IU/mL IL-2. The concomitantly or sequentially electroporated TILs were incubated overnight at 30 C in CM1 with 1,000 IU/mL IL-2, then REP′d using 3 ml of CM2, 3000 IU/mL IL-2, 30 ng/ml OKT3, and 30e6 feeders per well of a GREX 24-well plate. On Day 5 of the REP, 5 mL of CM4+3000 IU/mL IL-2 was added to each well. TILs were harvested after 10 days of REP.

### Results

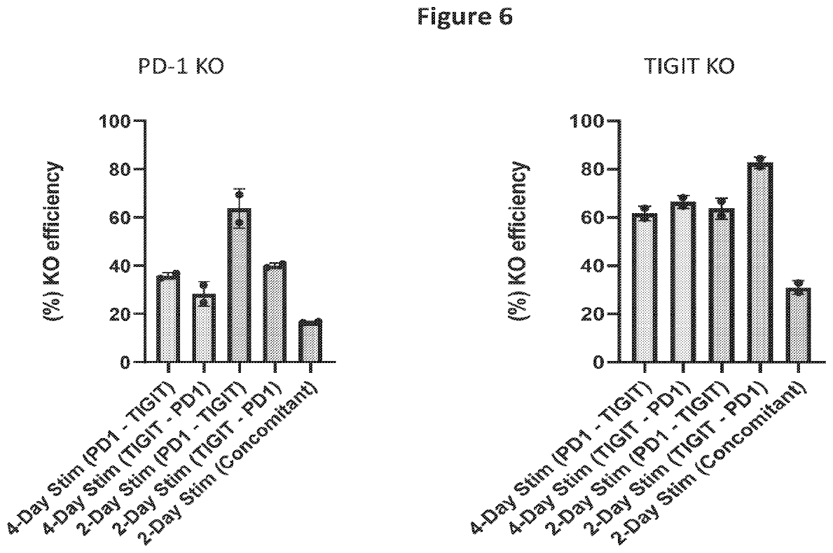

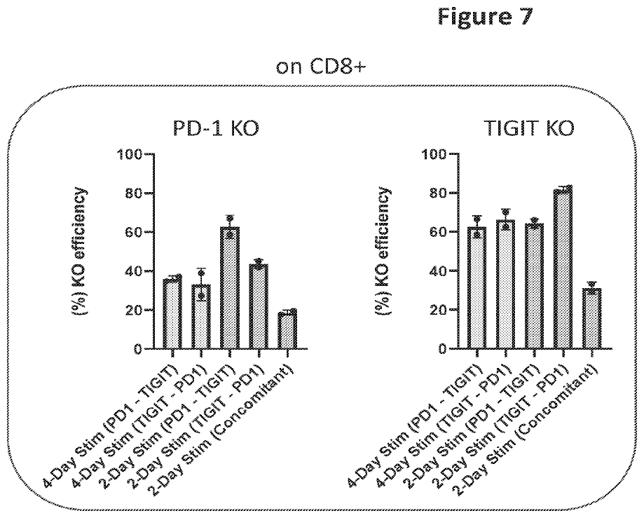

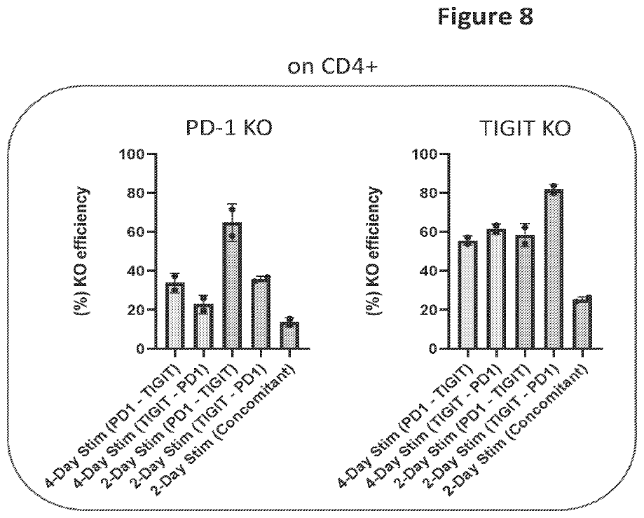

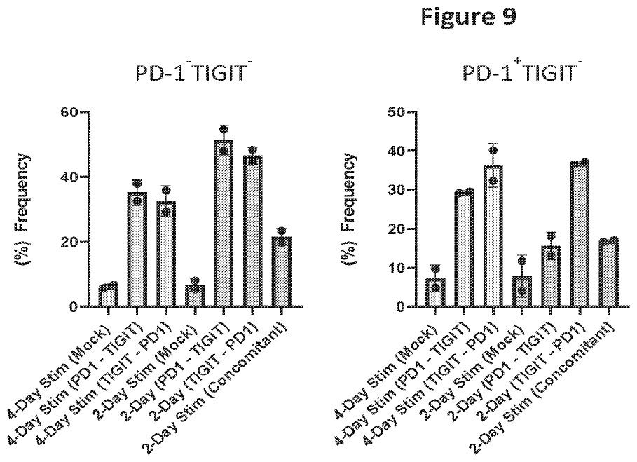

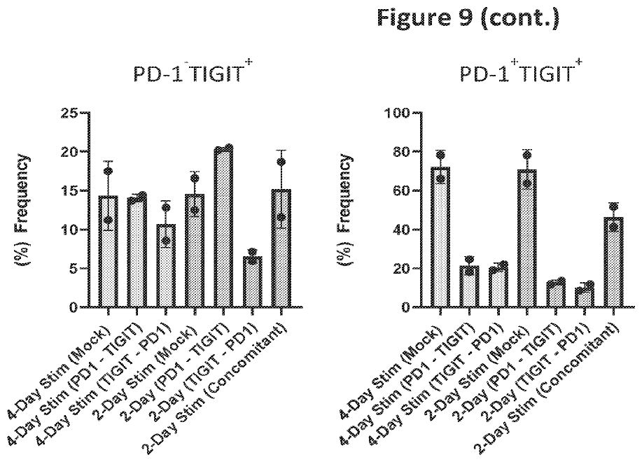

FIGS. 6A and 6B show PD-1 and TIGIT KO efficiencies on total CD3+ TILs with 4-day and 2-day stimulation. FIGS. 7A and 7B show PD-1 and TIGIT KO efficiencies on total CD8+ TILs with 4-day and 2-day stimulation. FIGS. 8A and 8B show PD-1 and TIGIT KO efficiencies on total CD4+ TILs with 4-day and 2-day stimulation. FIGS. 9A-9D show frequency of PD-1 and TIGIT expression on CD3+ TILs. The expression of KO target was measured by flow cytometry after stimulating the cells overnight with aCD3/aCD28 beads (1:5 ratio, beads: cells). KO efficiency was calculated by subtracting the expression of KO target in TALEN cells from the expression of KO target in Mock cells (cells that underwent sham electroporation, in which no RNA is present) divided by the expression of KO target in Mock cells. ((Mock-TALEN)/(Mock))*100=% KO Efficiency.

### Example 5: Titration for PD-1 and TIGIT TALEN mRNA

### Methods

Tumor samples from different indications (renal, lung and melanoma) were cut into 3 mm fragments and incubated at 37° C. in CM1 with 6000 IU/ml IL-2. On day 7, TransAct at a 1:17.5 dilution was added to the TILs to initiate the stimulation process.

On day 9, 5e6 TILs were resuspend in 250 μl Thermo electroporation buffer and electroporated with PD-1 or TIGIT TALEN mRNA at a concentration of 1 μg/1e6 cells, 2 μg/1e6 cells, 3 ug/1e6 cells, 4 μg/1e6 cells, or 8 μg/1e6 cells. TILs were electroporated using Neon (ThermoFisher) at 2300 V, 2 ms, 3 pulses. On day 12, after a 3 day resting period, TILs were electroporated with PD-1 or TIGIT TALEN mRNA again, followed by an overnight resting period.

On day 13, 2e5 TILs were REP′d using 10 mL of CM2, 3000 IU/mL IL-2, 30 ng/ml OKT3, and 10e6 iPBMCs. On Day 5 of the REP, 80 mL of CM4 was added to each well. TILs were harvested after 9 or 11 days of REP (on day 22 or day 24).

### Results

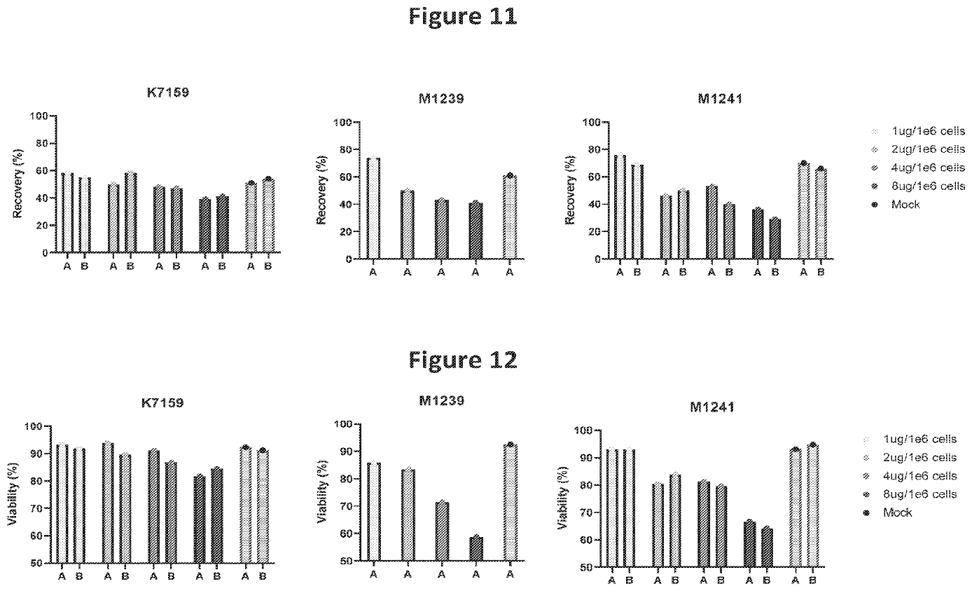

FIGS. 11A-11C show cell recovery after electroporation of different concentrations of PD-1 TALEN mRNA and 3 days of resting. FIGS. 12A-12C show cell viability after electroporation of different concentrations of PD-1 TALEN mRNA and 3 days of resting. A: D22 harvesting; B: D24 harvesting.

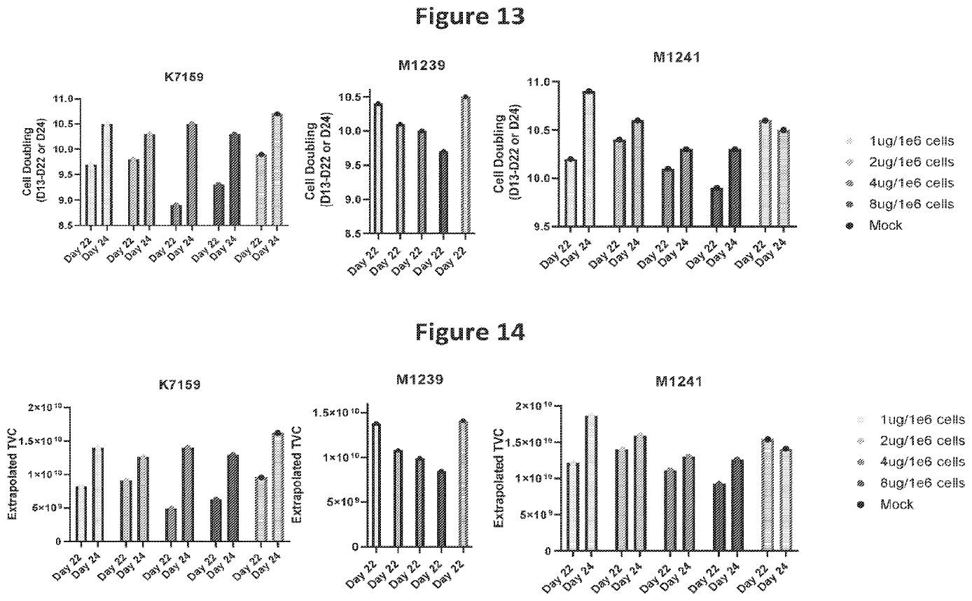

FIGS. 13A-13C show cell doubling numbers during REP (D22 and D24) after electroporation of different concentrations of PD-1 TALEN mRNA and 3 days of resting. FIGS. 14A-14C show extrapolated total viable cells during REP (D22 and D24) after electroporation of different concentrations of PD-1 TALEN mRNA and 3 days of resting.

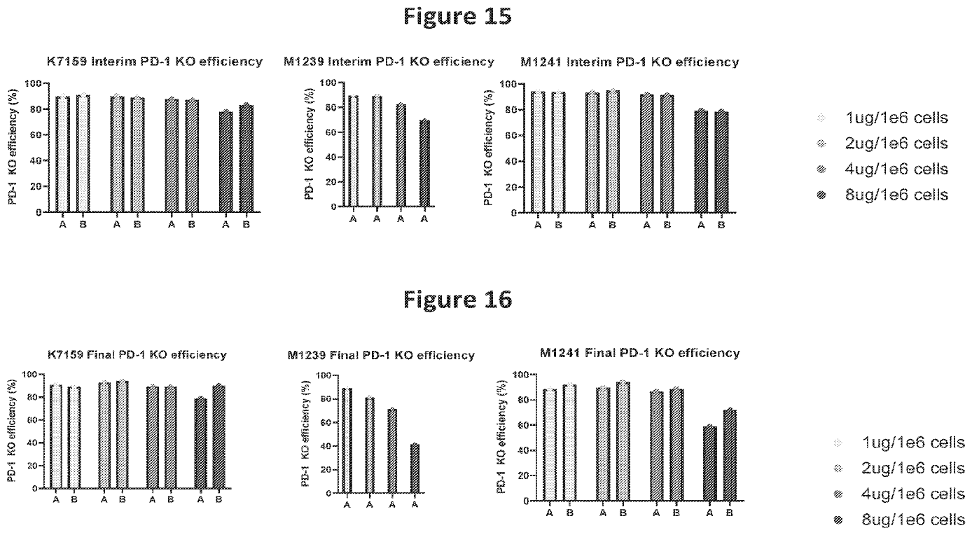

FIGS. 15A-15C show interim PD-1 KO efficiency after electroporation of different concentrations of PD-1 TALEN mRNA and 3 days of resting. FIGS. 16A-16C show final PD-1 KO efficiency after electroporation of different concentrations of PD-1 TALEN mRNA and 3 days of resting. A: D22 harvesting; B: D24 harvesting. The expression of KO target was measured by flow cytometry after stimulating the cells overnight with aCD3/aCD28 beads (1:5 ratio, beads: cells). KO efficiency was calculated by subtracting the expression of KO target in TALEN cells from the expression of KO target in Mock cells (cells that underwent sham electroporation, in which no RNA is present) divided by the expression of KO target in Mock cells. ((Mock-TALEN)/(Mock))*100=% KO Efficiency.

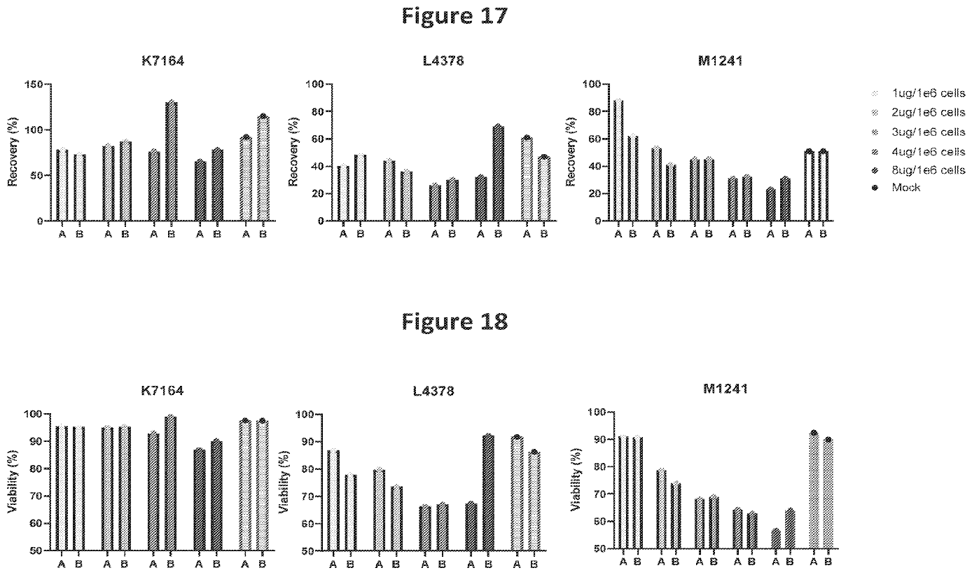

FIGS. 17A-17C show cell recovery after electroporation of different concentrations of TIGIT TALEN mRNA and 3 days of resting. FIGS. 18A-18C show cell viability after electroporation of different concentrations of TIGIT TALEN mRNA and 3 days of resting. A: D22 harvesting; B: D24 harvesting.

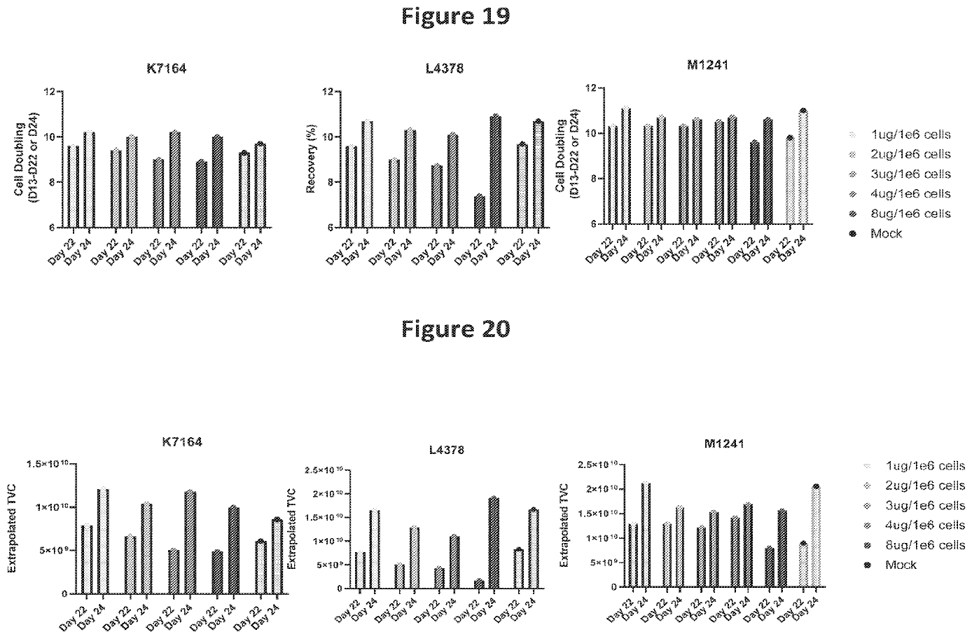

FIGS. 19A-19C show cell doubling numbers during REP (D22 and D24) after electroporation of different concentrations of TIGIT TALEN mRNA and 3 days of resting. FIGS. 20A-20C show extrapolated total viable cells during REP (D22 and D24) after electroporation of different concentrations of TIGIT TALEN mRNA and 3 days of resting.

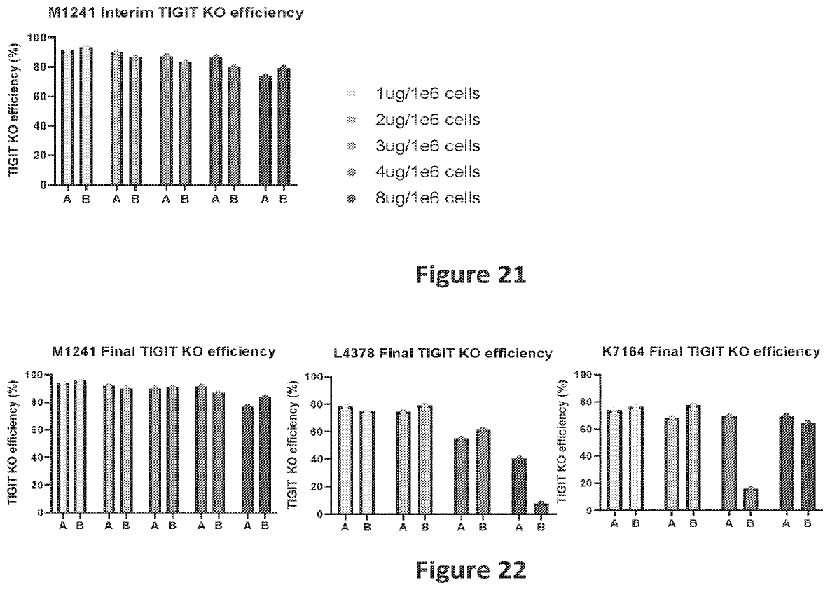

FIG. 21 shows interim TIGIT KO efficiency after electroporation of different concentrations of TIGIT TALEN mRNA and 3 days of resting. FIGS. 22A-22C show final TIGIT KO efficiency after electroporation of different concentrations of TIGIT TALEN mRNA and 3 days of resting. A: D22 harvesting; B: D24 harvesting. The expression of KO target was measured by flow cytometry after stimulating the cells overnight with aCD3/aCD28 beads (1:5 ratio, beads: cells). KO efficiency was calculated by subtracting the expression of KO target in TALEN cells from the expression of KO target in Mock cells (cells that underwent sham electroporation, in which no RNA is present) divided by the expression of KO target in Mock cells. ((Mock-TALEN)/(Mock))*100=% KO Efficiency.

### Example 6: Scaled-Up Process for PD-1 TIGIT DKO TIL with ⅕th Mock Electroporation

### Methods

Tumor samples are received in HypoThemosol approximately 24 to 96 hours after resection. After fragmentation, a small bioburden sample is removed in Transport medium, while the remaining fragments are transferred to a G-Rex100MCS with 1 L CM1 and 6000 IU/mL IL-2.

On day 7, the volume is reduced to approximately 100 mL, followed by addition of TransAct directly to the G-Rex100MCS.

On day 9, the volume is again reduced for electroporation using the Neon Electroporator (ThermoFisher) at 2300 V, 2 ms, 3 pulses. TILs are first divided into two groups, with one group comprising approximately 80% of the cultured cells (TALEN group). This group is electroporated with PD-1 TALEN mRNA at a concentration of 2 μg/1e6 cells. The second group of TILs (Mock group), comprising approximately 20% of the cultured cells, are subjected to a ‘sham’ electroporation, in which no RNA is present.

On day 12, after a 3 day resting period, TILs in the TALEN group are subjected to electroporation with TIGIT TALEN mRNA at a concentration of 2 μg/1e6 cells- and TILs in the Mock group are subjected to sham electroporation, followed by an overnight resting period.

On day 13, 4-10e6 TILs of the TALEN group and the Mock group are seeded in a G-REX100MCS flask with 1 L of CM2, 3000 IU/mL IL-2, 30 ng/mL OKT3, and 10e9 iPBMCs per flask.

On day 18 the total 1 L sample is transferred from a G-Rex100MCS to a G-Rex500MCS. Total volume is brought up to 5 L with CM4. Cells are harvested on Day 22 or Day 24.

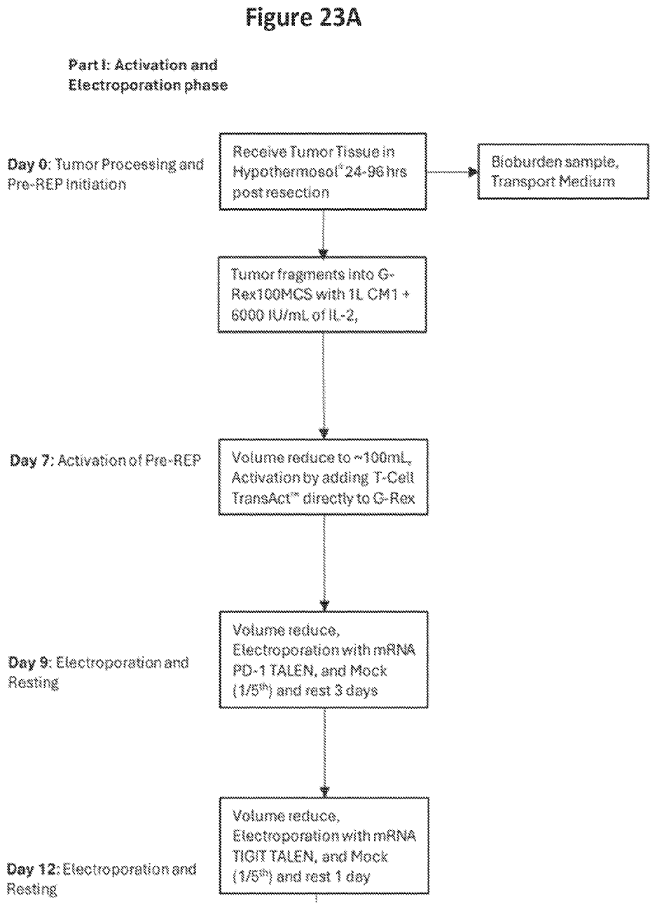

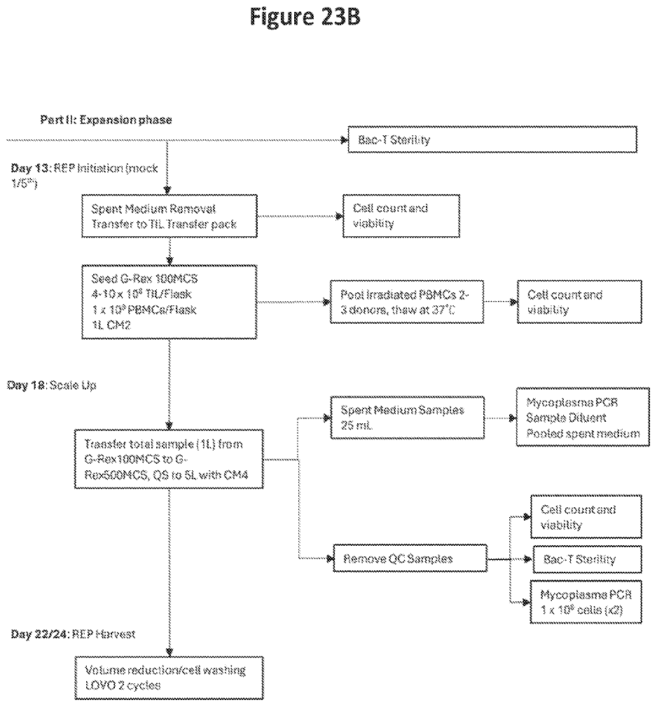

FIGS. 23A and 23B show an exemplary process flow of this scaled-up expansion method.

### Example 7: M1152 PDX TALEN Mouse Study

40 NOG hIL-2 female mice were randomized and injected with 1e6 cells of IOVM053119 (melanoma cell line derived from patient-passage 4). On Day 7 tumor measurements were initiated 2×/week with 3-5 days in between measurements. Adoptive transfer was performed on Day 19 when most tumors reached a size of 20-30 mm². Cells were resuspended at 50e6/mL in PBS for injection. Mice were randomized into groups with groups A-C receiving 10e6 TILs.

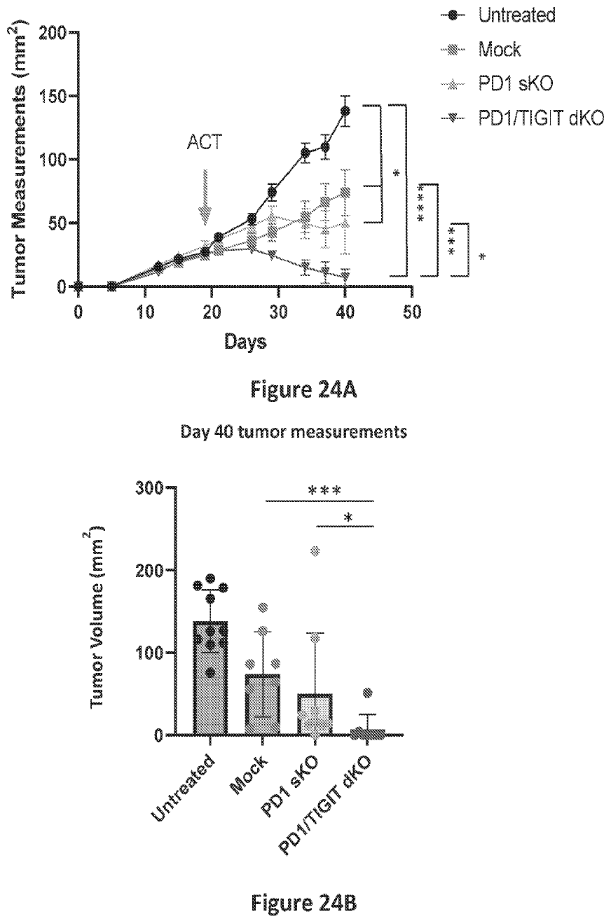

FIGS. 24A and 24B show that adoptive transfer of PD1/TIGIT dKO TIL led to increased tumor control compared to PD1 sKO TIL and mock control TIL.

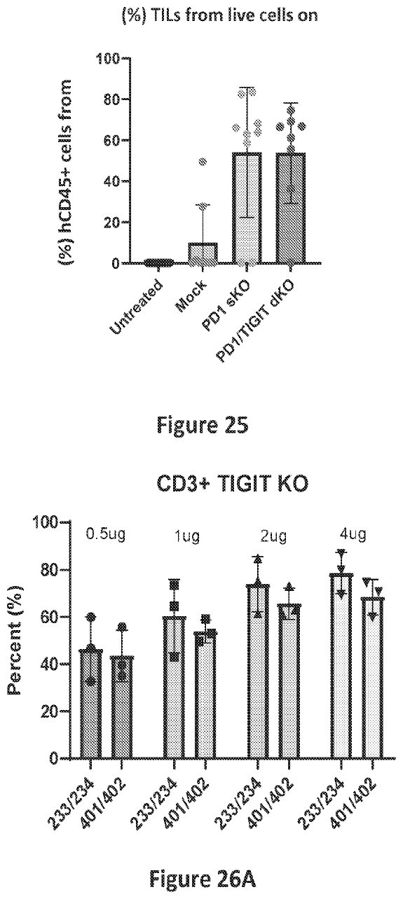

FIG. 25 shows similar recovery of TIL 21 days post adoptive transfer between PD1 sKO and PD1/TIGIT dKO TILs.

### Example 8: TIGIT mRNA Titration

Pre-REP TIL lines (N=3) from different indications (head & neck, lung, and breast) were thawed, activated (OKT3 platebound 300 ng/ml), and electroporated with 2 pairs of TIGIT mRNA TALEN at varying concentrations (0.5 ug, 1 μg, 2 ug, 4 ug per 1e6 cells). After electroporation, the cells were incubated overnight at 30 C in CM1 with 1,000 IU/mL IL-2 then REP′d.

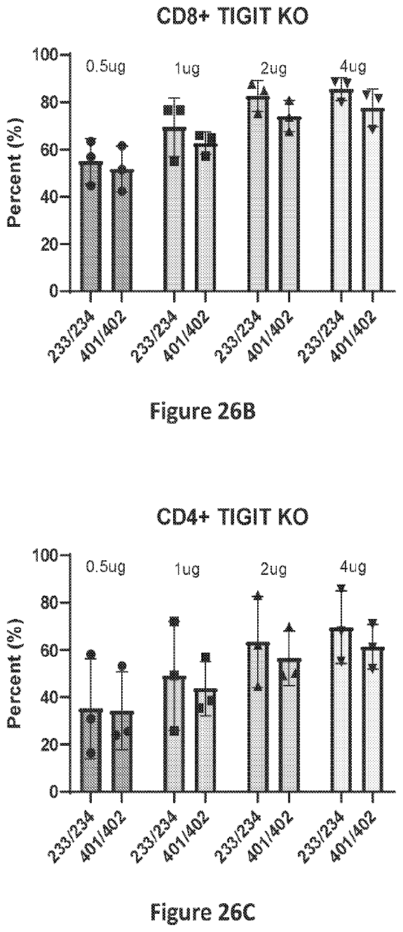

FIGS. 26A-26C show that strongest KO efficiency observed for 39233/39234 TALEN pair, but overall strong KO efficiency observed for both TALEN mRNA pairs at concentrations of 2-4 ug/million cells.

Drop in TIL viability was observed post electroporation with higher TALEN mRNA concentration (from over 75% in Mock electroporation to less than 70% in 4 μg/million cells).

### Example 9: PD-1 and TIGIT mRNA Titration

Five frozen pre-REP TIL lines (L4376, K7098, K7159, B5031, and M1243) were thawed, activated for 2 or 5 days, and electroporated with PD-1 or TIGIT mRNA TALEN at varying concentrations (0.25 μg, 0.5 ug, 1 μg, 2 ug per 1e6 cells). After electroporation, the cells were rested for 1 day and then REP′d for 9 (harvested on Day 22) or 11 days (harvested on Day 24). After harvest, the TILs were measured for recovery and viability post EP, harvest TVC and cell doublings. KO efficiency was assessed by flow cytometry and droplet digital PCR (ddPCR).

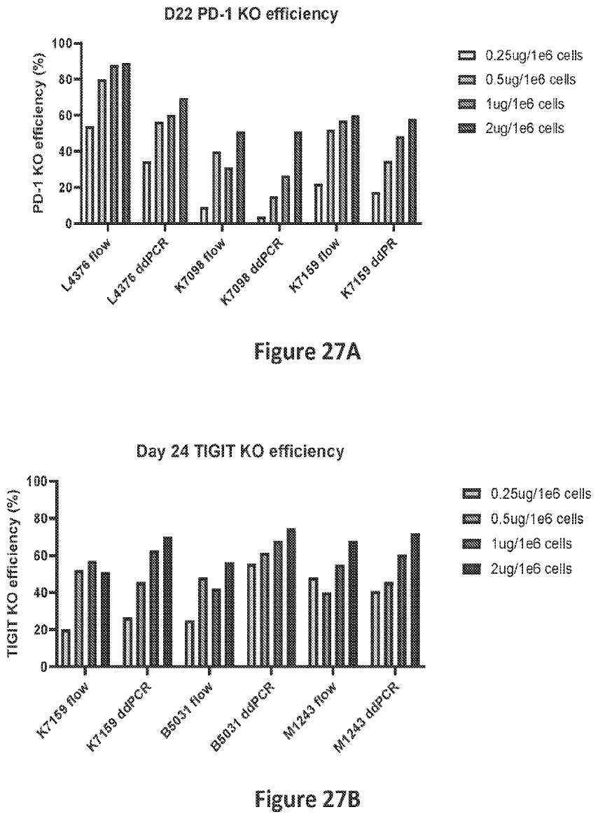

FIGS. 27A-27B show that PD-1 and TIGIT KO efficiency plateaued from 1 to 2 μg/1e6 cells.

Recovery, viability, harvest TVC and cell doublings were similar across all mRNA concentrations.

### Example 10: Small Scale Optimization for PD-1/TIGIT dKO TIL Generation

Four tumor specimens (M1248, EP11276, EP11277, T6086) (1 melanoma, 2 ER/PR+, 1 TNBC) were used for this study.

On Day 0, pre-REP was initiated by processing recently resected patient tumor specimen into 3 mm sized fragments and culturing in CM1. On Day 7, TILs were activated for 2 days by adding MACS® GMP T cell TransAct to the tumor fragment cell culture. On Day 9, TILs were either electroporated with PD-1 mRNA TALEN at 2 ug per 1e6 cells or subjected to sham electroporation, and rested for 3 days. On Day 12, TILs were either electroporated with TIGIT mRNA TALEN at 2 ug per 1e6 cells or subjected to sham electroporation. On Day 13, the TILs were REP′d by co-culturing the cells with irradiated PBMCs and anti-CD3 in CM2. On Day 18, CM4 was added for scale up. TILs were harvested on Day 22 or Day 24 and cryopreserved in CS10.

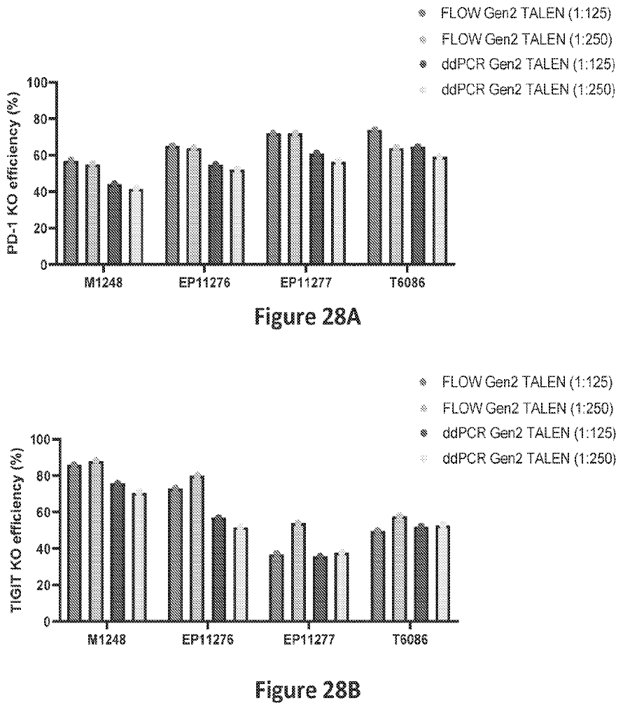

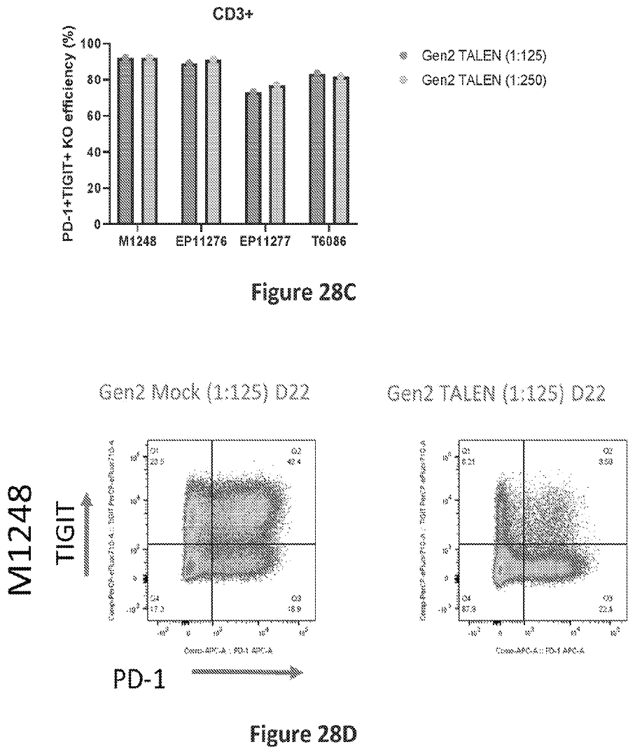

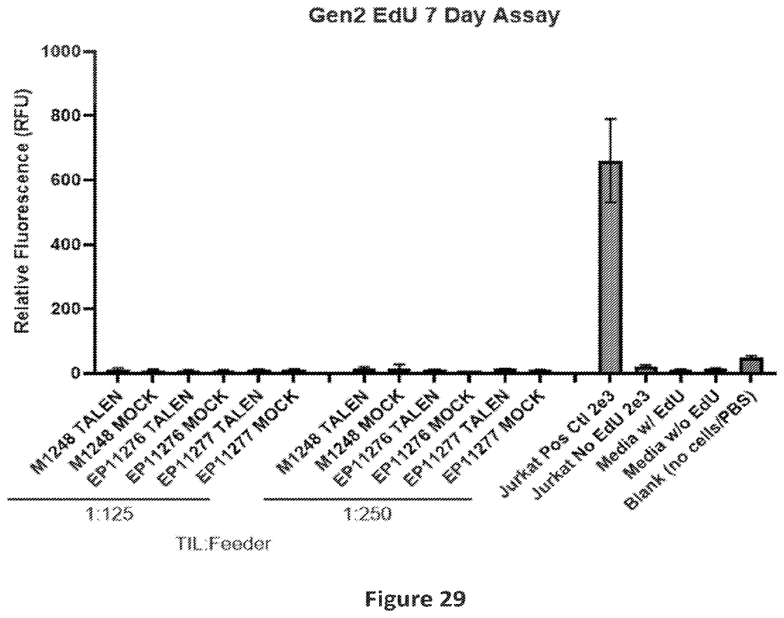

In process testing parameters included recovery and viability post-electroporation harvest TVC and cell doublings. KO efficiency was assessed by flow cytometry and ddPCR using cryopreserved Day 22 and D24 harvested samples (Table 17; FIG. 28). IL-2 independent proliferation assay for PD-1/TIGIT double KO TILs showed no proliferation (FIG. 29).

TABLE 15
Summary data of small scale PreREP, Activation and Electroporation.

| | M1248 | | EP11276 | | EP11277 | | T6086 | |
|---|---|---|---|---|---|---|---|---|
| | Gen2 | | Gen2 | | Gen2 | | Gen2 | |
| Parameter | TALEN | MOCK | TALEN | MOCK | TALEN | MOCK | TALEN | MOCK |
| Day 0 PreREP Fragments | 21 | | 17 | | 11 | | 47 | |
| Day 9 Post Act TVC (×10⁶) | 12.1 | | 5.50 | | 9.49 | | 308 | |
| Day 9 Pre-EP TVC (×10⁶) | 6.06 | 6.06 | 2.75 | 2.75 | 4.74 | 4.74 | 50.0 | 10.0 |
| Day 12 TVC Post-1st EP(×10⁶) | 3.00 | 3.00 | 4.61 | 5.67 | 3.06 | 2.90 | 51.3 | 11.0 |
| Day 12 Cell Recovery (3-day | 50 | 50 | 168 | 206 | 65 | 61 | 103 | 110 |
| rest post-1st EP) (%) | | | | | | | | |
| Day 13 Post-2nd EP TVC (×10⁶) | 1.63 | 1.9 | 3.08 | 4.50 | 2.12 | 2.27 | 32.2 | 7.3 |
| Day 13 Cell Recovery (%) | 54 | 63 | 67 | 79 | 69 | 78 | 62 | 67 |
| Day 13 REP initiation TVC | 0.4 | 0.4 | 0.4 | 0.4 | 0.4 | 0.4 | 0.4 | 0.4 |
| (×10⁶) | | | | | | | | |

TABLE 16
Summary data from Post-REP (A = 1:125 TIL:iPBMC; B = 1:250 TIL:iPBMC).

| | M1248 | | | | EP11276 | | | | EP11277 | | | | T6086 | | | |
|---|---|---|---|---|---|---|---|---|---|---|---|---|---|---|---|---|
| | Gen2 | | | | Gen2 | | | | Gen2 | | | | Gen2 | | | |
| | TALEN | | MOCK | | TALEN | | MOCK | | TALEN | | MOCK | | TALEN | | MOCK | |
| Parameters | A | B | A | B | A | B | A | B | A | B | A | B | A | B | A | B |
| Day 13 REP TVC | 4.0 | 4.0 | 4.0 | 4.0 | 4.0 | 4.0 | 4.0 | 4.0 | 4.0 | 4.0 | 4.0 | 4.0 | 4.0 | 4.0 | 4.0 | 4.0 |
| (×10⁵) | | | | | | | | | | | | | | | | |
| Day 22 REP TVC | 2.0 | 2.4 | 2.6 | 3.0 | 3.1 | 3.8 | 3.3 | 3.5 | 1.9 | 2.3 | 1.9 | 2.3 | 1.7 | 2.0 | 2.3 | 2.4 |
| (×10⁸) | | | | | | | | | | | | | | | | |
| Day 24 REP TVC | 3.1 | NA | 2.7 | NA | 3.7 | NA | 4.4 | NA | 2.5 | NA | 2.9 | NA | 2.5 | NA | 2.7 | NA |
| (×10⁸) | | | | | | | | | | | | | | | | |
| Doublings (D13- | 9.0 | 9.2 | 9.3 | 9.5 | 9.6 | 9.9 | 9.7 | 9.8 | 8.9 | 9.2 | 8.9 | 9.2 | 8.8 | 9.0 | 9.2 | 9.2 |
| D22) | | | | | | | | | | | | | | | | |
| Doublings (D13- | 9.6 | NA | 9.4 | NA | 9.9 | NA | 10 | NA | 9.3 | NA | 9.5 | NA | 9.3 | NA | 9.4 | NA |
| D24) | | | | | | | | | | | | | | | | |
| D22 Extrapolated | 8 | 10 | 10 | 12 | 12 | 16 | 14 | 14 | 8 | 10 | 8 | 10 | 7 | 8 | 9 | 10 |
| TVC (×10⁹)² | | | | | | | | | | | | | | | | |
| D24 Extrapolated | 12 | NA | 10 | NA | 15 | NA | 18 | NA | 10 | NA | 12 | NA | 9 | NA | 11 | NA |
| TVC (×10⁹)² | | | | | | | | | | | | | | | | |

TABLE 17
Summary data of PD-1/TIGIT KO Efficiency (A = 1:125 TIL:iPBMC; B = 1:250 TIL:iPBMC).

| | M1248 | | | | EP11276 | | | | EP11277 | | | | T6086 | | | |
|---|---|---|---|---|---|---|---|---|---|---|---|---|---|---|---|---|
| | Gen2 | | | | Gen2 | | | | Gen2 | | | | Gen2 | | | |
| | TALEN | | MOCK | | TALEN | | MOCK | | TALEN | | MOCK | | TALEN | | MOCK | |
| Parameters | A | B | A | B | A | B | A | B | A | B | A | B | A | B | A | B |
| D22 PD-1 KO | 57 | 55 | NA | NA | 65 | 64 | NA | NA | 72 | 73 | NA | NA | 74 | 64 | NA | NA |
| efficiency (%) | | | | | | | | | | | | | | | | |
| D24 PD-1 KO | —¹ | —¹ | NA | NA | —¹ | —¹ | NA | NA | —¹ | —¹ | NA | NA | —¹ | —¹ | NA | NA |
| efficiency (%) | | | | | | | | | | | | | | | | |
| D22 TIGIT KO | 86 | 88 | NA | NA | 73 | 80 | NA | NA | 37 | 54 | NA | NA | 50 | 58 | NA | NA |
| efficiency (%) | | | | | | | | | | | | | | | | |
| D24 TIGIT KO | —¹ | —¹ | NA | NA | —¹ | —¹ | NA | NA | —¹ | —¹ | NA | NA | —¹ | —¹ | NA | NA |
| efficiency (%) | | | | | | | | | | | | | | | | |
| D22 PD-1/TIGIT KO | 92 | 92 | NA | NA | 89 | 91 | NA | NA | 73 | 77 | NA | NA | 83 | 82 | NA | NA |
| efficiency (%) | | | | | | | | | | | | | | | | |
| D24 PD-1/TIGIT KO | —¹ | —¹ | NA | NA | —¹ | —¹ | NA | NA | —¹ | —¹ | NA | NA | —¹ | —¹ | NA | NA |
| efficiency (%) | | | | | | | | | | | | | | | | |

TABLE 18
Summary of IFNg release after Dyna Bead Stimulation (MTH-0041 and MTH-
0042) (A = 1:125 TIL:iPBMC; B = 1:250 TIL:iPBMC).

| | M1248 | | | | EP11276 | | | | EP11277 | | | | T6086 | | | |
|---|---|---|---|---|---|---|---|---|---|---|---|---|---|---|---|---|
| | Gen2 | | | | Gen2 | | | | Gen2 | | | | Gen2 | | | |
| | TALEN | | MOCK | | TALEN | | MOCK | | TALEN | | MOCK | | TALEN | | MOCK | |
| Parameters | A | B | A | B | A | B | A | B | A | B | A | B | A | B | A | B |
| D22 IFNg | 17257 | 13889 | NA | NA | 929 | 1118 | NA | NA | 4351 | 5296 | NA | NA | —¹ | —¹ | NA | NA |
| (pg/ml) | | | | | | | | | | | | | | | | |
| D24 IFNg | —¹ | —¹ | NA | NA | —¹ | —¹ | NA | NA | —¹ | —¹ | NA | NA | —¹ | —¹ | NA | NA |
| (pg/ml) | | | | | | | | | | | | | | | | |

TABLE 19
Phenotype: T cell content and Impurities determined using Lyric Tube-1 (A = 1:125 TIL:iPBMC; B = 1:250 TIL:iPBMC).

| | M1248 | | | | EP11276 | | | | EP11277 | | | | T6086 | | | |
|---|---|---|---|---|---|---|---|---|---|---|---|---|---|---|---|---|
| | Gen2 | | | | Gen2 | | | | Gen2 | | | | Gen2 | | | |
| Parameters | TALEN | | MOCK | | TALEN | | MOCK | | TALEN | | MOCK | | TALEN | | MOCK | |
| (% of Live) | A | B | A | B | A | B | A | B | A | B | A | B | A | B | A | B |
| T cells (CD#+/ | 95.6 | 94.9 | 96.1 | 97.6 | 99.3 | 99.3 | 99.2 | 89.9 | 97.1 | 96.9 | 90.6 | 96.9 | 97.5 | 96.5 | 97.5 | 98.2 |
| CD45+) (%) | | | | | | | | | | | | | | | | |
| TCR α/β+ T | 93.4 | 92.3 | 97.0 | 94.3 | 98.3 | 98.8 | 98.2 | 98.2 | 95.4 | 95.7 | 88.8 | 95.7 | 93.7 | 93.6 | 94.1 | 94.7 |
| cells (%) | | | | | | | | | | | | | | | | |
| NKT Cells | 20.9 | 26.7 | 21.6 | 25.6 | 8.6 | 7.9 | 8.6 | 7.1 | 8.2 | 8.9 | 8.2 | 7.6 | 12.3 | 9.2 | 13.2 | 9.5 |
| (%) | | | | | | | | | | | | | | | | |
| MCSP+ | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 |
| Tumor Cells | | | | | | | | | | | | | | | | |
| (%) | | | | | | | | | | | | | | | | |
| NK Cells (%) | 1.8 | 2.2 | 1.2 | 1.2 | 0.1 | 0.1 | 0.1 | 0.1 | 0.4 | 0.6 | 0.5 | 0.4 | 0.4 | 0.4 | 0.3 | 0.3 |
| B Cells (%) | 0.0 | 0.1 | 0.0 | 0.0 | 0.0 | 0.1 | 0.0 | 0.1 | 0.0 | 0.0 | 0.0 | 0.0 | 0.0 | 0.1 | 0.0 | 0.1 |
| Monocytes | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 |
| (%) | | | | | | | | | | | | | | | | |

TABLE 20
Phenotype: CD4/CD8 content, Memory and Exhaustion status determined using
Lyric Tube-2 (A = 1:125 TIL:iPBMC; B = 1:250 TIL:iPBMC).

| | M1248 | | | | EP11276 | | | | EP11277 | | | | T6086 | | | |
|---|---|---|---|---|---|---|---|---|---|---|---|---|---|---|---|---|
| Parameters | Gen2 | | | | Gen2 | | | | Gen2 | | | | Gen2 | | | |
| (% of Live | TALEN | | MOCK | | TALEN | | MOCK | | TALEN | | MOCK | | TALEN | | MOCK | |
| CD3+) | A | B | A | B | A | B | A | B | A | B | A | B | A | B | A | B |
| CD4+ T cells | 37.5 | 38.1 | 35.5 | 33.3 | 52.1 | 58.6 | 46.8 | 53.8 | 78.5 | 77.2 | 62.7 | 64.5 | 31.0 | 32.6 | 15.5 | 16.2 |
| (%) | | | | | | | | | | | | | | | | |
| CD8+ T cells | 50.7 | 50.0 | 54.7 | 54.8 | 41.6 | 35.3 | 48.3 | 40.7 | 15.8 | 16.7 | 26.2 | 25.0 | 59.3 | 55.9 | 75.6 | 74.6 |
| (%) | | | | | | | | | | | | | | | | |
| T_{EM} (%) | 84.6 | 80.8 | 91.6 | 92.4 | 90.7 | 89.6 | 90.8 | 89.3 | 76.1 | 78.2 | 85.2 | 85.4 | 73.3 | 82.8 | 88.0 | 83.5 |
| T_{CM} (%) | 14.1 | 16.9 | 7.7 | 6.9 | 8.1 | 9.1 | 6.5 | 9.3 | 23.1 | 20.6 | 14.4 | 14.1 | 26.0 | 16.5 | 11.6 | 16.1 |
| T_{EMRA} (%) | 0.8 | 1.1 | 0.3 | 0.3 | 1.0 | 0.8 | 2.3 | 0.8 | 0.5 | 0.6 | 0.3 | 0.3 | 0.2 | 0.3 | 0.2 | 0.1 |
| T_{N}/_{TSCM} (%) | 0.6 | 1.3 | 0.3 | 0.4 | 0.2 | 0.5 | 0.4 | 0.6 | 0.4 | 0.6 | 0.2 | 0.2 | 0.5 | 0.5 | 0.2 | 0.3 |
| LAG3+ T | 45.1 | 41.0 | 50.3 | 51.1 | 24.9 | 30.8 | 20.9 | 30.7 | 34.6 | 32.1 | 18.1 | 20.8 | 52.2 | 50.0 | 64.9 | 68.4 |
| cells (%) | | | | | | | | | | | | | | | | |
| KLRG1+ T | 38.08 | 34.74 | 40.23 | 39.74 | 2.04 | 4.58 | 3.72 | 6.17 | 8.14 | 13.01 | 15.68 | 16.65 | 9.36 | 10.67 | 12.25 | 15.16 |
| cells (%) | | | | | | | | | | | | | | | | |

### Example 11: Exemplary Gen 2 Process for Generating PD-1 TIGIT dKO TILs

On day 0, tumor samples are cut into 3 mm fragments and incubated at 37° C. in CM1 with 6000 IU/ml IL-2 in a GREX 6-well plate at 8-12 fragments per well.

On day 7, TransAct at a 1:17.5 dilution is added to the TILs to initiate the stimulation process. Incubate at 37° C. for 2 days.

On day 9, 24 well tissue culture plate is filled with 2 mL of CM1+1000 IU/mL IL-2 in each well for both Mock and PD-1 TAL and kept in the 30° C. incubator at least 30 minutes before starting. 2e6 TILs are resuspend in Electrolytic buffer and electroporated with both the left and right arms of the PD-1 TALEN mRNA at a concentration of 1 μg/1e6 cells. TILs are electroporated using Neon electroporator (ThermoFisher) at 2300 V, 2 ms, 3 pulses and transferred to the pre-warmed 24 well plate with CM1+1000 IU/mL IL-2. 31.) Store the cells at 30° C. overnight. Transfer to 37° C. and incubate for 2 days.

On day 12, the TILs are electroporated with TIGIT TALEN mRNA at a concentration of 1 ug/1e6 cells using the same conditions as for the PD-1 TALEN mRNA, followed by an overnight resting period at 30° C.

On day 13, 17 mL CM2 media, 3000 IU/mL IL-2, 30 ng/ml OKT3 and 25e6 feeders are added to each well in a GREX 6 well plate. 0.1e6 TILs are added to each well. On day 18, 17 mL CM4 media+3000 IU/mL IL-2 is added to each well. TILs are harvested on Day 22.

### Example 12: PD-1 LAG3 dKO TILs Using Concomitant Electroporation

Patient tumors (N=6) from different indications (NSCLC, head & neck, ovarian, and breast) were received, fragmented, and subjected to an 11-day pre-REP process. Following pre-REP cells were stimulated for two days with plate bound OKT3 (300 ng/ml) followed by electroporation of 1e6 cells resuspended in T buffer in 1 mm gap electroporation cuvettes with 4 ug/million cells each of right or left arm TALEN. Following electroporation cells were rested overnight in CM1 media with IL-2 at 30° C. followed by REP. TILs after REP were stimulated overnight with anti-CD3/CD28 beads followed by FACS staining to maximize inhibitory receptor expression.

PD-1 TALEN sequences are described in Table 3. LAG3 TALEN sequences are described in Table 21.

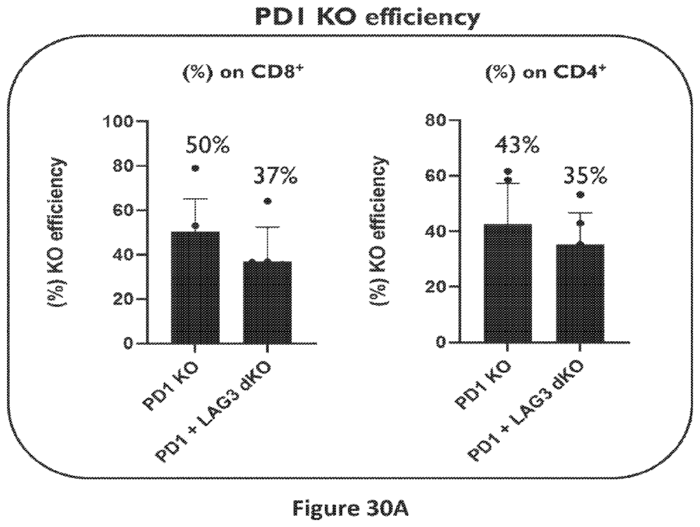

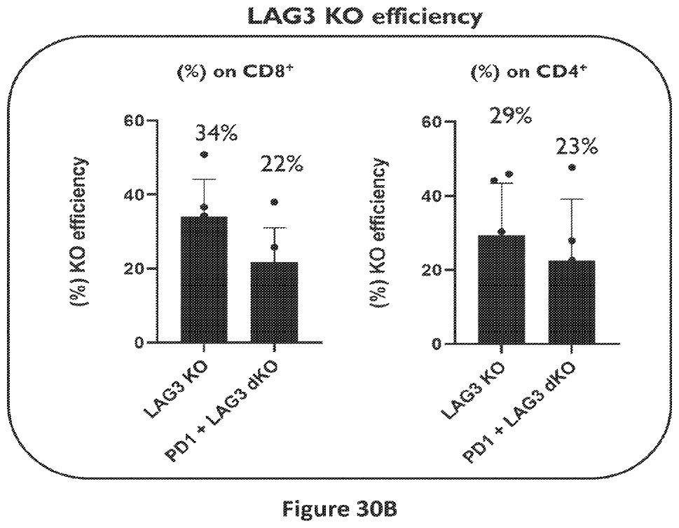

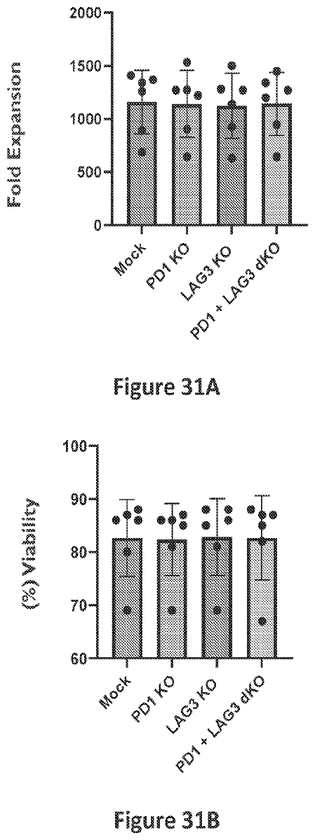

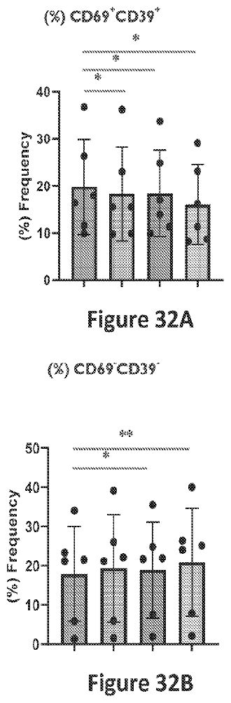

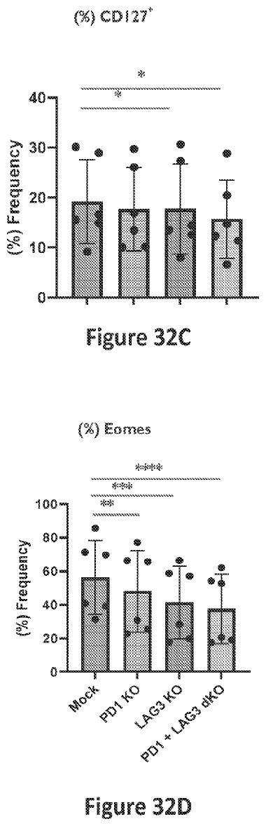

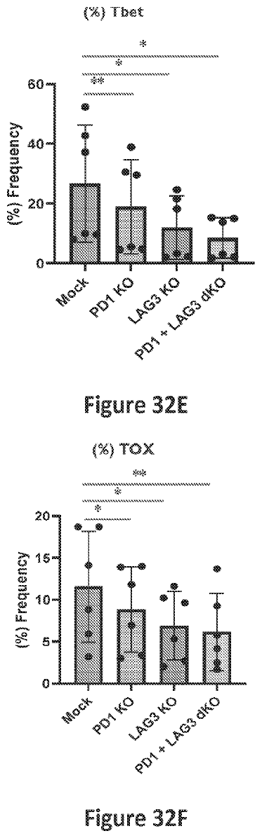

FIGS. 30A and 30B show the single and double KO efficiencies for PD1 and LAG3, respectively. FIGS. 31A and 31B show fold expansion and viability observed for LAG3 single and double KO TILs. FIGS. 32A-32F show decreased CD69, CD39, CD127, Eomes, Tbet and TOX expression in single and double KO TILs. No changes were observed for CD25, CD28, TIM3 and TIGIT expression (data not shown).

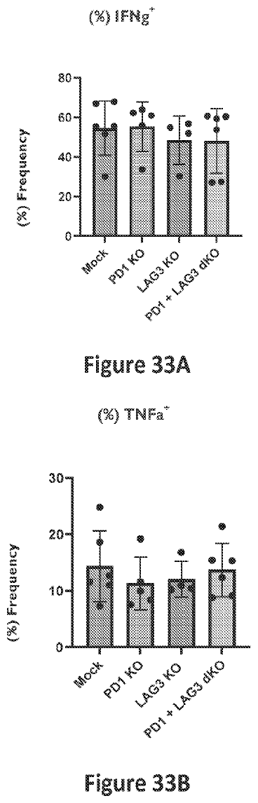

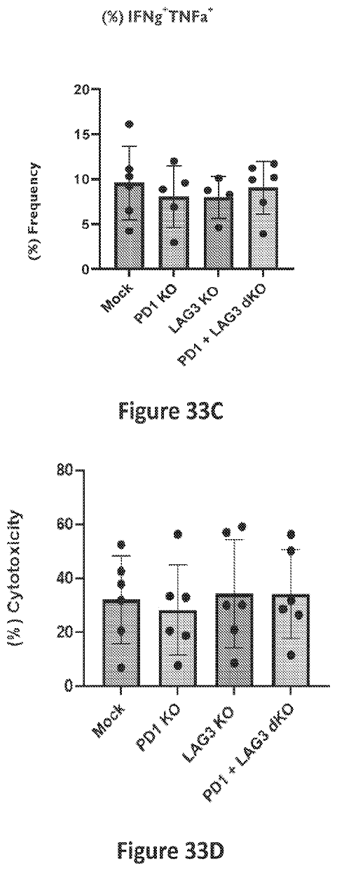

TILs were stimulated overnight with anti-CD3/CD28 beads followed by 5 hr incubation with Brefeldin A. Similar levels of IFNγ and TNFα expression were observed in single and double KO TILs (FIGS. 33A-33C).

TILs were cultured overnight with KILR THP-1 cells for assessment of cytotoxicity. Similar levels of killing activity were observed in single and double KO TILs (FIG. 33D).

### Example 13: PD-1 LAG3 dKO TILs Using Concomitant and Sequential Electroporation

Frozen PreREP cells (N=2) from different indications (Breast and H&N) were thawed. Following pre-REP cells were stimulated for two days with plate bound OKT3 (300 ng/ml) followed by electroporation of 1e6 cells resuspended in T buffer in 1 mm gap electroporation cuvettes with 4 ug/million cells each of right or left arm TALEN. Following electroporation cells were rested overnight in CM1 media with IL-2 at 30° C. followed by REP. Both the concomitant and sequential electroporation process were tested.

PD-1 TALEN sequences are described in Table 3. LAG3 TALEN sequences are described in Table 21.

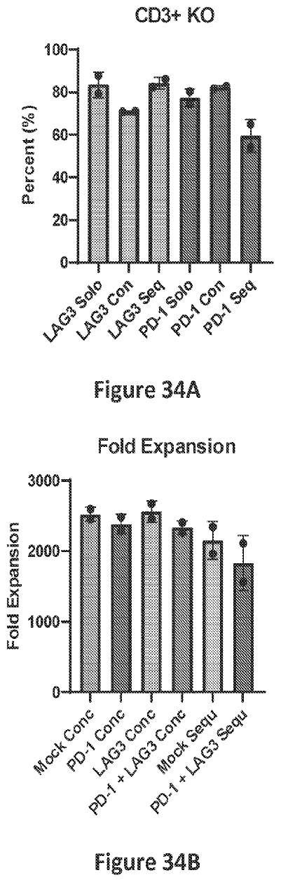

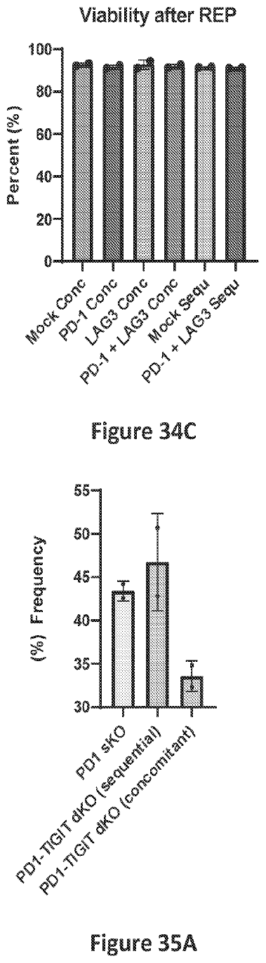

FIGS. 34A-34C show LAG3 and PD1 KO efficiency, fold expansion during REP and viability after REP, respectively.

### Example 14: PD-1 TIGIT dKO and PD-1 LAG3 dKO TILs Using Concomitant and Sequential Electroporation

Pre-REP TIL lines (N=2) from different indications (head & neck and breast) were thawed and activated for 2 days with GMP TransAct 1:17.5 then electroporated in either the concomitant format (2 days after stim) or the sequential format (2 days after stim then again after a 3-day rest).

PD-1 was knocked out during the 1st electroporation step (2 days after stim) and TIGIT or LAG3 was knocked out during the 2nd electroporation step (after the 3 day rest). PD-1 and TIGIT were also knocked out individually to compare single KO efficiency with dKO efficiency. The cells were REP′d then activated with CD3/CD28 beads and stained to determine KO efficiency.

PD-1 TALEN sequences are described in Table 3. TIGIT TALEN sequences are described in Table 4.

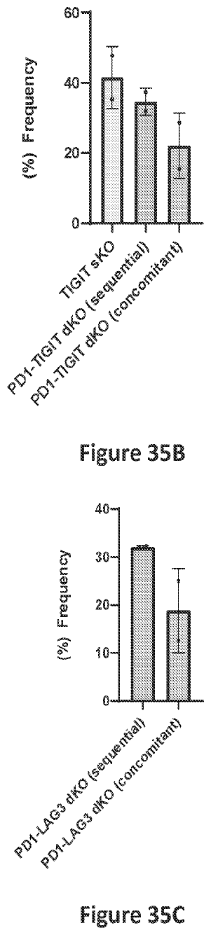

FIGS. 35A-35C show PD-1, TIGIT and LAG3 KO efficiency, respectively.

### Example 15: PD-1 TIGIT On/Off Target mRNA Titration

### Detailed Protocol (for Experiment #10)

PreREP TILs were thawed and counted. TILs were activated for 2 days using 5 mL of GMP TransAct (Miltenyi Biotec cat #170-076-156) in 100 ml of CM1. After activation, TILs were counted and split between the electroporation conditions (below). TILs were washed 1× with PBS and 1× with of CTS™ Xenon™ Genome Editing Buffer (Thermo Fisher, cat #A4998001). TILs were resuspended in CTS™ Xenon™ Genome Editing Buffer and TALEN mRNA (volumes based on the conditions below, 1 mL total volume). Each condition was transferred to a CTS Xenon SingleShot Electroporation Chamber (Thermo Fisher, cat #A50305) and electroporated using the Xenon electroporator with the settings 2300 V, 2 ms pulse width, 3 pulses. TILs were rested in CM2 overnight at 30° C. REPs were set up after the overnight rest using 50e6 iPBMCs, 30 ng/ml MACS® GMP CD3 pure (Miltenyi Biotec, cat #170-076-116), 4e5 TILs, and 100 ml CM2 per condition. After 5 days the scale up was performed by splitting the sample and adding CM4 (100 mL total). TILs were harvested after 4 days and frozen in CS10.

For small scale and full scale runs, PD-1 and TIGIT TALEN mRNA was added at 2 μg/1e6 cells. For Experiments #6-9, thawed REP TILs (in contrast to thawed preREP TILs) were used for electroporation.

PD-1 TALEN sequences are described in Table 3. TIGIT TALEN sequences are described in Table 4.

### Detailed Protocol (for Experiment #10)

### Day 0—Thaw and Activate

1) Frozen preREP vials (D11) TILs were thawed into warm CM1 and counted
2) Cells were transferred into a Grex100M flask in 100 ml of CM1
3) 1 bottle of TransAct (5 mL) was added to the flask
4) Flask was placed in the incubator for 2 days of stimulation at 37° C.

### Day 2—PD-1 Electroporation

1) Cells were harvested from the flask by resuspending the cell suspension and passing through a 70 um cell strainer into a 250 ml conical tube
2) Cell counts were taken
3) Sample was split into the following conditions, each in a 50 ml conical tube:

M1277
Mock with 5M cells
TALEN with 5M cells (2 ug/M cells)
TALEN with 5M cells (1.5 ug/M cells)
TALEN with 5M cells (1 ug/M cells)
TALEN with 10M cells (2 ug/M cells)
TALEN with 10M cells (1 ug/M cells)
TALEN with 15M cells (2 ug/M cells)
TALEN with 15M cells (1 ug/M cells)
TALEN with 15M cells (0.5 ug/M cells)
TALEN with 25M cells (1 ug/M cells)
TALEN with 25M cells (0.5 ug/M cells)
TALEN with 50M cells Mock
TALEN with 50M cells (0.5 ug/M cells)
TALEN with 50M cells (0.25 ug/M cells)
TALEN with 50M cells (0.25 ug/M cells)
TALEN with 100M cells (0.25 ug/M cells)
EP11229
Mock with 5M cells
TALEN with 5M cells (2 ug/M cells)
TALEN with 5M cells (1.5 ug/M cells)
TALEN with 10M cells (2 ug/M cells)
TALEN with 10M cells (1 ug/M cells)
TALEN with 15M cells (2 ug/M cells)
TALEN with 15M cells (1 ug/M cells)
TALEN with 15M cells (0.5 ug/M cells)
TALEN with 25M cells (1 ug/M cells)
TALEN with 25M cells (0.5 ug/M cells)
TALEN with 50M cells (1 ug/M cells)
TALEN with 50M cells (0.5 ug/M cells)
M1280A
Mock with 5M cells
TALEN with 5M cells (2 ug/M cells)
TALEN with 5M cells (1 ug/M cells)
TALEN with 5M cells (0.5 ug/M cells)
TALEN with 10M cells (2 ug/M cells)
TALEN with 10M cells (1 ug/M cells)
TALEN with 10M cells (0.5 ug/M cells)
TALEN with 15M cells (2 ug/M cells)
TALEN with 15M cells (1 ug/M cells)
TALEN with 15M cells (0.5 ug/M cells)
TALEN with 25M cells (1 ug/M cells)
TALEN with 25M cells (0.5 ug/M cells)
TALEN with 50M cells (1 ug/M cells)
TALEN with 50M cells (0.5 ug/M cells)
TALEN with 50M cells (0.25 ug/M cells)
TALEN with 100M cells (0.25 ug/M cells)

4) Cells were washed with PBS and centrifuged 400 g×5 min
5) Cells were washed with 10 mL of CTS™ Xenon™ Genome Editing Buffer (Thermo Fisher, cat #A4998001) and centrifuged 400 g×5 min
6) Cells were resuspended in CTS™ Xenon™ Genome Editing Buffer and mRNA (total of 1 mL) and were electroporated using the Xenon electroporator with the settings 2300 V, 2 ms pulse width, 3 pulses 7) Cells were rested in CM2 at 30° C. overnight

### Day 3—REP

1) Cells were collected and counted
2) 4e5 TIL were plated in a GREX 6 well plate, 1 well per condition in 100 ml of CM2
3) 50e6 iPBMCs+30 ng/ml of OKT3 were added to each well
4) Cells were incubated at 37° C.

### Day 8—Scale Up

1) Each well was volume reduced by ~80 mL
2) Each well was resuspended and transferred to a 50 ml conical tube
3) ½ of the sample was replated in the Grex 6 well plate
4) CM4 was added to each well to 100 ml total volume

### Day 12—Harvest

1) Each well was volume reduced by ~80 mL
2) Each well was resuspended and transferred to a 50 ml conical tube
1) Cell counts were taken
2) Cells were frozen at ~10e6 cells/ml in 1 ml of CS10

### DNA Sequencing

DNA was isolated from harvested TILs using the QIAamp DNA Blood Mini Kit (Qiagen, Cat #: 51106), followed by the library prep for Next Gen Sequencing using the CleanPlex Custom Panel Kit (Paragon Genomics, Cat #: 937001). All DNA samples were sequenced on Illumina NextSeq2000 using either NextSeq 1000/2000 P1 Reagents-300 Cycles (Illumina, Cat #20050264) or NextSeq 1000/2000 P2 Reagents-300 Cycles (Illumina, Cat #20046813), using paired end 2×150 bp reads.

### Sequencing Analysis

Targeted DNA sequencing analysis was performed using the R package ampliCan1 (v1.22.1). Local alignment of the fastq reads to expected amplicon sequences was done using the amplicanAlignfunction with default parameters. Alignment events observed in overlapping primer or primer-dimer infected reads were filtered out. Insertions and deletions (indels) were then quantified per sample. Further analysis was performed via a custom python script to further summarize and normalize the data. TALEN associated edits at each targeted region of interest were calculated by the subtraction of frequency of indels in non-edited control samples from the signals observed in TALEN treated samples.

Observed on-target and candidate off-target edits in individual samples from various experiments were analyzed further relative to their corresponding mRNA concentration used for electroporation. Individual values converted to ug mRNA per mL solution were plotted and nonlinear regressions were calculated using hyperbolic interpolation using GraphPad Prism. In each generated curve, the point of inflection demonstrates where an increase in mRNA concentration no longer substantially increases the edit rate. The optimal mRNA concentration maximizes the edit for the on-target sites, and minimizes signals observed for the candidate off-target sites.

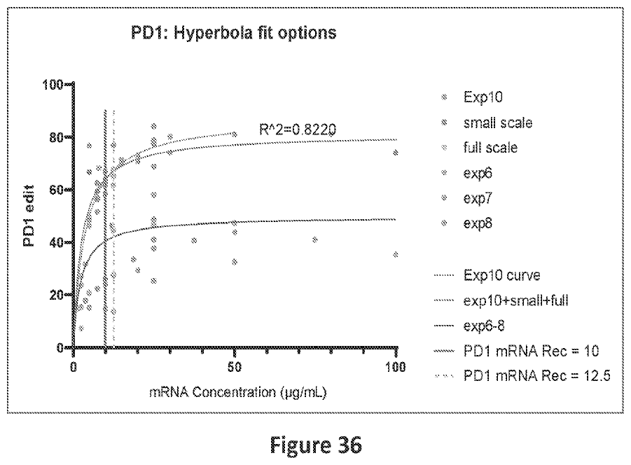

FIG. 36 shows PD-1 on-target hyperbola fit options. Observed PD-1 edit rates are indicated with dots for samples' corresponding mRNA concentration (ug/mL) used for electroporation. Non-linear regressions, using hyperbola interpolations, were derived for groups of experiments and are identified with the curved lines. Example recommendations of mRNA concentrations (ug/mL) are identified with vertical lines at 10 μg/ml (green) and 12.5 μg/mL (orange dotted line). This discovery identifies that the selection of mRNA concentration, irrespective of the number of cells, can be used to optimize KO efficiency for on-target editing.

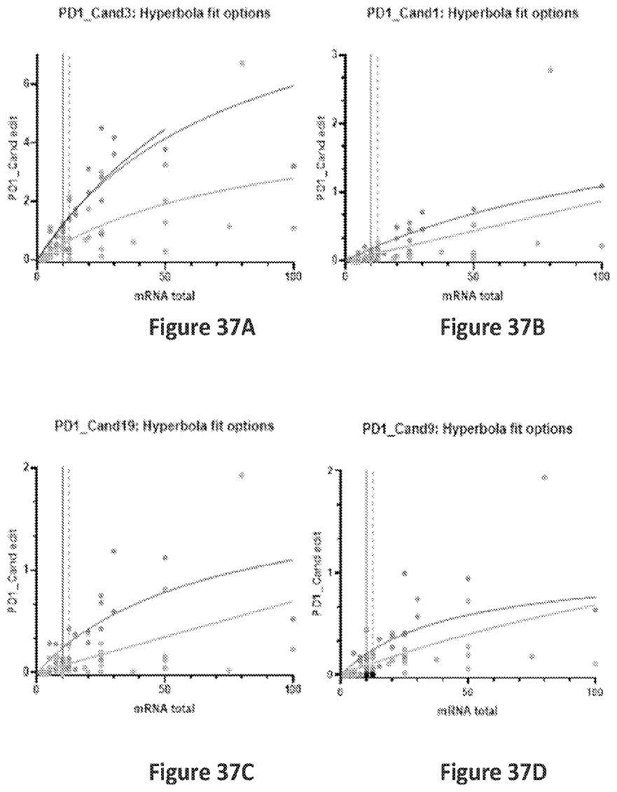

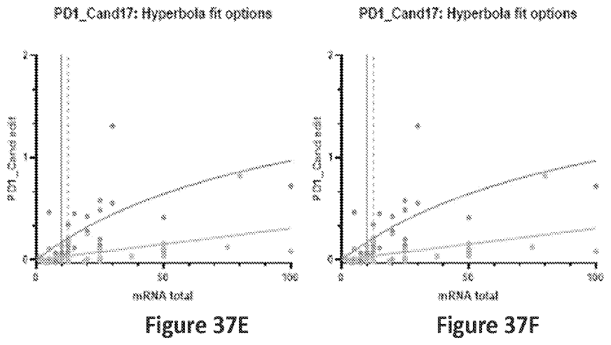

FIGS. 37A-37F show PD-1 off-target signals for Candidates 3, 1, 19, 9, 17, and 4, respectively. Observed edit rates for select PD-1 candidate off-targets are indicated with dots for samples' corresponding mRNA concentration (ug/mL) used for electroporation. Non-linear regressions, using hyperbola interpolations, were derived for groups of experiments and are identified with the curved lines. Example recommendations of mRNA concentrations (ug/mL) are identified with vertical lines at 10 μg/ml (green) and 12.5 μg/mL (orange dotted line). The discovery identifies that selection of mRNA concentration, irrespective of the number of cells, can be used to minimize off-target editing.

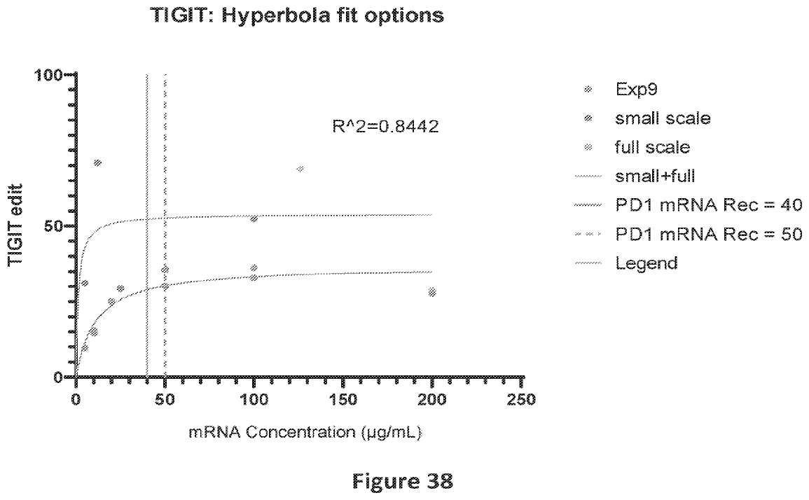

FIG. 38 shows TIGIT on-target hyperbola fit options. Observed edit rates for TIGIT are indicated with dots for samples' corresponding mRNA concentration (ug/mL) used for electroporation. Non-linear regressions, using hyperbola interpolations, were derived for groups of experiments and are identified with the curved lines. Example recommendations of mRNA concentrations (ug/mL) are identified with vertical lines at 40 μg/mL (green) and 50 μg/mL (blue dotted line). This discovery identifies that the selection of mRNA concentration, irrespective of the number of cells, can be used to optimize KO efficiency for on-target editing.

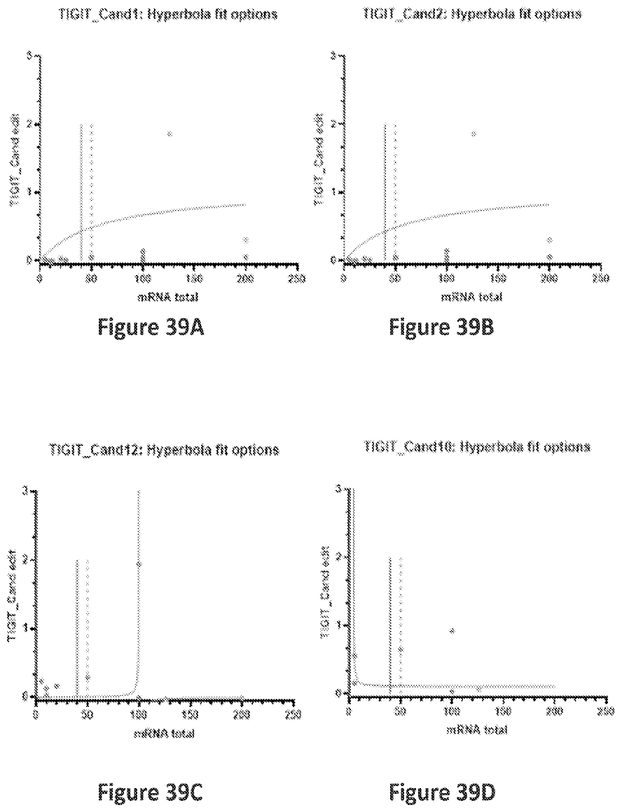

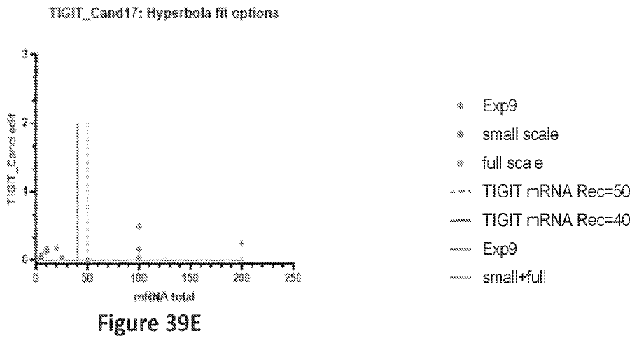

FIGS. 39A-39E show TIGIT off-target signals for Candidates 1, 2, 10, 12, and 17, respectively. Observed edit rates for select TIGIT candidate off-targets are indicated with dots for samples' corresponding mRNA concentration (ug/mL) used for electroporation. Non-linear regressions, using hyperbola interpolations, were derived for groups of experiments and are identified with the curved lines. Example recommendations of mRNA concentrations (ug/mL) are identified with vertical lines at 40 μg/ml (green) and 50 μg/mL (blue dotted line). The discovery identifies that selection of mRNA concentration, irrespective of the number of cells, can be used to minimize off-target editing.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method for preparing expanded tumor infiltrating lymphocytes (TILs) obtained from a tumor resected from a patient having reduced expression of PD-1 and TIGIT, comprising:
(a) culturing a first population of TILs from the tumor from the patient in a first cell culture medium comprising IL-2 for about 5-7 days to produce a second population of TILs;
(b) activating the second population of TILs for about 2-4 days, to produce a third population of TILs;
(c) introducing a first TALE nuclease (TALEN) system targeting a first gene selected from the group consisting of a gene encoding PD-1 and a gene encoding TIGIT into at least a portion of the third population of TILs, to produce a fourth population of TILs;
(d) resting the fourth population of TILs in the first cell culture medium comprising IL-2 for about 2 to 3 days;
(e) introducing a second TALEN system targeting a second gene selected from the group consisting of the gene encoding PD-1 and the gene encoding TIGIT into at least a portion of the fourth population of TILs to produce a fifth population of TILs, wherein the first gene and the second gene are different; and
(f) culturing the fifth population of TILs in a second cell culture medium comprising antigen presenting cells (APCs), OKT-3, and IL-2 for about 7-11 days, to produce sixth population of TILs having reduced expression of the first gene and the second gene.

2. The method of claim 1, wherein the step of activating the second population of TILs is performed for about 2 days, or about 3 days or about 4 days; or
wherein the step of culturing the first population of TILs is performed for about 5 days or about 6 days or about 7 days; or
wherein the step of culturing the fifth population of TILs is performed for about 7 days, about 8 days, about 9 days, about 10 days or about 11 days.

3.-12. (canceled)

13. The method of claim 1, wherein all steps are completed within a period of about 21 days or wherein all steps are completed within a period of about 19-22 days; or wherein all steps are completed within a period of about 19-21 days; wherein all steps are completed within a period of about 20-22 days.

14.-16. (canceled)

17. The method of claim 1, further comprising an overnight resting step after introducing the first and/or the second TALE nuclease system.

18.-20. (canceled)

21. The method of claim 1, wherein the step of activating the second population of TILs is performed using an anti-CD3 agonist and an anti-CD28 agonist.

22. (canceled)

23. The method of claim 21, wherein the step of activating the second population of TILs is performed using TransAct at 1:17.5 dilution.

24.-25. (canceled)

26. The method of claim 24 or 251, wherein the target sequence of the PD-1 targeting TALEN system comprise the nucleic acid sequence of SEQ ID NO. 18 and the target sequence of the TIGIT targeting TALEN system comprise the nucleic acid sequence of SEQ ID NO. 23 or 28.

27. The method of claim 1, wherein the first TALEN system comprises a first pair of half-TALEs targeting the first gene, wherein the second TALEN system comprises a second pair of half-TALEs targeting the second gene, and wherein the introducing of the first TALEN system comprises a first electroporation of the third population of TILs with a first pair of mRNAs encoding the first pair of half-TALEs and/or the introducing of the second TALEN system comprises a second electroporation of the fifth population of TILs with a second pair of mRNAs encoding the second pair of half-TALEs.

28. The method of claim 27, wherein the first pair of half-TALEs comprise the amino acid sequences of SEQ ID NO: 15 and 17.

29. The method of claim 27, wherein the second pair of half-TALEs comprise the amino acid sequences of SEQ ID NO: 20 and 22.

30. The method of claim 27, wherein the second pair of half-TALEs comprise the amino acid sequences of SEQ ID NO: 25 and 27.

31. The method of claim 27, wherein in the first electroporation the first pair of mRNAs is introduced at about 1-2 μg mRNA/million cells of the third population of TILs and/or in the second electroporation the second pair of mRNAs is introduced at about 1-2 μg mRNA/million cells of the fifth population of TILs.

32. The method of claim 1, wherein step (c) is preceded by washing the third population of TILs in a cytoporation buffer.

33-81. (canceled)

82. A gene-edited population of tumor infiltrating lymphocytes (TILs) comprising an expanded population of TILs having reduced expression of the first gene and the second gene produced by the method of the method of claim 1.

83. The gene-edited population of TILs of claim 82, wherein about 64% of the expanded population of TILs comprises knockout of both PD-1 and TIGIT.

84.-85. (canceled)

86. A pharmaceutical composition comprising the gene edited population of TILs of claim 82 and a pharmaceutically acceptable carrier.

87. A method for treating a cancer patient, the method comprising administering a therapeutically effective dose of the gene edited population of TILs of claim 82 into the cancer patient.

88. (canceled)

89. A method for treating a cancer patient, comprising:
(a) obtaining a first population of TILs from a tumor sample resected from the cancer patient by processing a tumor sample obtained from the cancer patient into multiple tumor fragments, or from a tumor sample obtained from the cancer patient by surgical resection, needle biopsy, core biopsy, small biopsy, or other means;
(b) culturing the first population of TILs in a first cell culture medium comprising IL-2 for about 5-7 days to produce a second population of TILs;
(c) activating the second population of TILs for about 2-4 days, to produce a third population of TILs;
(d) introducing a first TALE nuclease (TALEN) system targeting a first gene selected from the group consisting of a gene encoding PD-1 and a gene encoding TIGIT into at least a portion of the third population of TILs, to produce a fourth population of TILs;
(e) resting the fourth population of TILs in the first cell culture medium comprising IL-2 for about 2 to 3 days;
(f) introducing a second TALEN system targeting a second gene selected from the group consisting of the gene encoding PD-1 and the gene encoding TIGIT into at least a portion of the fourth population of TILs to produce a fifth population of TILs, wherein the first gene and the second gene are different;
(g) culturing the fifth population of TILs in a second cell culture medium comprising antigen presenting cells (APCs), OKT-3, and IL-2 for about 7-11 days, to produce sixth population of TILs having reduced expression of the first gene and the second gene; and
(h) administering a therapeutically effective dosage of the sixth population of TILs to the cancer patient.

90.-190. (canceled)
